(19) United States
(12) Patent Application Publication
(10) Pub. No.: US 2022/0315907 A1
(43) Pub. Date: Oct. 6, 2022

(54) SPLIT CRISPR NUCLEASE TETHERING SYSTEM

(71) Applicant: Inscripta, Inc., Boulder, CA (US)

(72) Inventor: Andrew Garst, Boulder, CO (US)

(21) Appl. No.: 17/639,022

(22) PCT Filed: Oct. 1, 2020
(86) PCT No.: PCT/US2020/053873

§ 371 Date: Feb. 28, 2022

Related U.S. Application Data
(60) Provisional application No. 62/913,715, filed on Oct. 10, 2019.

(51) Int. Cl. C12N 9/22; C12N 15/11; C12N 15/10

(52) U.S. Cl. CPC C12N 9/22; C12N 15/11; C12N 15/102; C12N 2310/20

(57) ABSTRACT

The present disclosure provides compositions and methods to increase the percentage of edited cells in a cell population when employing nucleic-acid guided editing, as well as automated multi-module instruments for performing these methods.

### RELATED CASES

The present application is a US 371 National Phase filing of International PCT/US20/53873, filed 1 Oct. 2020, which claims priority to U.S. Ser. No. 62/913,715, filed 10 Oct. 2019, entitled “Split Nuclease Tethering System.”

### FIELD OF THE INVENTION

The present disclosure relates to methods and compositions to increase the percentage of edited cells in a cell population when employing nucleic-acid guided editing, as well as automated multi-module instruments for performing these methods and using these compositions.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

The ability to make precise, targeted changes to the genome of living cells has been a long-standing goal in biomedical research and development. Recently, various nucleases have been identified that allow for manipulation of gene sequences, and hence gene function. The nucleases include nucleic acid-guided nucleases, which enable researchers to generate permanent edits in live cells. Of course, it is desirable to attain the highest editing rates possible in a cell population; however, in many instances the percentage of edited cells resulting from nucleic acid-guided nuclease editing can be in the single digits.

There is thus a need in the art of nucleic acid-guided nuclease editing for improved methods, compositions, modules and instruments for increasing the efficiency of editing. The present disclosure addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to methods, compositions, modules and automated multi-module cell processing instruments that increase the efficiency nucleic acid-guided editing in a cell population using a split nuclease tethering system. In this system, an RNA-guided nuclease is “split” at a point where there is no spontaneous association of the N-terminal and C-terminal portions in the absence of the ligand and a transcription factor (TF) binding site on the editing vector but where there is association of the N-terminal and C-terminal portions of the nuclease and reconstitution of nuclease activity in the presence of the ligand and upon binding of the dimerized transcription factor at the transcription factor binding site. The present methods and compositions increase the efficiency of nucleic acid-guided editing by both allowing for control over the timing of the editing process in the cells and by physically tethering the target site for the edit to the editing vector thereby increasing the local concentration of donor DNA for templating homologous repair (HR). Despite precedent for split nuclease enzyme functionality, other embodiments have focused on the use of small molecule inducers but fail to provide a logic that that spatially links the HR donor repair substrate to the site of the dsDNA break in order to promote high efficiency precision editing. The systems described herein thus provide a general means to control both the temporal and spatial induction of homologous repair for such genome editing workflows.

Thus, some embodiments provide a nucleic acid-guided nuclease editing system comprising: an N-terminal nuclease domain/first transcription factor fusion construct present in a vector backbone; a C-terminal nuclease domain/second transcription factor fusion construct present on a vector backbone, wherein the first transcription factor and the second transcription factor associate with one another in the presence of a ligand; an editing cassette comprising a gRNA transcription sequence and a donor DNA transcription sequence present in a vector backbone; and a binding site for the associated first and second transcription factors present in a vector backbone. In some aspects, one vector back bone further comprises a coding sequence for the ligand, and in some aspects, the coding sequence for the ligand is under the control of an inducible promoter. In some aspects, the N-terminal nuclease domain/first transcription factor fusion construct and the C-terminal nuclease domain/second transcription factor fusion construct are present on a first vector and the editing cassette and the binding site for the associated first and second transcription factors are present on a second vector and in some aspects the N-terminal nuclease domain/first transcription factor fusion construct and the C-terminal nuclease domain/second transcription factor fusion construct are under the control of a single promoter. In other aspects, the N-terminal nuclease domain/first transcription factor fusion construct and the C-terminal nuclease domain/second transcription factor fusion construct are under the control of separate promoters.

In some aspects of this embodiment, the first and second transcription factors are a same transcription factor that dimerize in the presence of the ligand, and in some aspects, the transcription factors are selected from AraR, XlrR, AP-1 (activator protein 1), C/EBP (CCAAT-enhancer binding protein, ATF/CREB activating transcription factor/cAMP response binding element, c-Myc, or NF-1 nuclear factor 1.

In some aspects, the N-terminal nuclease domain and the C-terminal nuclease domains are derived from MAD7 nuclease, and in some aspects, the N-terminal and C-terminal nuclease domains are derived from another MAD nuclease. In some aspects, the MAD7 nuclease is split in a position as enumerated in Table 2.

Also is provided a cell comprising the N-terminal nuclease domain/first transcription factor fusion construct present in a vector backbone; the C-terminal nuclease domain/second transcription factor fusion construct present on a vector backbone, wherein the first transcription factor and the second transcription factor associate with one another in the presence of a ligand; the editing cassette comprising a gRNA transcription sequence and a donor DNA transcription sequence present in a vector backbone; and the binding site for the associated first and second transcription factors present in a vector backbone, and in some aspects, the cell comprising the nucleic acid-guided editing system has been edited.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A is a simple process diagram for nucleic acid-guided nuclease editing using a first vector comprising an N-terminal nuclease peptide and a C-terminal nuclease peptide where each peptide is attached via a linker to a transcription factor (e.g., a “split nuclease tethering system”) and a second vector comprising an editing cassette and a binding site for the transcription factor (“editing vector”). FIG. 1B is a simplified schematic of the components of a split nuclease tethering system used to perform nucleic acid-guided nuclease editing.

FIGS. 2A-2C depict three different views of an exemplary automated multi-module cell processing instrument for performing nucleic acid-guided nuclease editing employing a split nuclease tethering system.

FIG. 3A depicts one embodiment of a rotating growth vial for use with the cell growth module described herein and in relation to FIGS. 3B-3D. FIG. 3B illustrates a perspective view of one embodiment of a rotating growth vial in a cell growth module housing. FIG. 3C depicts a cut-away view of the cell growth module from FIG. 3B. FIG. 3D illustrates the cell growth module of FIG. 3B coupled to LED, detector, and temperature regulating components.

FIG. 4A depicts retentate (top) and permeate (bottom) members for use in a tangential flow filtration module (e.g., cell growth and/or concentration module), as well as the retentate and permeate members assembled into a tangential flow assembly (bottom). FIG. 4B depicts two side perspective views of a reservoir assembly of a tangential flow filtration module. FIGS. 4C-4D depict an exemplary top, with fluidic and pneumatic ports and gasket suitable for the reservoir assemblies shown in FIG. 4B.

FIG. 5A depicts an exemplary combination reagent cartridge and electroporation device (e.g., transformation module) that may be used in a multi-module cell processing instrument. FIG. 5B is a top perspective view of one embodiment of an exemplary flow-through electroporation device that may be part of a reagent cartridge. FIG. 5C depicts a bottom perspective view of one embodiment of an exemplary flow-through electroporation device that may be part of a reagent cartridge. FIGS. 5D-5F depict a top perspective view, a top view of a cross section, and a side perspective view of a cross section of an FTEP device useful in a multi-module automated cell processing instrument such as that shown in FIGS. 2A-2C.

FIG. 6A depicts a simplified graphic of a workflow for singulating, editing and normalizing cells in a solid wall device. 6B depicts a simplified graphic of a workflow variation for substantially singulating, editing and normalizing cells in a solid wall device. FIGS. 6C-6E depict an embodiment of a solid wall isolation incubation and normalization (SWIIN) module. FIG. 6F depicts the embodiment of the SWIIN module in FIGS. 6C-6E further comprising a heater and a heated cover.

FIG. 7 is a simplified process diagram of an embodiment of an exemplary automated multi-module cell processing instrument in which the split nuclease tethering system described herein may be used.

FIG. 8 is a simplified process diagram of another embodiment of an exemplary automated multi-module cell processing instruments in the split nuclease tethering system described herein may be used.

FIG. 9A shows the split nuclease tethering system bound to a transcription factor binding site on an editing vector and to a target site in a genome to be edited. FIG. 9B depicts exemplary engine and editing vectors for split nuclease editing in E. coli. FIG. 9C depicts the expected readouts for results in a gel shift application for seven different experimental variations.

It should be understood that the drawings are not necessarily to scale, and that like reference numbers refer to like features.

### DETAILED DESCRIPTION

All of the functionalities described in connection with one embodiment of the methods, devices or instruments described herein are intended to be applicable to the additional embodiments of the methods, devices and instruments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York, 1989; and Chang, et al., Guide to Electroporation and Electrofusion, Academic Press, California (1992), all of which are herein incorporated in their entirety by reference for all purposes. Nucleic acid-guided nuclease techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

The term “complementary” as used herein refers to Watson-Crick base pairing between nucleotides and specifically refers to nucleotides hydrogen-bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TTAGCTGG-3′.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and—for some components—translated in an appropriate host cell.

As used herein the term “donor DNA” or “donor nucleic acid” refers to nucleic acid that is designed to introduce a DNA sequence modification (insertion, deletion, substitution) into a locus (e.g., a target genomic DNA sequence or cellular target sequence) by homologous recombination using nucleic acid-guided nucleases. For homology-directed repair, the donor DNA must have sufficient homology to the regions flanking the “cut site” or site to be edited in the genomic target sequence. The length of the homology arm(s) will depend on, e.g., the type and size of the modification being made. In many instances and preferably, the donor DNA will have two regions of sequence homology (e.g., two homology arms) to the genomic target locus. Preferably, an “insert” region or “DNA sequence modification” region—the nucleic acid modification that one desires to be introduced into a genome target locus in a cell—will be located between two regions of homology. The DNA sequence modification may change one or more bases of the target genomic DNA sequence at one specific site or multiple specific sites. A change may include changing 1, 2, 3, 4, 5, 10, 15, 20, 25, 30, 35, 40, 50, 75, 100, 150, 200, 300, 400, or 500 or more base pairs of the genomic target sequence. A deletion or insertion may be a deletion or insertion of 1, 2, 3, 4, 5, 10, 15, 20, 25, 30, 40, 50, 75, 100, 150, 200, 300, 400, or 500 or more base pairs of the genomic target sequence.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” or “homology arm” refers to a region on the donor DNA with a certain degree of homology with the target genomic DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

As used herein, the terms “protein” and “polypeptide” are used interchangeably. Proteins may or may not be made up entirely of amino acids.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA transcribed by any class of any RNA polymerase I, II or III. Promoters may be constitutive or inducible.

As used herein the term “selectable marker” refers to a gene introduced into a cell, which confers a trait suitable for artificial selection. General use selectable markers are well-known to those of ordinary skill in the art and include ampilcillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 or other selectable markers may be employed.

The term “specifically binds” as used herein includes an interaction between two molecules, e.g., an engineered peptide antigen and a binding target, with a binding affinity represented by a dissociation constant of about 10⁻⁷M, about 10⁻⁸M, about 10⁻⁹ M, about 10⁻¹⁰ M, about 10⁻¹¹M, about 10⁻¹²M, about 10⁻¹³M, about 10⁻¹⁴M or about 10⁻¹⁵M.

The term “split nuclease tethering system” refers to a system for nucleic acid-guided nuclease editing comprising a nucleic acid-guided nuclease that is separated or “split” into N-terminal and C-terminal peptides or portions, where each of the N-terminal and C-terminal peptides are linked, via a peptide linker, to a transcription factor that dimerizes in the presence of an appropriate ligand. Thus, in the presence of the appropriate ligand, the transcription factors dimerize thereby bringing the N-terminal and C-terminal peptides of the nucleic acid-guided nuclease into spatial proximity so as to function as a nucleic acid-guided nuclease. In a split nuclease tethering system, the editing vector comprises 1) a CREATE cassette (e.g., see U.S. Ser. Nos. 9,982,278; 10,266,849; and 10,240,167, and 15/948,785; 16/056,310; 16,275,439; and Ser. No. 16/275,465) where the CREATE cassette comprises a sequence for a gRNA and a donor DNA, and 2) a binding site for the dimerized transcription factor.

The terms “target genomic DNA sequence”, “cellular target sequence”, “target sequence”, or “genomic target locus” refer to any locus in vitro or in vivo, or in a nucleic acid (e.g., genome or episome) of a cell or population of cells, in which a change of at least one nucleotide is desired using a nucleic acid-guided nuclease editing system. The target sequence can be a genomic locus or extrachromosomal locus.

The term “variant” may refer to a polypeptide or polynucleotide that differs from a reference polypeptide or polynucleotide but retains essential properties. A typical variant of a polypeptide differs in amino acid sequence from another reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A variant of a polypeptide may be a conservatively modified variant. A substituted or inserted amino acid residue may or may not be one encoded by the genetic code (e.g., a non-natural amino acid). A variant of a polypeptide may be naturally occurring, such as an allelic variant, or it may be a variant that is not known to occur naturally.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, synthetic chromosomes, and the like. In some embodiments, two vectors—an engine vector, comprising the coding sequences (N-terminal and C-terminal) for a nuclease linked to transcription factors, and an editing vector, comprising the gRNA sequence, the donor DNA sequence, and a binding site for the dimerized transcription factor—are used. In alternative embodiments, all editing components, including the N-terminal and C-terminal portions of the nuclease each linked to a transcription factor, gRNA sequence, donor DNA sequence, and binding site for the dimerized transcription factor are all on the same vector (e.g., a combined editing/engine vector). In yet another alternative embodiment, a three vector system may be used where two engine vectors—each with the coding sequence for one of the nuclease/transcription factor fusion peptides—and one editing vector (including the gRNA sequence, donor DNA sequence, and binding site for the dimerized transcription factor) is employed.

### Nuclease-Directed Genome Editing Generally

The compositions and methods described herein are employed to perform nuclease-directed genome editing to introduce desired edits to a population of cells. In some embodiments, recursive cell editing is performed where edits are introduced in successive rounds of editing. A nucleic acid-guided nuclease complexed with an appropriate synthetic guide nucleic acid in a cell can cut the genome of the cell at a desired location. The guide nucleic acid helps the nucleic acid-guided nuclease recognize and cut the DNA at a specific target sequence (either a cellular target sequence or a curing target sequence). By manipulating the nucleotide sequence of the guide nucleic acid, the nucleic acid-guided nuclease may be programmed to target any DNA sequence for cleavage as long as an appropriate protospacer adjacent motif (PAM) is nearby. In certain aspects, the nucleic acid-guided nuclease editing system may use two separate guide nucleic acid molecules that combine to function as a guide nucleic acid, e.g., a CRISPR RNA (crRNA) and trans-activating CRISPR RNA (tracrRNA). In other aspects and preferably, the guide nucleic acid is a single guide nucleic acid construct that includes both 1) a guide sequence capable of hybridizing to a genomic target locus, and 2) a scaffold sequence capable of interacting or complexing with a nucleic acid-guided nuclease.

In general, a guide nucleic acid (e.g., gRNA) complexes with a compatible nucleic acid-guided nuclease and can then hybridize with a target sequence, thereby directing the nuclease to the target sequence. A guide nucleic acid can be DNA or RNA; alternatively, a guide nucleic acid may comprise both DNA and RNA. In some embodiments, a guide nucleic acid may comprise modified or non-naturally occurring nucleotides. In cases where the guide nucleic acid comprises RNA, the gRNA may be encoded by a DNA sequence on a polynucleotide molecule such as a plasmid, linear construct, or the coding sequence may and preferably does reside within an editing cassette. For additional information regarding editing cassettes, see U.S. Ser. Nos. 9,982,278; 10,266,849; and 10,240,167, and U.S. Ser. Nos. 15/948,785; 16/056,310; 16,275,439; and Ser. No. 16/275,465, all of which are incorporated by reference herein.

A guide nucleic acid comprises a guide sequence, where the guide sequence is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In general, to generate an edit in the target sequence, the gRNA/nuclease complex binds to a target sequence as determined by the guide RNA, and the nuclease recognizes a protospacer adjacent motif (PAM) sequence adjacent to the target sequence. The target sequence can be any polynucleotide endogenous or exogenous to the cell, or in vitro. For example, the target sequence can be a polynucleotide residing in the nucleus of the cell. A target sequence can be a sequence encoding a gene product (e.g., a protein) or a non-coding sequence (e.g., a regulatory polynucleotide, an intron, a PAM, a control sequence, or “junk” DNA).

The guide nucleic acid may be and preferably is part of an editing cassette that encodes the donor nucleic acid that targets a cellular target sequence. Alternatively, the guide nucleic acid may not be part of the editing cassette and instead may be encoded on the editing vector backbone. For example, a sequence coding for a guide nucleic acid can be assembled or inserted into a vector backbone first, followed by insertion of the donor nucleic acid in, e.g., an editing cassette. In other cases, the donor nucleic acid in, e.g., an editing cassette can be inserted or assembled into a vector backbone first, followed by insertion of the sequence coding for the guide nucleic acid. Preferably, the sequence encoding the guide nucleic acid and the donor nucleic acid are located together in a rationally-designed editing cassette and are simultaneously inserted or assembled via gap repair into a linear plasmid or vector backbone to create an editing vector.

The target sequence is associated with a proto-spacer mutation (PAM), which is a short nucleotide sequence recognized by the gRNA/nuclease complex. The precise preferred PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering of the PAM-interacting domain of a nucleic acid-guided nuclease may allow for alteration of PAM specificity, improve target site recognition fidelity, decrease target site recognition fidelity, or increase the versatility of a nucleic acid-guided nuclease.

In most embodiments, the genome editing of a cellular target sequence both introduces a desired DNA change to a cellular target sequence, e.g., the genomic DNA of a cell, and removes, mutates, or renders inactive a proto-spacer mutation (PAM) region in the cellular target sequence (e.g., renders the target site immune to further nuclease binding). Rendering the PAM at the cellular target sequence inactive precludes additional editing of the cell genome at that cellular target sequence, e.g., upon subsequent exposure to a nucleic acid-guided nuclease complexed with a synthetic guide nucleic acid in later rounds of editing. Thus, cells having the desired cellular target sequence edit and an altered PAM can be selected for by using a nucleic acid-guided nuclease complexed with a synthetic guide nucleic acid complementary to the cellular target sequence. Cells that did not undergo the first editing event will be cut rendering a double-stranded DNA break, and thus will not continue to be viable. The cells containing the desired cellular target sequence edit and PAM alteration will not be cut, as these edited cells no longer contain the necessary PAM site and will continue to grow and propagate.

As for the nuclease component of the nucleic acid-guided nuclease editing system, a polynucleotide sequence encoding the nucleic acid-guided nuclease can be codon optimized for expression in particular cell types, such as bacterial, yeast, and mammalian cells. The choice of nucleic acid-guided nuclease to be employed depends on many factors, such as what type of edit is to be made in the target sequence and whether an appropriate PAM is located close to the desired target sequence. In the present compositions and methods, the nuclease is “split” into N-terminal and C-terminal portions, each of which is tethered or fused to a transcription factor, where the transcription factors dimerize in the presence of a ligand. The nuclease is “split” at a point where there is no spontaneous association of the N-terminal and C-terminal portions in the absence of the ligand and a transcription factor binding site on the editing vector but where there is association of the N-terminal and C-terminal portions and reconstitution of nuclease activity in the presence of the ligand and upon binding of the dimerized transcription factor at the transcription factor binding site. Nucleases of use in the methods described herein include but are not limited to Cas 9, Cas 12/CpfI, MAD2, or MAD7 or other MADzymes.

As with the guide nucleic acid, the N-terminal and C-terminal portions of the nuclease are encoded by a DNA sequence on a vector and optionally is under the control of an inducible promoter. In some embodiments, the promoter may be separate from but the same as the promoter controlling transcription of the guide nucleic acid; that is, a separate promoter drives the transcription of the portions of the nuclease and transcription factor fusions and guide nucleic acid sequences but the two promoters may be the same type of promoter. Alternatively, the promoter controlling transcription of the portions of the nuclease and transcription factor fusions may be different from the promoter controlling transcription of the guide nucleic acid; that is, e.g., the nuclease fusions may be under the control of, e.g., the pBAD promoter, and the guide nucleic acid may be under the control of the, e.g., pL promoter.

Another component of the nucleic acid-guided nuclease system is the donor nucleic acid comprising homology to the cellular target sequence. The donor nucleic acid is on the same vector and even in the same editing cassette as the guide nucleic acid and preferably is (but not necessarily is) under the control of the same promoter as the editing gRNA (that is, a single promoter driving the transcription of both the editing gRNA and the donor nucleic acid). The donor nucleic acid is designed to serve as a template for homologous recombination with a cellular target sequence nicked or cleaved by the nucleic acid-guided nuclease as a part of the gRNA/nuclease complex. A donor nucleic acid polynucleotide may be of any suitable length, such as about or more than about 20, 25, 50, 75, 100, 150, 200, 500, or 1000 nucleotides in length, and up to 2, 3, 4, 5, 6, 7, 8, 9, 10, 11, 12, 13 and up to 20 kb in length if combined with a dual gRNA architecture as described in U.S. Ser. No. 16/275,465, filed 14 Feb. 2019. In certain preferred aspects, the donor nucleic acid can be provided as an oligonucleotide of between 20-300 nucleotides, more preferably between 50-250 nucleotides. The donor nucleic acid comprises a region that is complementary to a portion of the cellular target sequence (e.g., a homology arm). When optimally aligned, the donor nucleic acid overlaps with (is complementary to) the cellular target sequence by, e.g., about 20, 25, 30, 35, 40, 50, 60, 70, 80, 90 or more nucleotides. In many embodiments, the donor nucleic acid comprises two homology arms (regions complementary to the cellular target sequence) flanking the mutation or difference between the donor nucleic acid and the cellular target sequence. The donor nucleic acid comprises at least one mutation or alteration compared to the cellular target sequence, such as an insertion, deletion, modification, or any combination thereof compared to the cellular target sequence.

As described in relation to the gRNA, the donor nucleic acid is preferably provided as part of a rationally-designed editing cassette, which is inserted into an editing plasmid backbone (in yeast, preferably a linear plasmid backbone) where the editing plasmid backbone may comprise a promoter to drive transcription of the editing gRNA and the donor DNA when the editing cassette is inserted into the editing plasmid backbone. Moreover, there may be more than one, e.g., two, three, four, or more editing gRNA/donor nucleic acid rationally-designed editing cassettes inserted into an editing vector; alternatively, a single rationally-designed editing cassette may comprise two to several editing gRNA/donor DNA pairs, where each editing gRNA is under the control of separate different promoters, separate like promoters, or where all gRNAs/donor nucleic acid pairs are under the control of a single promoter. In some embodiments the promoter driving transcription of the editing gRNA and the donor nucleic acid (or driving more than one editing gRNA/donor nucleic acid pair) is optionally an inducible promoter.

In addition to the donor nucleic acid, an editing cassette may comprise one or more primer sites. The primer sites can be used to amplify the editing cassette by using oligonucleotide primers; for example, if the primer sites flank one or more of the other components of the editing cassette. In addition, the editing cassette may comprise a barcode. A barcode is a unique DNA sequence that corresponds to the donor DNA sequence such that the barcode can identify the edit made to the corresponding cellular target sequence. The barcode typically comprises four or more nucleotides. In some embodiments, the editing cassettes comprise a collection or library editing gRNAs and of donor nucleic acids representing, e.g., gene-wide or genome-wide libraries of editing gRNAs and donor nucleic acids. The library of editing cassettes is cloned into vector backbones where, e.g., each different donor nucleic acid is associated with a different barcode. Also, in preferred embodiments, an editing vector or plasmid encoding components of the nucleic acid-guided nuclease system further encodes a nucleic acid-guided nuclease comprising one or more nuclear localization sequences (NLSs), such as about or more than about 1, 2, 3, 4, 5, 6, 7, 8, 9, 10, or more NLSs, particularly as an element of the nuclease sequence. In some embodiments, the engineered nuclease comprises NLSs at or near the amino-terminus, NLSs at or near the carboxy-terminus, or a combination.

### Increasing Efficiency of Editing

The present disclosure is drawn to increasing the efficiency of nucleic acid-guided nuclease editing. Genome editing using nucleic acid-guided nuclease editing technology requires precise repair of nuclease-induced double-strand DNA breaks via homologous recombination with an editing (e.g., donor) plasmid. Double-strand DNA breaks in cells caused by nucleic acid-guided nucleases have three main outcomes: 1) cell death if the break is not repaired; 2) non-homologous end joining (NHEJ), which repairs the break without a homologous repair template; and 3) homologous recombination (HR), which uses auxiliary (here, exogenous) homologous DNA (e.g., a donor DNA sequence from an editing cassette inserted into the editing plasmid) to repair the break.

To increase HR in nucleic-guided nuclease editing, a split nuclease transcription factor tethering system is employed. The split nuclease transcription factor tethering system comprises two nuclease fusion constructs and an editing vector comprising a transcription factor binding site. The first nuclease fusion construct comprises an N-terminal portion of a nuclease linked to a first transcription factor (e.g., an N-terminal transcription factor construct), and the second nuclease fusion construct comprises a C-terminal portion of a nuclease linked to a second transcription factor (e.g., a C-terminal transcription factor construct), where the first and second transcription factors dimerize in the presence of a ligand. The first and second transcription factors may be the same or the first and second transcriptions factors may be different as described below; however, the first and second transcription factors must be able to dimerize in the presence of a ligand. The editing vector comprises at least one gRNA and donor DNA pair (e.g., a CREATE cassette) and a binding site transcription factor.

Once dimerized, the transcription factor components of the N-terminal and C-terminal transcription factor constructs bind to the transcription factor binding site on the editing vector, which results in 1) bringing the N-terminal and C-terminal portions of the nuclease into proximity resulting in a nuclease with “reconstituted” activity that is able to bind to a target nucleic acid in a cell, and 2) tethering the editing vector to the target nucleic acid in the cell. This process increases the efficiency of nucleic acid-guided editing by both allowing for control over the timing of the editing process in the cells and by physically tethering the target site for the edit to the editing vector thereby increasing the local concentration of donor DNA for templating homologous repair (HR). Despite precedent for split nuclease enzyme functionality, other embodiments have focused on the use of small molecule inducers but fail to provide a logic that that spatially links the HR donor repair substrate to the site of the dsDNA break in order to promote high efficiency precision editing. The systems described herein thus provide a general means to control both the temporal and spatial induction of homologous repair for such genome editing workflows. The recruitment of the editing plasmid to the target nucleic acid (e.g., the site of the double strand break) via the nuclease/transcription factor fusion constructs significantly increases editing rates in a multiplex library format. Further, initiation of the editing process is dependent on the dimerization of the transcription factor components, which in turn is dependent upon addition of an appropriate ligand. Thus, the editing process can be tightly controlled leading to, e.g., increased cell survival.

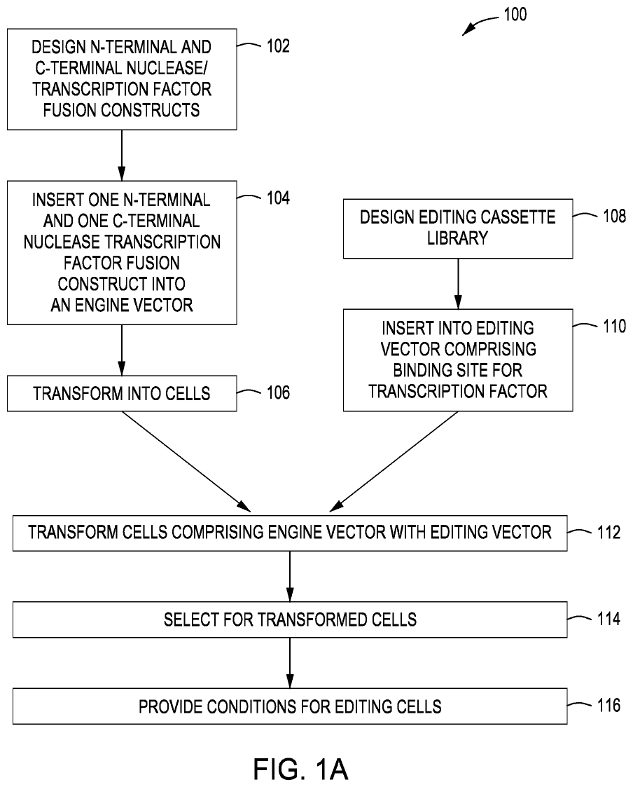

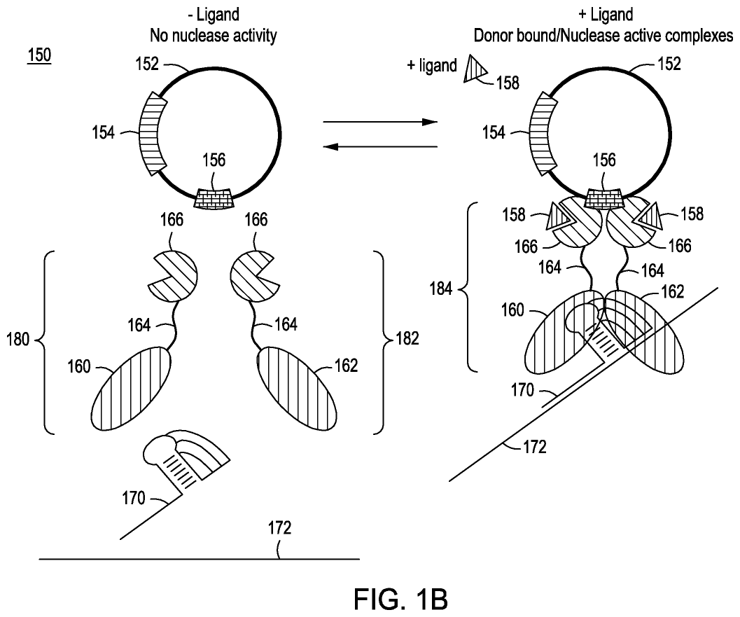

FIG. 1A is a simple process diagram for nucleic acid-guided nuclease editing using an engine vector comprising an N-terminal transcription factor construct and a C-terminal transcription factor construct where each of the N-terminal nuclease portion and C-terminal nuclease portion is attached via a linker to a transcription factor. In a first step of method 100, N-terminal and C-terminal transcription factor constructs are synthesized 102. The appropriate “split” between the N-terminal and C-terminal portions of the nuclease to produce a reconstituted nuclease once the two portions are in proximity with one another may be determined empirically for a desired nuclease as described below in Example 2. The nuclease is “split” at a point where there is no spontaneous association between the N-terminal and C-terminal portions of the nuclease in the absence of a ligand (e.g., the ligand that causes the transcription factors to dimerize) and in the absence of a transcription factor binding site on an editing vector; however, the “split” must allow association of the N-terminal and C-terminal portions of the nuclease—and reconstitution of nuclease activity—in the presence of the ligand and binding of the dimerized transcription factor at the transcription factor binding site. Once the proper “split” for the nuclease is determined, appropriate N-terminal and C-terminal transcription factor constructs can be designed and synthesized for the desired nuclease.

In molecular biology, a transcription factor (or sequence-specific DNA-binding factor) is a protein that controls the rate of transcription of genetic information from DNA to messenger RNA, by binding to a specific DNA sequence. The function of transcription factors is to regulate—turn on and off—genes in order to make sure that the genes are expressed in the right cell at the right time and in the right amount throughout the life of the cell and the organism. Groups of transcription factors function in a coordinated fashion to direct cell division, cell growth, and cell death throughout life; to direct cell migration and organization during embryonic development; and intermittently to respond to signals from outside the cell, such as a hormone. A defining feature of transcription factors is that they contain at least one DNA-binding domain, which attaches to a specific sequence of DNA adjacent to the genes that they regulate.

In many transcription factor gene families, proteins require a physical interaction with small molecule or peptide ligand to initiate conformational changes that lead to DNA binding. Ligand binding leads to homodimerization or heterodimerization depending on the identity of the transcription factor. In nature, depending on the choice of partner and the cellular context, each dimer triggers a sequence of regulatory events that lead to a particular cellular fate, for example, proliferation or differentiation. Recent syntheses of genomic and functional data reveal that partner choice is not random; instead, dimerization specificities, which are strongly linked to the evolution of the protein family, apply. The present compositions and methods take advantage of this required dimerization to regulate nuclease activity. Dimeric transcription factors that bind to DNA are often grouped into families on the basis of dimerization and DNA-binding specificities. cDNA cloning studies have established that members of the same family have structurally related dimerization and DNA-binding domains but diverge in other regions that are important for transcriptional activation. These features lead to the straightforward suggestion that although all members of a family bind to similar DNA elements, individual members exhibit distinct transcriptional effector functions.

Conserved DNA binding motifs are present among DNA binding proteins. These include domains known as the leucine zipper, the helix-turn-helix, the zinc finger and the helix-loop helix domains. For example, the basic leucine zipper domain contains an alpha helix with a leucine at every 7th amino acid. If two leucine zipper domains find one another, the leucines can interact like the teeth in a zipper, allowing the dimerization of two proteins. When binding to the DNA, the leucine residues bind to the sugar-phosphate backbone while the helices sit in the major grooves. Table 1 lists exemplary transcription factors that function as dimers and thus would be appropriate for use in the N-terminal and C-terminal transcription factor constructs described herein.

TABLE 1
Examples of specific transcription factors

| | | | | SEQ |
|---|---|---|---|---|
| | Structural | Recognition | Binds | ID |
| Factor | Type | sequence | as | No. |
| Ara^{R} | Winged helix- | TAATATTGTACG | Dimer | 1 |
| | turn-helix | AACAATTT | | |
| Xln^{R} | Winged helix- | TTAGSCTAA | Dimer | |
| | turn-helix | | | |
| AP-1 | Basic zipper | 5′-TGA(G/C)T | Dimer | |
| (activator | | CA-3′ | | |
| protein 1) | | | | |
| C/EBP | Basic zipper | 5′-ATTGCGCAA | Dimer | 2 |
| (CCAAT- | | T-3 | | |
| enhancer | | | | |
| binding | | | | |
| protein | | | | |
| ATF/CREB | Basic zipper | 5′-TGACGTC | Dimer | |
| activating | | A-3′ | | |
| transcription | | | | |
| factor/cAMP | | | | |
| response | | | | |
| binding | | | | |
| element | | | | |
| c-Myc | Basic helix- | 5′-CACGTG-3′ | Dimer | |
| | loop-helix | | | |
| NF-1 nuclear | Novel | 5′-TTGGCNNNN | Dimer | 3 |
| factor 1 | | NGCCAA-3′ | | |

Once synthesized, one of each of an N-terminal transcription factor construct and a C-terminal transcription factor construct is inserted into an engine vector 104 to be transformed 106 into cells of choice such that they are co-expressed within the cells that harbor this construct. The N-terminal TF construct and the C-terminal TF constructs may be contained in a single vector and under the control of the same promoter, the N-terminal TF construct and the C-terminal TF constructs may be contained in a single vector and under the control of different promoters. Alternatively, the N-terminal TF construct and the C-terminal TF constructs may reside on separate engine vectors and the cells may be transformed simultaneously or sequentially with the separate engine vectors. In yet another alternative, the one or both of the N-terminal transcription factor construct and the C-terminal transcription factor construct may be stably integrated into the cellular genome.

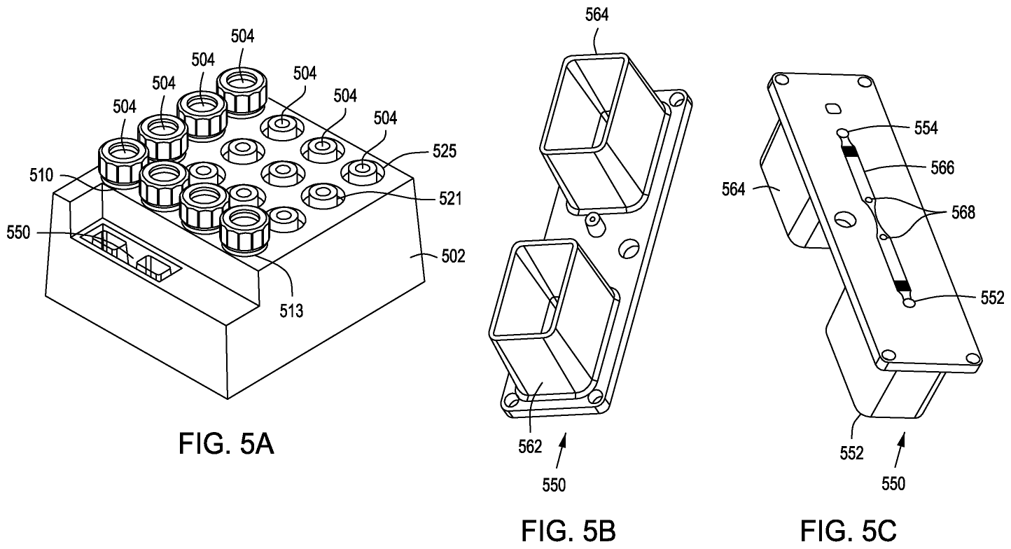

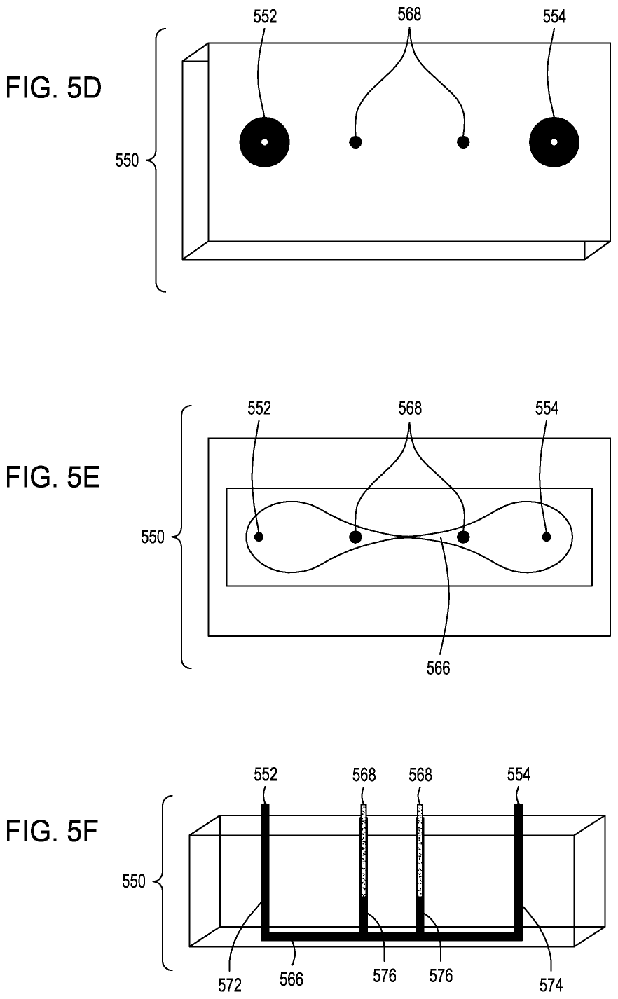

Transformation is intended to include to a variety of art-recognized techniques for introducing an exogenous nucleic acid sequence (e.g., engine and/or editing vectors) into a target cell, and the term “transformation” as used herein includes all transformation and transfection techniques. Such methods include, but are not limited to, electroporation, lipofection, optoporation, injection, microprecipitation, microinjection, liposomes, particle bombardment, sonoporation, laser-induced poration, bead transfection, calcium phosphate or calcium chloride co-precipitation, or DEAE-dextran-mediated transfection. Cells can also be prepared for vector uptake using, e.g., a sucrose, sorbitol or glycerol wash. Additionally, hybrid techniques that exploit the capabilities of mechanical and chemical transfection methods can be used, e.g., magnetofection, a transfection methodology that combines chemical transfection with mechanical methods. In another example, cationic lipids may be deployed in combination with gene guns or electroporators. Suitable materials and methods for transforming or transfecting target cells can be found, e.g., in Green and Sambrook, Molecular Cloning: A Laboratory Manual, 4th, ed., Cold Spring Harbor Laboratory Press, Cold Spring Harbor, N.Y., 2014). The present automated methods using the automated multi-module cell processing instrument utilize flow-through electroporation such as the exemplary device shown in FIGS. 5B-5F.

Simultaneously or next, an editing cassette library is designed 108. Methods and compositions for designing and synthesizing editing cassettes are described in U.S. Ser. Nos. 10,240,167; 10,266,849; 9,982,278; 10,351,877; and 10,364,442; and U.S. Ser. No. 16/275,439, filed 14 Feb. 2019; and Ser. No. 16/275,465, filed 14 Feb. 2019. U.S. Ser. No. 16/275,465, filed 14 Feb. 2019 describes compound editing cassettes that are used in some embodiments of the compositions and methods described herein. Compound editing cassettes are editing cassettes comprising more than one gRNA and more than one donor DNA. Once designed 108 and synthesized, the library of editing cassettes is amplified, purified and inserted 110 into an editing vector to produce a library of editing vectors where the editing vectors also comprise a binding site for the dimerized transcription factor linked to the N-terminal and C-terminal portions of the nuclease. The library of editing vectors is then transformed into the cells that have already been transformed with the N-terminal and C-terminal transcription factor constructs 112.

Once transformed, the cells are allowed to recover and selection is performed 114 to select for cells transformed with the engine vector(s) and editing vector, both of which most often comprise a selectable marker. As described above, drug selectable markers such as ampicillin/carbenicillin, kanamycin, chloramphenicol, nourseothricin N-acetyl transferase, erythromycin, tetracycline, gentamicin, bleomycin, streptomycin, puromycin, hygromycin, blasticidin, and G418 or other selectable markers may be employed. At a next step, conditions are provided such that editing takes place 116, and the cells may be used in research or may be grown to a desired OD to be made electrocompetent again, followed by another round of editing.

FIG. 1B is a simplified schematic 150 of the components of a split nuclease tethering system used to perform nucleic acid-guided nuclease editing. At left in the figure is an editing vector 152 comprising a CREATE cassette 154 (sequences for a gRNA and a donor DNA to be transcribed) and a binding site for a transcription factor 156. Also seen is ligand 158. Below editing vector 152 is at left, an N-terminal transcription factor construct 180 comprising an N-terminal portion of a nuclease 160, a linker 164, and a transcription factor 166. Below editing vector 152 at right is a C-terminal transcription factor construct 182 comprising a C-terminal portion of a nuclease 162, a linker 164, and a transcription factor 166. Note that in this exemplary schematic, linkers 164 are the same; in other embodiments, however, the linkers connecting the N-terminal and C-terminal portions of the nuclease to the transcription factors may be different.

Linkers typically are short peptide sequences often composed of flexible amino acid residues like glycine and serine so that the N-terminal and C-terminal nuclease domains and the transcription factor domains are free to move relative to one another; that is, to allow the N-terminal and C-terminal domains to associate with one another to reconstitute nuclease activity and to allow the transcription factors to dimerize. Exemplary linkers include (GGGGS)₃, G₈, and (EAAAK)₃.

As with linkers 164, note that in this exemplary schematic, transcription factors 166 are the same; however in other embodiments the transcription factor connected to the N-terminal and C-terminal portions of the nuclease may be different with the caveat that the two transcription factors 166 must be able to dimerize and bind the transcription factor binding site 156 located on editing vector 152. At left, in the absence of ligand 158 the N-terminal 180 and C-terminal 182 transcription factor constructs are not in proximity to one another; that is, transcription factors 166 are not dimerized and are not coupled together; thus the N-terminal 180 and C-terminal 182 transcription factor constructs are not brought into proximity and the N-terminal 160 and C-terminal 162 portions of the nuclease do not associate and cannot reconstitute nuclease activity. Also seen in FIG. 1B is gRNA/donor DNA transcript 170 and target nucleic acid 172.

With the addition of ligand 158, however, transcription factors 166 dimerize, bind to transcription factor binding site 156 and bring the N-terminal 160 and C-terminal 162 portions of the nuclease into proximity 184 resulting in a nuclease with reconstituted activity. The reconstituted nuclease is able to bind both the gRNA/donor DNA transcript 170 and target nucleic acid 172 allowing for editing. Tethering the editing vector 152 to the target nucleic acid 172 brings the target nucleic acid proximal to the gRNA/donor DNA transcripts 170 which are being actively transcribed from editing vector 152.

### Automated Cell Editing Instruments and Modules to Perform Nucleic Acid-Guided Nuclease Editing in Cells

### Automated Cell Editing Instruments

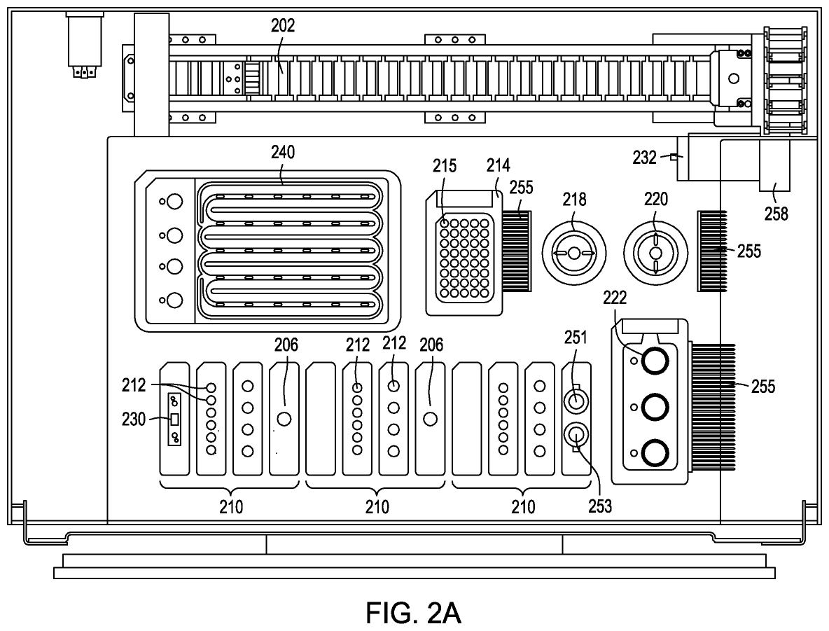

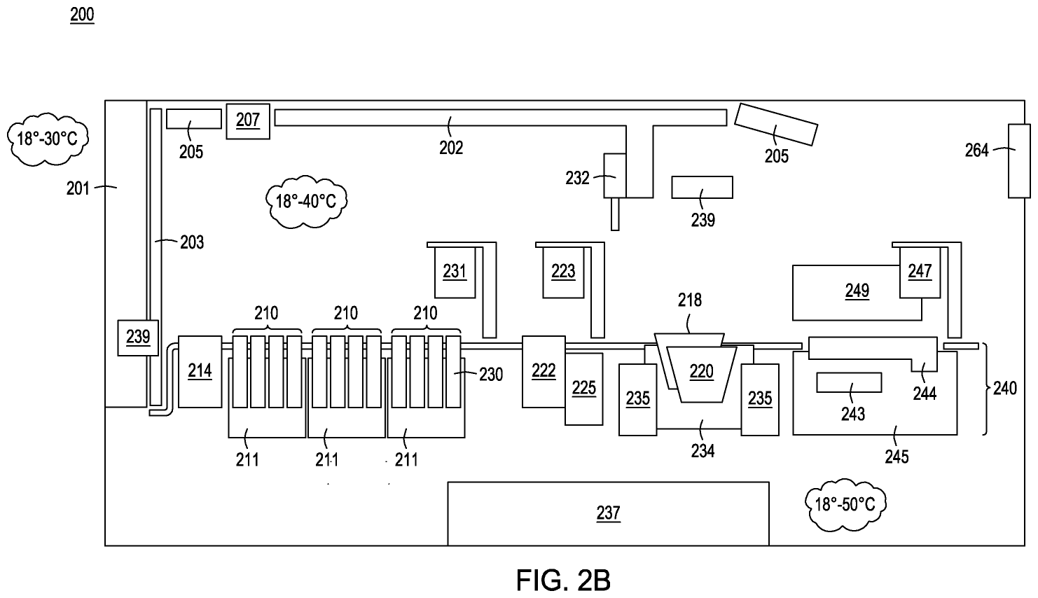

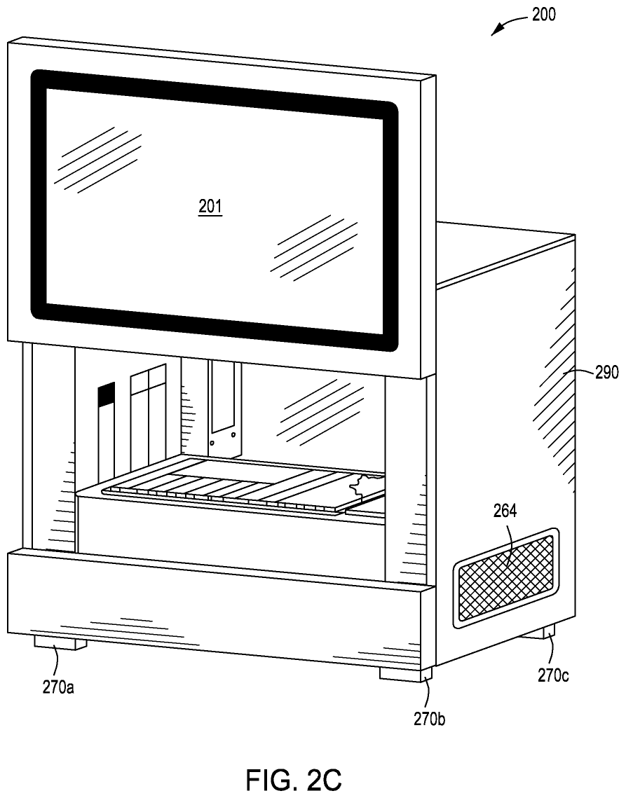

FIG. 2A depicts an exemplary automated multi-module cell processing instrument 200 to, e.g., perform one of the exemplary workflows comprising a split nuclease transcription factor tethering system as described herein. The instrument 200, for example, may be and preferably is designed as a stand-alone desktop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (i.e., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other embodiments, the liquid handling system 258 may stay stationary while the various modules and reagent cartridges are moved. Also included in the automated multi-module cell processing instrument 200 are reagent cartridges 210 comprising reservoirs 212 and transformation module 230 (e.g., a flow-through electroporation device as described in detail in relation to FIGS. 5B-5F), as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge 210 and wash cartridge 204 may be identical except for the consumables (reagents or other components contained within the various inserts) inserted therein.

In some implementations, the reagent cartridges 210 are disposable kits comprising reagents and cells for use in the automated multi-module cell processing/editing instrument 200. For example, a user may open and position each of the reagent cartridges 210 comprising various desired inserts and reagents within the chassis of the automated multi-module cell editing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, Nev. (see, e.g., WO2018015544A1), or Beckman Coulter, Inc. of Fort Collins, Colo. (see, e.g., US20160018427A1). Pipette tips may be provided in a pipette transfer tip supply (not shown) for use with the air displacement pipettor 232.

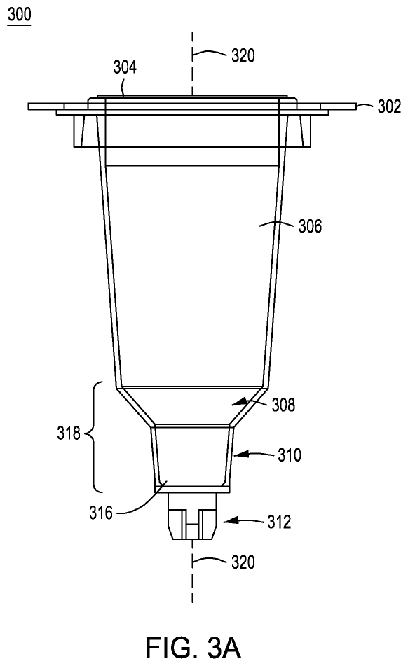

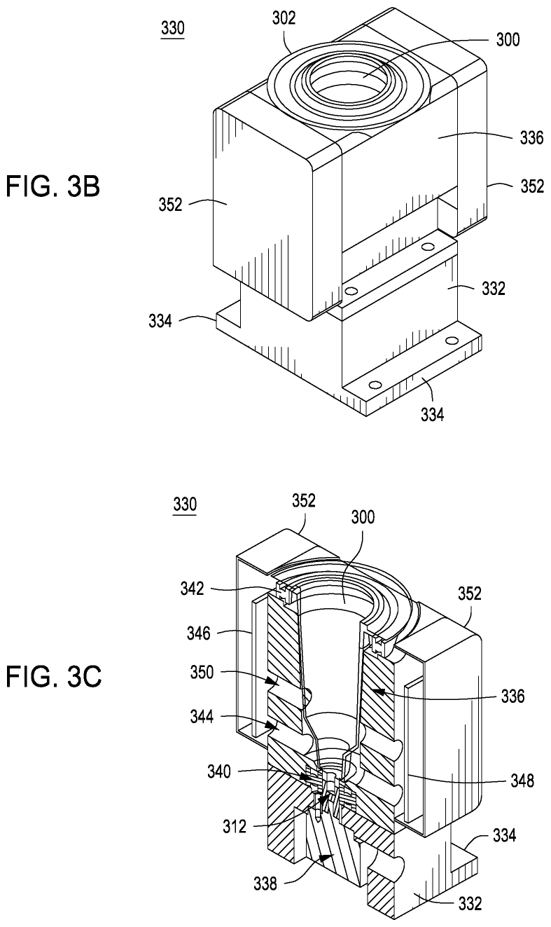

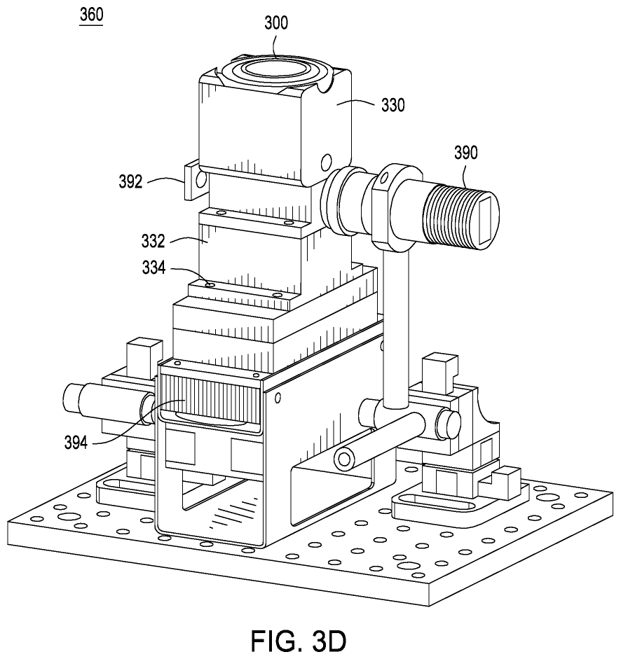

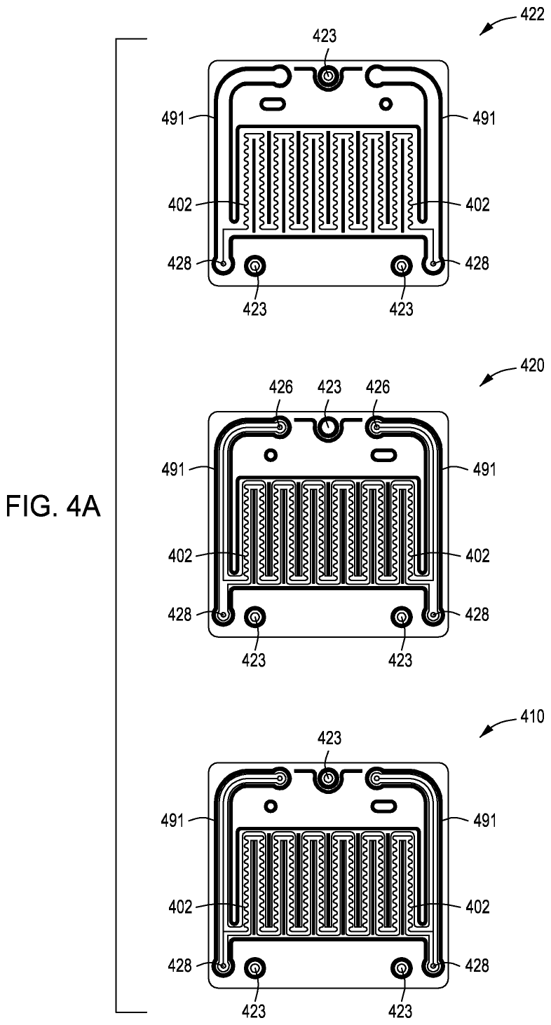

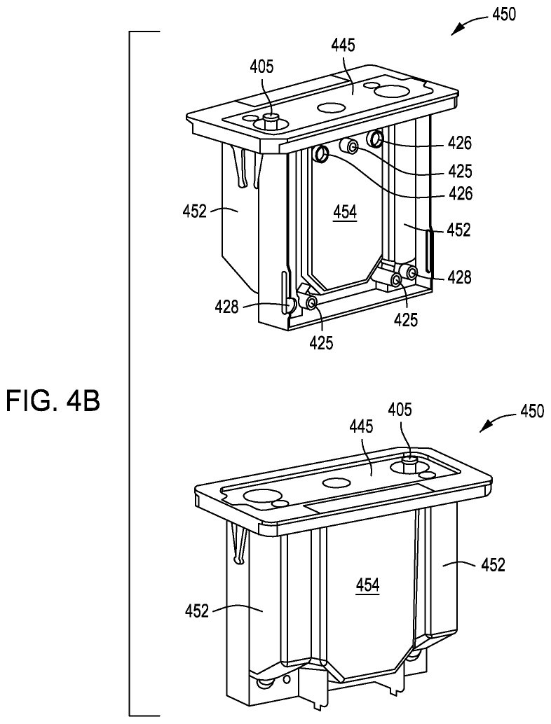

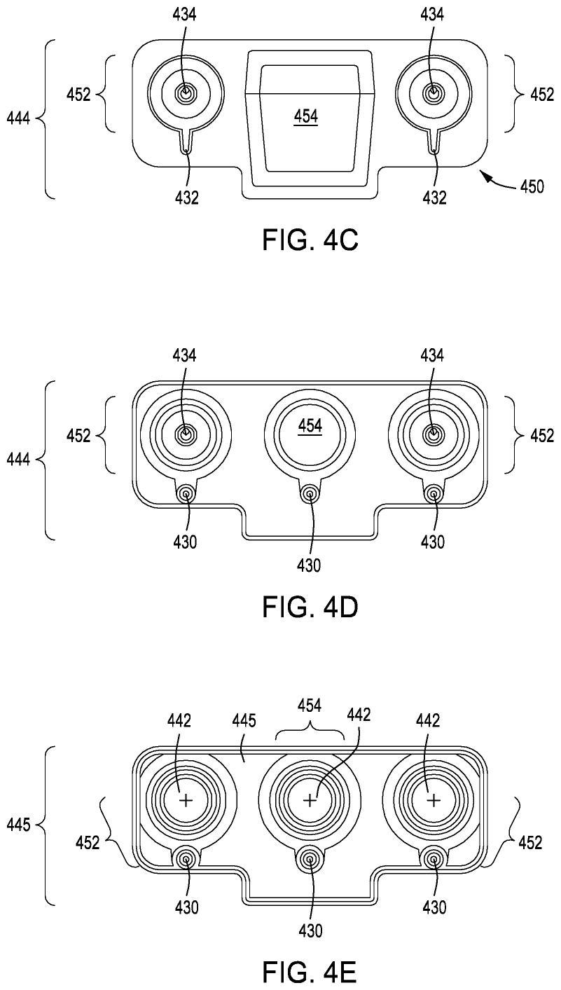

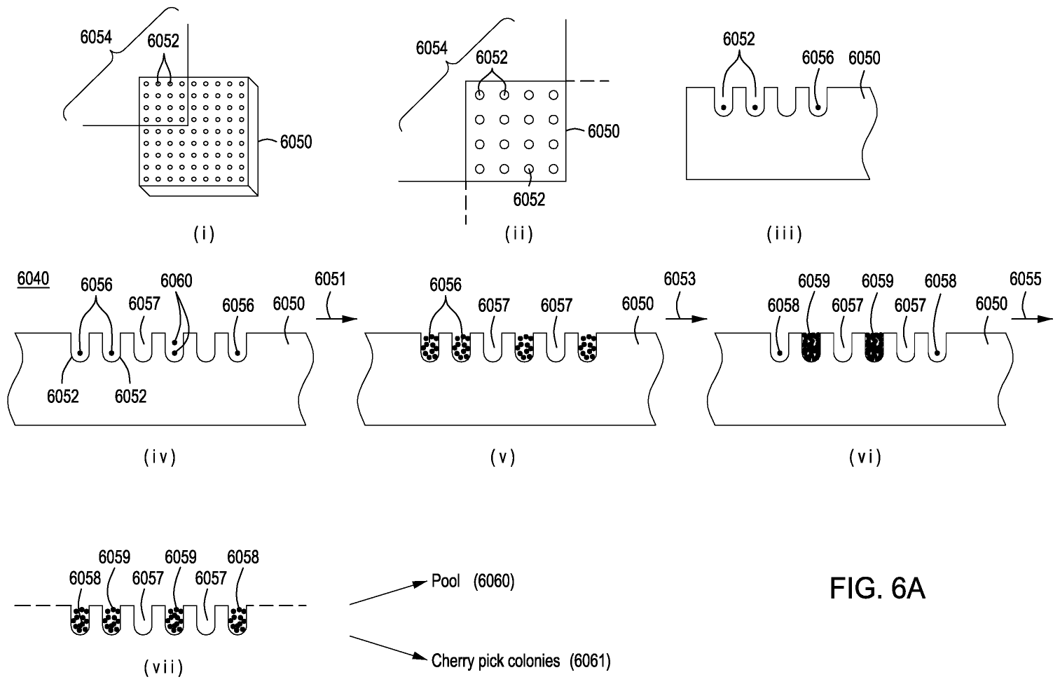

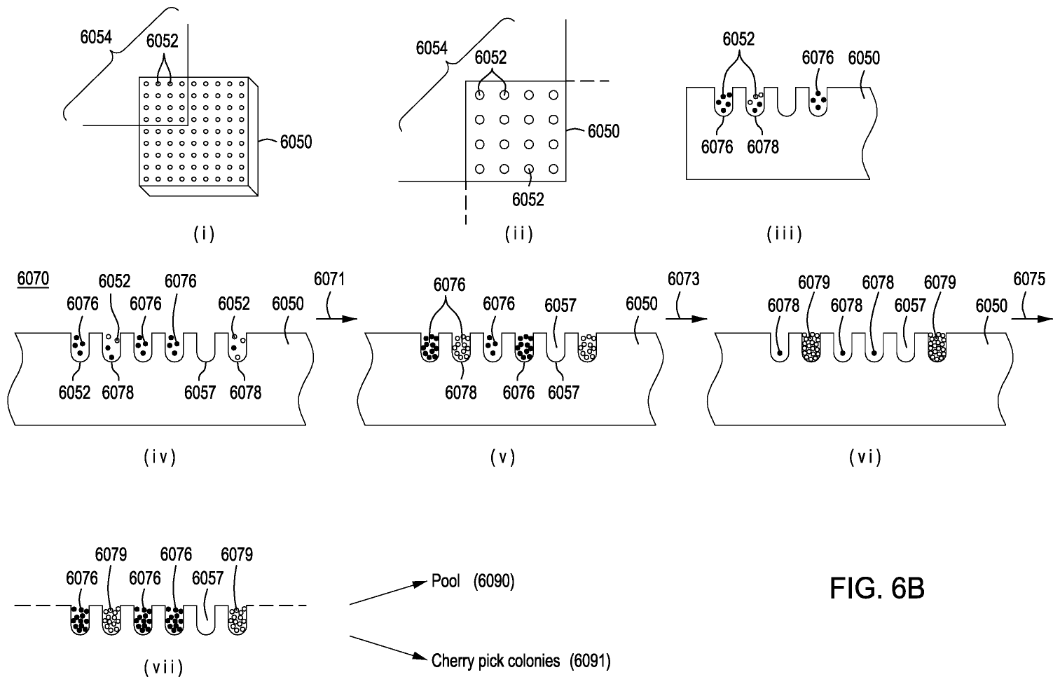

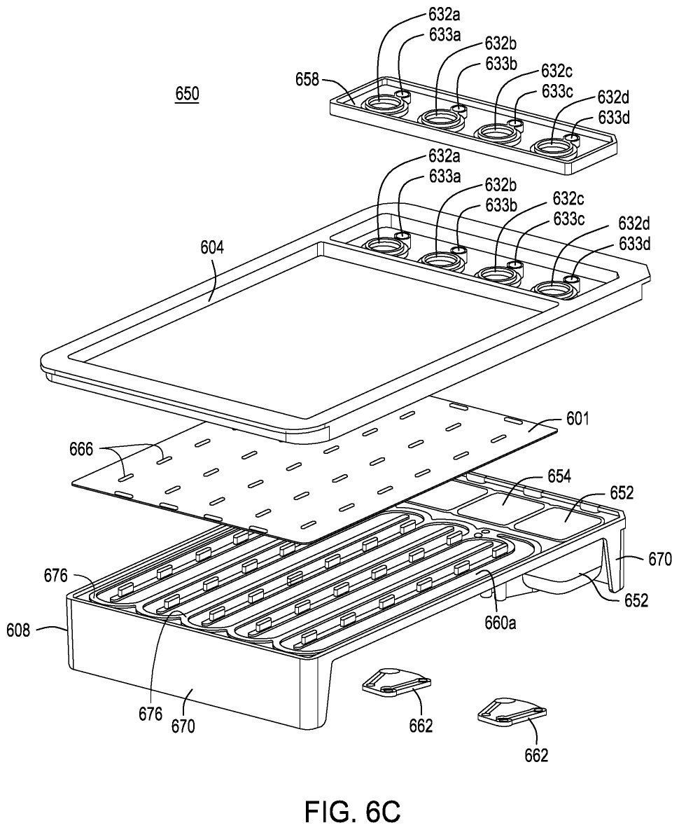

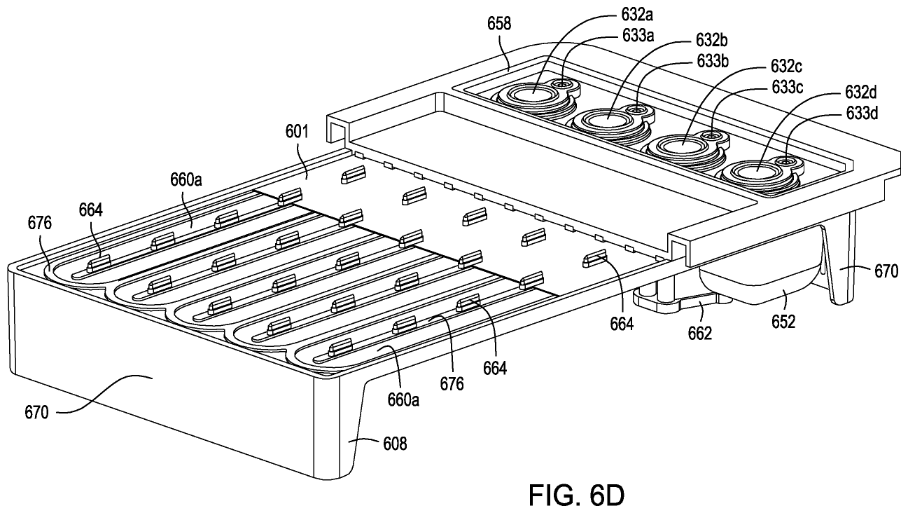

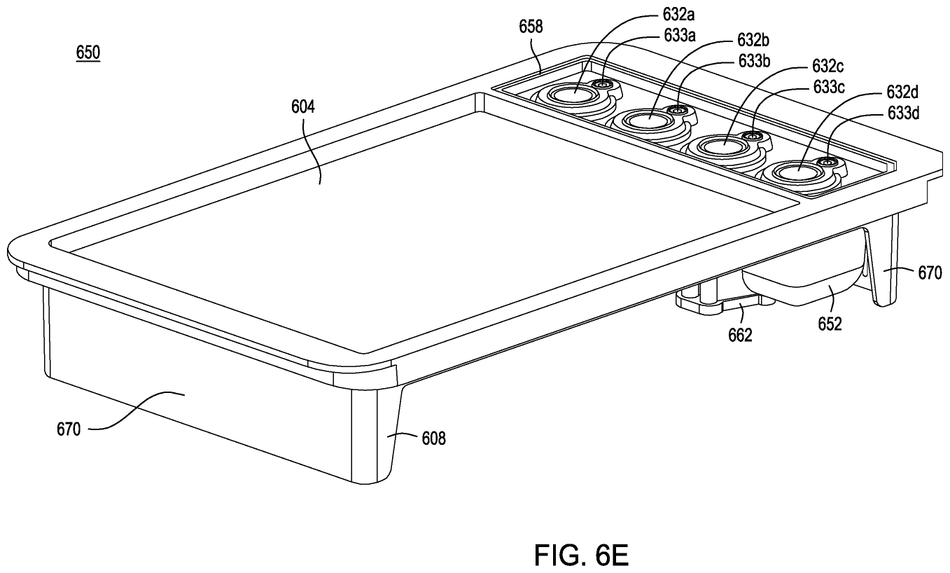

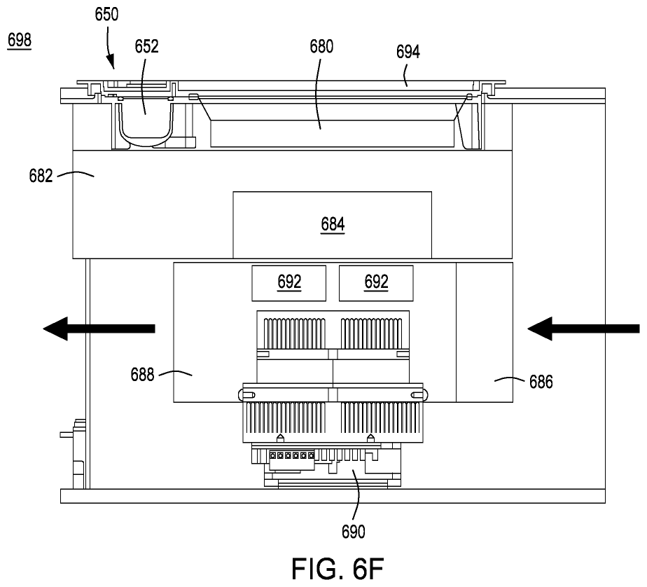

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell editing instrument 200 may identify a stored materials map based upon the machine-readable indicia. In the embodiment illustrated in FIG. 2A, a cell growth module comprises two cell growth vials 218, 220 (described in greater detail below in relation to FIGS. 3A-3D). Additionally seen is the TFF module 222 (described above in detail in relation to FIGS. 4A-aE). Also illustrated as part of the automated multi-module cell processing instrument 200 of FIG. 2A is a singulation module 240 (e.g., a solid wall isolation, incubation and normalization device (SWIIN device) is shown here) described herein in relation to FIGS. 6C-6F, served by, e.g., robotic liquid handing system 258 and air displacement pipettor 232. Also note the placement of three heatsinks 255.

FIG. 2B is a simplified representation of the contents of the exemplary multi-module cell processing instrument 200 depicted in FIG. 2A. Cartridge-based source materials (such as in reagent cartridges 210), for example, may be positioned in designated areas on a deck of the instrument 200 for access by an air displacement pipettor 232. The deck of the multi-module cell processing instrument 200 may include a protection sink such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 233. Thermal assemblies 225, 235, and 245 encompass thermal electric devices such as Peltier devices, as well as heatsinks, fans and coolers. The two rotating growth vials 218 and 220 are within a growth module 234, where the growth module is served by two thermal assemblies 235. Also seen is the SWIIN module 240, comprising a SWIIN cartridge 241, where the SWIIN module also comprises a thermal assembly 245, illumination 243 (in this embodiment, backlighting), evaporation and condensation control 249, and where the SWIIN module is served by SWIIN interface (e.g., manifold arm) and actuator 247. Also seen in this view is touch screen display 201, display actuator 203, illumination 205 (one on either side of multi-module cell processing instrument 200), and cameras 239 (one illumination device on either side of multi-module cell processing instrument 200). Finally, element 237 comprises electronics, such as circuit control boards, high-voltage amplifiers, power supplies, and power entry; as well as pneumatics, such as pumps, valves and sensors.

FIGS. 2C through 2E illustrate front perspective (door open), side perspective, and front perspective (door closed) views, respectively, of multi-module cell processing instrument 200 for use in as a desktop version of the automated multi-module cell editing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell editing instrument 200 and accepts inputs from the user for conducting the cell processing. In this embodiment, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device, one or more rotating growth vials 218, 220 in a cell growth module 234, a tangential flow filtration module 222, a SWIIN module 240 as well as interfaces and actuators for the various modules. In addition, chassis 290 houses control circuitry, liquid handling tubes, air pump controls, valves, sensors, thermal assemblies (e.g., heating and cooling units) and other control mechanisms. FIG. 2C is a side perspective view of automated multi-module cell editing instrument 200, showing chassis 290, touch screen display 201, adjustable feel 270b, 270c, and 270d, and cooling grates 264. FIG. 2D is a front perspective view of automated multi-module cell editing instrument 200 with the touch screen display (e.g., front door) 201 closed. Again seen are chassis 290, cooling grate 264, and adjustable feet 270a, 270b and 270c. For examples of multi-module cell editing instruments, see U.S. Ser. No. 10,253,316, issued 9 Apr. 2019; U.S. Pat. No. 10,329,559, issued 25 Jun. 2019; and U.S. Pat. No. 10,323,242, issued 18 Jun. 2019; and U.S. Ser. No. 16/412,175, filed 14 May 2019; Ser. No. 16/412,195, filed 14 May 2019; and Ser. No. 16/423,289, filed 28 May 2019, all of which are herein incorporated by reference in their entirety.

### The Rotating Cell Growth Module

FIG. 3A shows one embodiment of a rotating growth vial 300 for use with the cell growth device and in the automated multi-module cell processing instruments described herein. The rotating growth vial 300 is an optically-transparent container having an open end 304 for receiving liquid media and cells, a central vial region 306 that defines the primary container for growing cells, a tapered-to-constricted region 318 defining at least one light path 310, a closed end 316, and a drive engagement mechanism 312. The rotating growth vial 300 has a central longitudinal axis 320 around which the vial rotates, and the light path 310 is generally perpendicular to the longitudinal axis of the vial. The first light path 310 is positioned in the lower constricted portion of the tapered-to-constricted region 318. Optionally, some embodiments of the rotating growth vial 300 have a second light path 308 in the tapered region of the tapered-to-constricted region 318. Both light paths in this embodiment are positioned in a region of the rotating growth vial that is constantly filled with the cell culture (cells+growth media) and are not affected by the rotational speed of the growth vial. The first light path 310 is shorter than the second light path 308 allowing for sensitive measurement of OD values when the OD values of the cell culture in the vial are at a high level (e.g., later in the cell growth process), whereas the second light path 308 allows for sensitive measurement of OD values when the OD values of the cell culture in the vial are at a lower level (e.g., earlier in the cell growth process).

The drive engagement mechanism 312 engages with a motor (not shown) to rotate the vial. In some embodiments, the motor drives the drive engagement mechanism 312 such that the rotating growth vial 300 is rotated in one direction only, and in other embodiments, the rotating growth vial 300 is rotated in a first direction for a first amount of time or periodicity, rotated in a second direction (i.e., the opposite direction) for a second amount of time or periodicity, and this process may be repeated so that the rotating growth vial 300 (and the cell culture contents) are subjected to an oscillating motion. Further, the choice of whether the culture is subjected to oscillation and the periodicity therefor may be selected by the user. The first amount of time and the second amount of time may be the same or may be different. The amount of time may be 1, 2, 3, 4, 5, or more seconds, or may be 1, 2, 3, 4 or more minutes. In another embodiment, in an early stage of cell growth the rotating growth vial 400 may be oscillated at a first periodicity (e.g., every 60 seconds), and then a later stage of cell growth the rotating growth vial 300 may be oscillated at a second periodicity (e.g., every one second) different from the first periodicity.

The rotating growth vial 300 may be reusable or, preferably, the rotating growth vial is consumable. In some embodiments, the rotating growth vial is consumable and is presented to the user pre-filled with growth medium, where the vial is hermetically sealed at the open end 304 with a foil seal. A medium-filled rotating growth vial packaged in such a manner may be part of a kit for use with a stand-alone cell growth device or with a cell growth module that is part of an automated multi-module cell processing system. To introduce cells into the vial, a user need only pipette up a desired volume of cells and use the pipette tip to punch through the foil seal of the vial. Open end 304 may optionally include an extended lip 402 to overlap and engage with the cell growth device. In automated systems, the rotating growth vial 400 may be tagged with a barcode or other identifying means that can be read by a scanner or camera (not shown) that is part of the automated system.

The volume of the rotating growth vial 300 and the volume of the cell culture (including growth medium) may vary greatly, but the volume of the rotating growth vial 300 must be large enough to generate a specified total number of cells. In practice, the volume of the rotating growth vial 400 may range from 1-250 mL, 2-100 mL, from 5-80 mL, 10-50 mL, or from 12-35 mL. Likewise, the volume of the cell culture (cells+growth media) should be appropriate to allow proper aeration and mixing in the rotating growth vial 400. Proper aeration promotes uniform cellular respiration within the growth media. Thus, the volume of the cell culture should be approximately 5-85% of the volume of the growth vial or from 20-60% of the volume of the growth vial. For example, for a 30 mL growth vial, the volume of the cell culture would be from about 1.5 mL to about 26 mL, or from 6 mL to about 18 mL.

The rotating growth vial 300 preferably is fabricated from a bio-compatible optically transparent material—or at least the portion of the vial comprising the light path(s) is transparent. Additionally, material from which the rotating growth vial is fabricated should be able to be cooled to about 4° C. or lower and heated to about 55° C. or higher to accommodate both temperature-based cell assays and long-term storage at low temperatures. Further, the material that is used to fabricate the vial must be able to withstand temperatures up to 55° C. without deformation while spinning. Suitable materials include cyclic olefin copolymer (COC), glass, polyvinyl chloride, polyethylene, polyamide, polypropylene, polycarbonate, poly(methyl methacrylate (PMMA), polysulfone, polyurethane, and co-polymers of these and other polymers. Preferred materials include polypropylene, polycarbonate, or polystyrene. In some embodiments, the rotating growth vial is inexpensively fabricated by, e.g., injection molding or extrusion.

FIG. 3B is a perspective view of one embodiment of a cell growth device 330. FIG. 3C depicts a cut-away view of the cell growth device 330 from FIG. 3B. In both figures, the rotating growth vial 300 is seen positioned inside a main housing 336 with the extended lip 302 of the rotating growth vial 300 extending above the main housing 336. Additionally, end housings 352, a lower housing 332 and flanges 334 are indicated in both figures. Flanges 334 are used to attach the cell growth device 330 to heating/cooling means or other structure (not shown). FIG. 3C depicts additional detail. In FIG. 3C, upper bearing 342 and lower bearing 340 are shown positioned within main housing 336. Upper bearing 342 and lower bearing 340 support the vertical load of rotating growth vial 300. Lower housing 332 contains the drive motor 338. The cell growth device 330 of FIG. 3C comprises two light paths: a primary light path 344, and a secondary light path 350. Light path 344 corresponds to light path 310 positioned in the constricted portion of the tapered-to-constricted portion of the rotating growth vial 300, and light path 350 corresponds to light path 308 in the tapered portion of the tapered-to-constricted portion of the rotating growth via 316. Light paths 310 and 308 are not shown in FIG. 3C but may be seen in FIG. 3A. In addition to light paths 344 and 340, there is an emission board 348 to illuminate the light path(s), and detector board 346 to detect the light after the light travels through the cell culture liquid in the rotating growth vial 300.

The motor 338 engages with drive mechanism 312 and is used to rotate the rotating growth vial 300. In some embodiments, motor 338 is a brushless DC type drive motor with built-in drive controls that can be set to hold a constant revolution per minute (RPM) between 0 and about 3000 RPM. Alternatively, other motor types such as a stepper, servo, brushed DC, and the like can be used. Optionally, the motor 338 may also have direction control to allow reversing of the rotational direction, and a tachometer to sense and report actual RPM. The motor is controlled by a processor (not shown) according to, e.g., standard protocols programmed into the processor and/or user input, and the motor may be configured to vary RPM to cause axial precession of the cell culture thereby enhancing mixing, e.g., to prevent cell aggregation, increase aeration, and optimize cellular respiration.

Main housing 336, end housings 352 and lower housing 332 of the cell growth device 330 may be fabricated from any suitable, robust material including aluminum, stainless steel, and other thermally conductive materials, including plastics. These structures or portions thereof can be created through various techniques, e.g., metal fabrication, injection molding, creation of structural layers that are fused, etc. Whereas the rotating growth vial 300 is envisioned in some embodiments to be reusable, but preferably is consumable, the other components of the cell growth device 330 are preferably reusable and function as a stand-alone benchtop device or as a module in a multi-module cell processing system.

The processor (not shown) of the cell growth device 330 may be programmed with information to be used as a “blank” or control for the growing cell culture. A “blank” or control is a vessel containing cell growth medium only, which yields 100% transmittance and 0 OD, while the cell sample will deflect light rays and will have a lower percent transmittance and higher OD. As the cells grow in the media and become denser, transmittance will decrease and OD will increase. The processor (not shown) of the cell growth device 330—may be programmed to use wavelength values for blanks commensurate with the growth media typically used in cell culture (whether, e.g., mammalian cells, bacterial cells, animal cells, yeast cells, etc.). Alternatively, a second spectrophotometer and vessel may be included in the cell growth device 330, where the second spectrophotometer is used to read a blank at designated intervals.

FIG. 3D illustrates a cell growth device 330 as part of an assembly comprising the cell growth device 330 of FIG. 3B coupled to light source 390, detector 392, and thermal components 394. The rotating growth vial 300 is inserted into the cell growth device. Components of the light source 390 and detector 392 (e.g., such as a photodiode with gain control to cover 5-log) are coupled to the main housing of the cell growth device. The lower housing 332 that houses the motor that rotates the rotating growth vial 300 is illustrated, as is one of the flanges 334 that secures the cell growth device 330 to the assembly. Also, the thermal components 394 illustrated are a Peltier device or thermoelectric cooler. In this embodiment, thermal control is accomplished by attachment and electrical integration of the cell growth device 330 to the thermal components 394 via the flange 334 on the base of the lower housing 332. Thermoelectric coolers are capable of “pumping” heat to either side of a junction, either cooling a surface or heating a surface depending on the direction of current flow. In one embodiment, a thermistor is used to measure the temperature of the main housing and then, through a standard electronic proportional-integral-derivative (PID) controller loop, the rotating growth vial 300 is controlled to approximately +/−0.5° C.

In use, cells are inoculated (cells can be pipetted, e.g., from an automated liquid handling system or by a user) into pre-filled growth media of a rotating growth vial 300 by piercing though the foil seal or film. The programmed software of the cell growth device 330 sets the control temperature for growth, typically 30° C., then slowly starts the rotation of the rotating growth vial 300. The cell/growth media mixture slowly moves vertically up the wall due to centrifugal force allowing the rotating growth vial 300 to expose a large surface area of the mixture to a normal oxygen environment. The growth monitoring system takes either continuous readings of the OD or OD measurements at pre-set or pre-programmed time intervals. These measurements are stored in internal memory and if requested the software plots the measurements versus time to display a growth curve. If enhanced mixing is required, e.g., to optimize growth conditions, the speed of the vial rotation can be varied to cause an axial precession of the liquid, and/or a complete directional change can be performed at programmed intervals. The growth monitoring can be programmed to automatically terminate the growth stage at a pre-determined OD, and then quickly cool the mixture to a lower temperature to inhibit further growth.

One application for the cell growth device 330 is to constantly measure the optical density of a growing cell culture. One advantage of the described cell growth device is that optical density can be measured continuously (kinetic monitoring) or at specific time intervals; e.g., every 5, 10, 15, 20, 30 45, or 60 seconds, or every 1, 2, 3, 4, 5, 6, 7, 8, 9, or 10 minutes. While the cell growth device 330 has been described in the context of measuring the optical density (OD) of a growing cell culture, it should, however, be understood by a skilled artisan given the teachings of the present specification that other cell growth parameters can be measured in addition to or instead of cell culture OD. As with optional measure of cell growth in relation to the solid wall device or module described supra, spectroscopy using visible, UV, or near infrared (NIR) light allows monitoring the concentration of nutrients and/or wastes in the cell culture and other spectroscopic measurements may be made; that is, other spectral properties can be measured via, e.g., dielectric impedance spectroscopy, visible fluorescence, fluorescence polarization, or luminescence. Additionally, the cell growth device 430 may include additional sensors for measuring, e.g., dissolved oxygen, carbon dioxide, pH, conductivity, and the like. For additional details regarding rotating growth vials and cell growth devices see U.S. Ser. No. 16/360,404, filed 21 Mar. 2019 and Ser. No. 16/360,423, filed 21 Mar. 2019.

### The Cell Concentration Module

As described above in relation to the rotating growth vial and cell growth module, in order to obtain an adequate number of cells for transformation or transfection, cells typically are grown to a specific optical density in medium appropriate for the growth of the cells of interest; however, for effective transformation or transfection, it is desirable to decrease the volume of the cells as well as render the cells competent via buffer or medium exchange. Thus, one sub-component or module that is desired in cell processing systems for the processes listed above is a module or component that can grow, perform buffer exchange, and/or concentrate cells and render them competent so that they may be transformed or transfected with the nucleic acids needed for engineering or editing the cell's genome.

FIG. 4A shows a retentate member 422 (top), permeate member 420 (middle) and a tangential flow assembly 410 (bottom) comprising the retentate member 422, membrane 424 (not seen in FIG. 4A), and permeate member 420 (also not seen). In FIG. 4A, retentate member 422 comprises a tangential flow channel 402, which has a serpentine configuration that initiates at one lower corner of retentate member 422—specifically at retentate port 428—traverses across and up then down and across retentate member 422, ending in the other lower corner of retentate member 422 at a second retentate port 428. Also seen on retentate member 422 are energy directors 491, which circumscribe the region where a membrane or filter (not seen in this FIG. 4A) is seated, as well as interdigitate between areas of channel 402. Energy directors 491 in this embodiment mate with and serve to facilitate ultrasonic welding or bonding of retentate member 422 with permeate/filtrate member 420 via the energy director component 491 on permeate/filtrate member 420 (at right). Additionally, countersinks 423 can be seen, two on the bottom one at the top middle of retentate member 422. Countersinks 423 are used to couple and tangential flow assembly 410 to a reservoir assembly (not seen in this FIG. 4A but see FIG. 4B).

Permeate/filtrate member 420 is seen in the middle of FIG. 4A and comprises, in addition to energy director 491, through-holes for retentate ports 428 at each bottom corner (which mate with the through-holes for retentate ports 428 at the bottom corners of retentate member 422), as well as a tangential flow channel 402 and two permeate/filtrate ports 426 positioned at the top and center of permeate member 420. The tangential flow channel 402 structure in this embodiment has a serpentine configuration and an undulating geometry, although other geometries may be used. Permeate member 420 also comprises countersinks 423, coincident with the countersinks 423 on retentate member 420.

On the left of FIG. 4A is a tangential flow assembly 410 comprising the retentate member 422 and permeate member 420 seen in this FIG. 4A. In this view, retentate member 422 is “on top” of the view, a membrane (not seen in this view of the assembly) would be adjacent and under retentate member 422 and permeate member 420 (also not seen in this view of the assembly) is adjacent to and beneath the membrane. Again countersinks 423 are seen, where the countersinks in the retentate member 422 and the permeate member 420 are coincident and configured to mate with threads or mating elements for the countersinks disposed on a reservoir assembly (not seen in FIG. 4A but see FIG. 4B).

A membrane or filter is disposed between the retentate and permeate members, where fluids can flow through the membrane but cells cannot and are thus retained in the flow channel disposed in the retentate member. Filters or membranes appropriate for use in the TFF device/module are those that are solvent resistant, are contamination free during filtration, and are able to retain the types and sizes of cells of interest. For example, in order to retain small cell types such as bacterial cells, pore sizes can be as low as 0.2 μm, however for other cell types, the pore sizes can be as high as 20 μm. Indeed, the pore sizes useful in the TFF device/module include filters with sizes from 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable non-reactive material including cellulose mixed ester (cellulose nitrate and acetate) (CME), polycarbonate (PC), polyvinylidene fluoride (PVDF), polyethersulfone (PES), polytetrafluoroethylene (PTFE), nylon, glass fiber, or metal substrates as in the case of laser or electrochemical etching.

The length of the channel structure 402 may vary depending on the volume of the cell culture to be grown and the optical density of the cell culture to be concentrated. The length of the channel structure typically is from 60 mm to 300 mm, or from 70 mm to 200 mm, or from 80 mm to 100 mm. The cross-section configuration of the flow channel 402 may be round, elliptical, oval, square, rectangular, trapezoidal, or irregular. If square, rectangular, or another shape with generally straight sides, the cross section may be from about 10 μm to 1000 μm wide, or from 200 μm to 800 μm wide, or from 300 μm to 700 μm wide, or from 400 μm to 600 μm wide; and from about 10 μm to 1000 μm high, or from 200 μm to 800 μm high, or from 300 μm to 700 μm high, or from 400 μm to 600 μm high. If the cross section of the flow channel 102 is generally round, oval or elliptical, the radius of the channel may be from about 50 μm to 1000 μm in hydraulic radius, or from 5 μm to 800 μm in hydraulic radius, or from 200 μm to 700 μm in hydraulic radius, or from 300 μm to 600 μm wide in hydraulic radius, or from about 200 to 500 μm in hydraulic radius. Moreover, the volume of the channel in the retentate 422 and permeate 420 members may be different depending on the depth of the channel in each member.

FIG. 4B shows front perspective (right) and rear perspective (left) views of a reservoir assembly 450 configured to be used with the tangential flow assembly 410 seen in FIG. 4A. Seen in the front perspective view (e.g., “front” being the side of reservoir assembly 450 that is coupled to the tangential flow assembly 410 seen in FIG. 4A) are retentate reservoirs 452 on either side of permeate reservoir 454. Also seen are permeate ports 426, retentate ports 428, and three threads or mating elements 425 for countersinks 423 (countersinks 423 not seen in this FIG. 4B). Threads or mating elements 425 for countersinks 423 are configured to mate or couple the tangential flow assembly 410 (seen in FIG. 4A) to reservoir assembly 450. Alternatively or in addition, fasteners, sonic welding or heat stakes may be used to mate or couple the tangential flow assembly 410 to reservoir assembly 450. In addition is seen gasket 445 covering the top of reservoir assembly 450. Gasket 445 is described in detail in relation to FIG. 4E. At left in FIG. 4B is a rear perspective view of reservoir assembly 1250, where “rear” is the side of reservoir assembly 450 that is not coupled to the tangential flow assembly. Seen are retentate reservoirs 452, permeate reservoir 454, and gasket 445.

The TFF device may be fabricated from any robust material in which channels (and channel branches) may be milled including stainless steel, silicon, glass, aluminum, or plastics including cyclic-olefin copolymer (COC), cyclo-olefin polymer (COP), polystyrene, polyvinyl chloride, polyethylene, polyamide, polyethylene, polypropylene, acrylonitrile butadiene, polycarbonate, polyetheretheketone (PEEK), poly(methyl methylacrylate) (PMMA), polysulfone, and polyurethane, and co-polymers of these and other polymers. If the TFF device/module is disposable, preferably it is made of plastic. In some embodiments, the material used to fabricate the TFF device/module is thermally-conductive so that the cell culture may be heated or cooled to a desired temperature. In certain embodiments, the TFF device is formed by precision mechanical machining, laser machining, electro discharge machining (for metal devices); wet or dry etching (for silicon devices); dry or wet etching, powder or sandblasting, photostructuring (for glass devices); or thermoforming, injection molding, hot embossing, or laser machining (for plastic devices) using the materials mentioned above that are amenable to this mass production techniques.

FIG. 4C depicts a top-down view of the reservoir assemblies 450 shown in FIG. 4B. FIG. 4D depicts a cover 444 for reservoir assembly 450 shown in FIGS. 4B and 4E depicts a gasket 445 that in operation is disposed on cover 444 of reservoir assemblies 450 shown in FIG. 4B. FIG. 4C is a top-down view of reservoir assembly 450, showing the tops of the two retentate reservoirs 452, one on either side of permeate reservoir 454. Also seen are grooves 432 that will mate with a pneumatic port (not shown), and fluid channels 434 that reside at the bottom of retentate reservoirs 452, which fluidically couple the retentate reservoirs 452 with the retentate ports 428 (not shown), via the through-holes for the retentate ports in permeate member 420 and membrane 424 (also not shown). FIG. 4D depicts a cover 444 that is configured to be disposed upon the top of reservoir assembly 450. Cover 444 has round cut-outs at the top of retentate reservoirs 452 and permeate/filtrate reservoir 454. Again at the bottom of retentate reservoirs 452 fluid channels 434 can be seen, where fluid channels 434 fluidically couple retentate reservoirs 452 with the retentate ports 428 (not shown). Also shown are three pneumatic ports 430 for each retentate reservoir 452 and permeate/filtrate reservoir 454. FIG. 4E depicts a gasket 445 that is configures to be disposed upon the cover 444 of reservoir assembly 450. Seen are three fluid transfer ports 442 for each retentate reservoir 452 and for permeate/filtrate reservoir 454. Again, three pneumatic ports 430, for each retentate reservoir 452 and for permeate/filtrate reservoir 454, are shown.

The overall work flow for cell growth comprises loading a cell culture to be grown into a first retentate reservoir, optionally bubbling air or an appropriate gas through the cell culture, passing or flowing the cell culture through the first retentate port then tangentially through the TFF channel structure while collecting medium or buffer through one or both of the permeate ports 406, collecting the cell culture through a second retentate port 404 into a second retentate reservoir, optionally adding additional or different medium to the cell culture and optionally bubbling air or gas through the cell culture, then repeating the process, all while measuring, e.g., the optical density of the cell culture in the retentate reservoirs continuously or at desired intervals. Measurements of optical densities (OD) at programmed time intervals are accomplished using a 600 nm Light Emitting Diode (LED) that has been columnated through an optic into the retentate reservoir(s) containing the growing cells. The light continues through a collection optic to the detection system which consists of a (digital) gain-controlled silicone photodiode. Generally, optical density is shown as the absolute value of the logarithm with base 10 of the power transmission factors of an optical attenuator: OD=−log 10 (Power out/Power in). Since OD is the measure of optical attenuation—that is, the sum of absorption, scattering, and reflection—the TFF device OD measurement records the overall power transmission, so as the cells grow and become denser in population, the OD (the loss of signal) increases. The OD system is pre-calibrated against OD standards with these values stored in an on-board memory accessible by the measurement program.

In the channel structure, the membrane bifurcating the flow channels retains the cells on one side of the membrane (the retentate side 422) and allows unwanted medium or buffer to flow across the membrane into a filtrate or permeate side (e.g., permeate member 420) of the device. Bubbling air or other appropriate gas through the cell culture both aerates and mixes the culture to enhance cell growth. During the process, medium that is removed during the flow through the channel structure is removed through the permeate/filtrate ports 406. Alternatively, cells can be grown in one reservoir with bubbling or agitation without passing the cells through the TFF channel from one reservoir to the other.

The overall work flow for cell concentration using the TFF device/module involves flowing a cell culture or cell sample tangentially through the channel structure. As with the cell growth process, the membrane bifurcating the flow channels retains the cells on one side of the membrane and allows unwanted medium or buffer to flow across the membrane into a permeate/filtrate side (e.g., permeate member 420) of the device. In this process, a fixed volume of cells in medium or buffer is driven through the device until the cell sample is collected into one of the retentate ports 404, and the medium/buffer that has passed through the membrane is collected through one or both of the permeate/filtrate ports 406. All types of prokaryotic and eukaryotic cells—both adherent and non-adherent cells—can be grown in the TFF device. Adherent cells may be grown on beads or other cell scaffolds suspended in medium that flow through the TFF device.

The medium or buffer used to suspend the cells in the cell concentration device/module may be any suitable medium or buffer for the type of cells being transformed or transfected, such as LB, SOC, TPD, YPG, YPAD, MEM, DMEM, IMDM, RPMI, Hanks', PBS and Ringer's solution, where the media may be provided in a reagent cartridge as part of a kit. For culture of adherent cells, cells may be disposed on beads, microcarriers, or other type of scaffold suspended in medium. Most normal mammalian tissue-derived cells— except those derived from the hematopoietic system—are anchorage dependent and need a surface or cell culture support for normal proliferation. In the rotating growth vial described herein, microcarrier technology is leveraged. Microcarriers of particular use typically have a diameter of 100-300 μm and have a density slightly greater than that of the culture medium (thus facilitating an easy separation of cells and medium for, e.g., medium exchange) yet the density must also be sufficiently low to allow complete suspension of the carriers at a minimum stirring rate in order to avoid hydrodynamic damage to the cells. Many different types of microcarriers are available, and different microcarriers are optimized for different types of cells. There are positively charged carriers, such as Cytodex 1 (dextran-based, GE Healthcare), DE-52 (cellulose-based, Sigma-Aldrich Labware), DE-53 (cellulose-based, Sigma-Aldrich Labware), and HLX 11-170 (polystyrene-based); collagen- or ECM- (extracellular matrix) coated carriers, such as Cytodex 3 (dextran-based, GE Healthcare) or HyQ-sphere Pro-F 102-4 (polystyrene-based, Thermo Scientific); non-charged carriers, like HyQ-sphere P 102-4 (Thermo Scientific); or macroporous carriers based on gelatin (Cultisphere, Percell Biolytica) or cellulose (Cytopore, GE Healthcare).

In both the cell growth and concentration processes, passing the cell sample through the TFF device and collecting the cells in one of the retentate ports 404 while collecting the medium in one of the permeate/filtrate ports 406 is considered “one pass” of the cell sample. The transfer between retentate reservoirs “flips” the culture. The retentate and permeatee ports collecting the cells and medium, respectively, for a given pass reside on the same end of TFF device/module with fluidic connections arranged so that there are two distinct flow layers for the retentate and permeate/filtrate sides, but if the retentate port 404 resides on the retentate member of device/module (that is, the cells are driven through the channel above the membrane and the filtrate (medium) passes to the portion of the channel below the membrane), the permeate/filtrate port 406 will reside on the permeate member of device/module and vice versa (that is, if the cell sample is driven through the channel below the membrane, the filtrate (medium) passes to the portion of the channel above the membrane). Due to the high pressures used to transfer the cell culture and fluids through the flow channel of the TFF device, the effect of gravity is negligible.

At the conclusion of a “pass” in either of the growth and concentration processes, the cell sample is collected by passing through the retentate port 404 and into the retentate reservoir (not shown). To initiate another “pass”, the cell sample is passed again through the TFF device, this time in a flow direction that is reversed from the first pass. The cell sample is collected by passing through the retentate port 404 and into retentate reservoir (not shown) on the opposite end of the device/module from the retentate port 404 that was used to collect cells during the first pass. Likewise, the medium/buffer that passes through the membrane on the second pass is collected through the permeate port 406 on the opposite end of the device/module from the permeate port 406 that was used to collect the filtrate during the first pass, or through both ports. This alternating process of passing the retentate (the concentrated cell sample) through the device/module is repeated until the cells have been grown to a desired optical density, and/or concentrated to a desired volume, and both permeate ports (i.e., if there are more than one) can be open during the passes to reduce operating time. In addition, buffer exchange may be effected by adding a desired buffer (or fresh medium) to the cell sample in the retentate reservoir, before initiating another “pass”, and repeating this process until the old medium or buffer is diluted and filtered out and the cells reside in fresh medium or buffer. Note that buffer exchange and cell growth may (and typically do) take place simultaneously, and buffer exchange and cell concentration may (and typically do) take place simultaneously. For further information and alternative embodiments on TFFs see, e.g., U.S. Ser. No. 62/728,365, filed 7 Sep. 2018; 62/857,599, filed 5 Jun. 2019; and 62/867,415, filed 27 Jun. 2019.

### The Cell Transformation Module

FIG. 5A depicts an exemplary combination reagent cartridge and electroporation device 500 (“cartridge”) that may be used in an automated multi-module cell processing instrument along with the TFF module. In addition, in certain embodiments the material used to fabricate the cartridge is thermally-conductive, as in certain embodiments the cartridge 500 contacts a thermal device (not shown), such as a Peltier device or thermoelectric cooler, that heats or cools reagents in the reagent reservoirs or reservoirs 504. Reagent reservoirs or reservoirs 504 may be reservoirs into which individual tubes of reagents are inserted as shown in FIG. 5A, or the reagent reservoirs may hold the reagents without inserted tubes. Additionally, the reservoirs in a reagent cartridge may be configured for any combination of tubes, co-joined tubes, and direct-fill of reagents.

In one embodiment, the reagent reservoirs or reservoirs 504 of reagent cartridge 500 are configured to hold various size tubes, including, e.g., 250 ml tubes, 25 ml tubes, 10 ml tubes, 5 ml tubes, and Eppendorf or microcentrifuge tubes. In yet another embodiment, all reservoirs may be configured to hold the same size tube, e.g., 5 ml tubes, and reservoir inserts may be used to accommodate smaller tubes in the reagent reservoir. In yet another embodiment—particularly in an embodiment where the reagent cartridge is disposable—the reagent reservoirs hold reagents without inserted tubes. In this disposable embodiment, the reagent cartridge may be part of a kit, where the reagent cartridge is pre-filled with reagents and the receptacles or reservoirs sealed with, e.g., foil, heat seal acrylic or the like and presented to a consumer where the reagent cartridge can then be used in an automated multi-module cell processing instrument. As one of ordinary skill in the art will appreciate given the present disclosure, the reagents contained in the reagent cartridge will vary depending on work flow; that is, the reagents will vary depending on the processes to which the cells are subjected in the automated multi-module cell processing instrument, e.g., protein production, cell transformation and culture, cell editing, etc.

Reagents such as cell samples, enzymes, buffers, nucleic acid vectors, expression cassettes, proteins or peptides, reaction components (such as, e.g., MgCl₂, dNTPs, nucleic acid assembly reagents, gap repair reagents, and the like), wash solutions, ethanol, and magnetic beads for nucleic acid purification and isolation, etc. may be positioned in the reagent cartridge at a known position. In some embodiments of cartridge 500, the cartridge comprises a script (not shown) readable by a processor (not shown) for dispensing the reagents. Also, the cartridge 500 as one component in an automated multi-module cell processing instrument may comprise a script specifying two, three, four, five, ten or more processes to be performed by the automated multi-module cell processing instrument. In certain embodiments, the reagent cartridge is disposable and is pre-packaged with reagents tailored to performing specific cell processing protocols, e.g., genome editing or protein production. Because the reagent cartridge contents vary while components/modules of the automated multi-module cell processing instrument or system may not, the script associated with a particular reagent cartridge matches the reagents used and cell processes performed. Thus, e.g., reagent cartridges may be pre-packaged with reagents for genome editing and a script that specifies the process steps for performing genome editing in an automated multi-module cell processing instrument, or, e.g., reagents for protein expression and a script that specifies the process steps for performing protein expression in an automated multi-module cell processing instrument.

For example, the reagent cartridge may comprise a script to pipette competent cells from a reservoir, transfer the cells to a transformation module, pipette a nucleic acid solution comprising a vector with expression cassette from another reservoir in the reagent cartridge, transfer the nucleic acid solution to the transformation module, initiate the transformation process for a specified time, then move the transformed cells to yet another reservoir in the reagent cassette or to another module such as a cell growth module in the automated multi-module cell processing instrument. In another example, the reagent cartridge may comprise a script to transfer a nucleic acid solution comprising a vector from a reservoir in the reagent cassette, nucleic acid solution comprising editing oligonucleotide cassettes in a reservoir in the reagent cassette, and a nucleic acid assembly mix from another reservoir to the nucleic acid assembly/desalting module, if present. The script may also specify process steps performed by other modules in the automated multi-module cell processing instrument. For example, the script may specify that the nucleic acid assembly/desalting reservoir be heated to 50° C. for 30 min to generate an assembled product; and desalting and resuspension of the assembled product via magnetic bead-based nucleic acid purification involving a series of pipette transfers and mixing of magnetic beads, ethanol wash, and buffer.

As described in relation to FIGS. 5B and 5C below, the exemplary reagent cartridges for use in the automated multi-module cell processing instruments may include one or more electroporation devices, preferably flow-through electroporation (FTEP) devices. In yet other embodiments, the reagent cartridge is separate from the transformation module. Electroporation is a widely-used method for permeabilization of cell membranes that works by temporarily generating pores in the cell membranes with electrical stimulation. Applications of electroporation include the delivery of DNA, RNA, siRNA, peptides, proteins, antibodies, drugs or other substances to a variety of cells such as mammalian cells (including human cells), plant cells, archea, yeasts, other eukaryotic cells, bacteria, and other cell types. Electrical stimulation may also be used for cell fusion in the production of hybridomas or other fused cells. During a typical electroporation procedure, cells are suspended in a buffer or medium that is favorable for cell survival. For bacterial cell electroporation, low conductance mediums, such as water, glycerol solutions and the like, are often used to reduce the heat production by transient high current. In traditional electroporation devices, the cells and material to be electroporated into the cells (collectively “the cell sample”) are placed in a cuvette embedded with two flat electrodes for electrical discharge. For example, Bio-Rad (Hercules, Calif.) makes the GENE PULSER XCELL™ line of products to electroporate cells in cuvettes. Traditionally, electroporation requires high field strength; however, the flow-through electroporation devices included in the reagent cartridges achieve high efficiency cell electroporation with low toxicity. The reagent cartridges of the disclosure allow for particularly easy integration with robotic liquid handling instrumentation that is typically used in automated instruments and systems such as air displacement pipettors. Such automated instrumentation includes, but is not limited to, off-the-shelf automated liquid handling systems from Tecan (Mannedorf, Switzerland), Hamilton (Reno, Nev.), Beckman Coulter (Fort Collins, Colo.), etc.

FIGS. 5B and 5C are top perspective and bottom perspective views, respectively, of an exemplary FTEP device 550 that may be part of (e.g., a component in) reagent cartridge 500 in FIG. 5A or may be a stand-alone module; that is, not a part of a reagent cartridge or other module. FIG. 5B depicts an FTEP device 550. The FTEP device 550 has wells that define cell sample inlets 552 and cell sample outlets 554. FIG. 5C is a bottom perspective view of the FTEP device 550 of FIG. 5B. An inlet well 552 and an outlet well 554 can be seen in this view. Also seen in FIG. 5C are the bottom of an inlet 562 corresponding to well 552, the bottom of an outlet 564 corresponding to the outlet well 554, the bottom of a defined flow channel 566 and the bottom of two electrodes 568 on either side of flow channel 566. The FTEP devices may comprise push-pull pneumatic means to allow multi-pass electroporation procedures; that is, cells to electroporated may be “pulled” from the inlet toward the outlet for one pass of electroporation, then be “pushed” from the outlet end of the FTEP device toward the inlet end to pass between the electrodes again for another pass of electroporation. Further, this process may be repeated one to many times. For additional information regarding FTEP devices, see, e.g., U.S. Ser. No. 16/147,120, filed 28 Sep. 2018; Ser. No. 16/147,353, filed 28 Sep. 2018; Ser. No. 16/426,310, filed 30 May 2019; and Ser. No. 16/147,871, filed 30 Sep. 2018; and U.S. Pat. No. 10,323,258, issued 18 Jun. 2019. Further, other embodiments of the reagent cartridge may provide or accommodate electroporation devices that are not configured as FTEP devices, such as those described in U.S. Ser. No. 16/109,156, filed 22 Aug. 2018. For reagent cartridges useful in the present automated multi-module cell processing instruments, see, e.g., U.S. Pat. No. 10,376,889, issued 13 Aug. 2019; and U.S. Ser. No. 16,451,601, filed 25 Jun. 2019.

Additional details of the FTEP devices are illustrated in FIGS. 5D-5F. Note that in the FTEP devices in FIGS. 5D-5F the electrodes are placed such that a first electrode is placed between an inlet and a narrowed region of the flow channel, and the second electrode is placed between the narrowed region of the flow channel and an outlet. FIG. 5D shows a top planar view of an FTEP device 550 having an inlet 552 for introducing a fluid containing cells and exogenous material into FTEP device 550 and an outlet 554 for removing the transformed cells from the FTEP following electroporation. The electrodes 568 are introduced through channels (not shown) in the device. FIG. 5E shows a cutaway view from the top of the FTEP device 550, with the inlet 552, outlet 554, and electrodes 568 positioned with respect to a flow channel 566. FIG. 5F shows a side cutaway view of FTEP device 550 with the inlet 552 and inlet channel 572, and outlet 554 and outlet channel 574. The electrodes 568 are positioned in electrode channels 576 so that they are in fluid communication with the flow channel 566, but not directly in the path of the cells traveling through the flow channel 566. Note that the first electrode is placed between the inlet and the narrowed region of the flow channel, and the second electrode is placed between the narrowed region of the flow channel and the outlet. The electrodes 568 in this aspect of the device are positioned in the electrode channels 576 which are generally perpendicular to the flow channel 566 such that the fluid containing the cells and exogenous material flows from the inlet channel 572 through the flow channel 566 to the outlet channel 574, and in the process fluid flows into the electrode channels 376 to be in contact with the electrodes 568. In this aspect, the inlet channel, outlet channel and electrode channels all originate from the same planar side of the device. In certain aspects, however, the electrodes may be introduced from a different planar side of the FTEP device than the inlet and outlet channels.

In the FTEP devices of the disclosure, the toxicity level of the transformation results in greater than 30% viable cells after electroporation, preferably greater than 35%, 40%, 45%, 50%, 55%, 60%, 70%, 75%, 80%, 85%, 90%, 95% or even 99% viable cells following transformation, depending on the cell type and the nucleic acids being introduced into the cells.

The housing of the FTEP device can be made from many materials depending on whether the FTEP device is to be reused, autoclaved, or is disposable, including stainless steel, silicon, glass, resin, polyvinyl chloride, polyethylene, polyamide, polystyrene, polyethylene, polypropylene, acrylonitrile butadiene, polycarbonate, polyetheretheketone (PEEK), polysulfone and polyurethane, co-polymers of these and other polymers. Similarly, the walls of the channels in the device can be made of any suitable material including silicone, resin, glass, glass fiber, polyvinyl chloride, polyethylene, polyamide, polyethylene, polypropylene, acrylonitrile butadiene, polycarbonate, polyetheretheketone (PEEK), polysulfone and polyurethane, co-polymers of these and other polymers. Preferred materials include crystal styrene, cyclo-olefin polymer (COP) and cyclic olephin co-polymers (COC), which allow the device to be formed entirely by injection molding in one piece with the exception of the electrodes and, e.g., a bottom sealing film if present.

The FTEP devices described herein (or portions of the FTEP devices) can be created or fabricated via various techniques, e.g., as entire devices or by creation of structural layers that are fused or otherwise coupled. For example, for metal FTEP devices, fabrication may include precision mechanical machining or laser machining; for silicon FTEP devices, fabrication may include dry or wet etching; for glass FTEP devices, fabrication may include dry or wet etching, powderblasting, sandblasting, or photostructuring; and for plastic FTEP devices fabrication may include thermoforming, injection molding, hot embossing, or laser machining. The components of the FTEP devices may be manufactured separately and then assembled, or certain components of the FTEP devices (or even the entire FTEP device except for the electrodes) may be manufactured (e.g., using 3D printing) or molded (e.g., using injection molding) as a single entity, with other components added after molding. For example, housing and channels may be manufactured or molded as a single entity, with the electrodes later added to form the FTEP unit. Alternatively, the FTEP device may also be formed in two or more parallel layers, e.g., a layer with the horizontal channel and filter, a layer with the vertical channels, and a layer with the inlet and outlet ports, which are manufactured and/or molded individually and assembled following manufacture.

In specific aspects, the FTEP device can be manufactured using a circuit board as a base, with the electrodes, filter and/or the flow channel formed in the desired configuration on the circuit board, and the remaining housing of the device containing, e.g., the one or more inlet and outlet channels and/or the flow channel formed as a separate layer that is then sealed onto the circuit board. The sealing of the top of the housing onto the circuit board provides the desired configuration of the different elements of the FTEP devices of the disclosure. Also, two to many FTEP devices may be manufactured on a single substrate, then separated from one another thereafter or used in parallel. In certain embodiments, the FTEP devices are reusable and, in some embodiments, the FTEP devices are disposable. In additional embodiments, the FTEP devices may be autoclavable.

The electrodes 408 can be formed from any suitable metal, such as copper, stainless steel, titanium, aluminum, brass, silver, rhodium, gold or platinum, or graphite. One preferred electrode material is alloy 303 (UNS330300) austenitic stainless steel. An applied electric field can destroy electrodes made from of metals like aluminum. If a multiple-use (i.e., non-disposable) flow-through FTEP device is desired-as opposed to a disposable, one-use flow-through FTEP device—the electrode plates can be coated with metals resistant to electrochemical corrosion. Conductive coatings like noble metals, e.g., gold, can be used to protect the electrode plates.

As mentioned, the FTEP devices may comprise push-pull pneumatic means to allow multi-pass electroporation procedures; that is, cells to electroporated may be “pulled” from the inlet toward the outlet for one pass of electroporation, then be “pushed” from the outlet end of the flow-through FTEP device toward the inlet end to pass between the electrodes again for another pass of electroporation. This process may be repeated one to many times.

Depending on the type of cells to be electroporated (e.g., bacterial, yeast, mammalian) and the configuration of the electrodes, the distance between the electrodes in the flow channel can vary widely. For example, where the flow channel decreases in width, the flow channel may narrow to between 10 μm and 5 mm, or between 25 μm and 3 mm, or between 50 μm and 2 mm, or between 75 μm and 1 mm. The distance between the electrodes in the flow channel may be between 1 mm and 10 mm, or between 2 mm and 8 mm, or between 3 mm and 7 mm, or between 4 mm and 6 mm. The overall size of the FTEP device may be from 3 cm to 15 cm in length, or 4 cm to 12 cm in length, or 4.5 cm to 10 cm in length. The overall width of the FTEP device may be from 0.5 cm to 5 cm, or from 0.75 cm to 3 cm, or from 1 cm to 2.5 cm, or from 1 cm to 1.5 cm.

The region of the flow channel that is narrowed is wide enough so that at least two cells can fit in the narrowed portion side-by-side. For example, a typical bacterial cell is 1 μm in diameter; thus, the narrowed portion of the flow channel of the FTEP device used to transform such bacterial cells will be at least 2 μm wide. In another example, if a mammalian cell is approximately 50 μm in diameter, the narrowed portion of the flow channel of the FTEP device used to transform such mammalian cells will be at least 100 μm wide. That is, the narrowed portion of the FTEP device will not physically contort or “squeeze” the cells being transformed.

In embodiments of the FTEP device where reservoirs are used to introduce cells and exogenous material into the FTEP device, the reservoirs range in volume from 100 μL to 10 mL, or from 500 μL to 75 mL, or from 1 mL to 5 mL. The flow rate in the FTEP ranges from 0.1 mL to 5 mL per minute, or from 0.5 mL to 3 mL per minute, or from 1.0 mL to 2.5 mL per minute. The pressure in the FTEP device ranges from 1-30 psi, or from 2-10 psi, or from 3-5 psi.

To avoid different field intensities between the electrodes, the electrodes should be arranged in parallel. Furthermore, the surface of the electrodes should be as smooth as possible without pin holes or peaks. Electrodes having a roughness Rz of 1 to 10 μm are preferred. In another embodiment of the invention, the flow-through electroporation device comprises at least one additional electrode which applies a ground potential to the FTEP device. Flow-through electroporation devices (either as a stand-alone instrument or as a module in an automated multi-module system) are described in, e.g., U.S. Ser. No. 16/147,120, filed 28 Sep. 2018; Ser. No. 16/147,353, filed 28 Sep. 2018; Ser. No. 16/147,865, filed 30 Sep. 2018; and Ser. No. 16/426,310, filed 30 May 2019; and U.S. Pat. No. 10,323,258, issued 18 Jun. 2019.

### Cell Singulation and Enrichment Device

FIG. 6A depicts a solid wall device 6050 and a workflow for singulating cells in microwells in the solid wall device. At the top left of the figure (i), there is depicted solid wall device 6050 with microwells 6052. A section 6054 of substrate 6050 is shown at (ii), also depicting microwells 6052. At (iii), a side cross-section of solid wall device 6050 is shown, and microwells 6052 have been loaded, where, in this embodiment, Poisson or substantial Poisson loading has taken place; that is, each microwell has one or no cells, and the likelihood that any one microwell has more than one cell is low. At (iv), workflow 6040 is illustrated where substrate 6050 having microwells 6052 shows microwells 6056 with one cell per microwell, microwells 6057 with no cells in the microwells, and one microwell 6060 with two cells in the microwell. In step 6051, the cells in the microwells are allowed to double approximately 2-150 times to form clonal colonies (v), then editing is allowed to occur 6053.

After editing 6053, many cells in the colonies of cells that have been edited die as a result of the double-strand cuts caused by active editing and there is a lag in growth for the edited cells that do survive but must repair and recover following editing (microwells 6058), where cells that do not undergo editing thrive (microwells 6059) (vi). All cells are allowed to continue grow to establish colonies and normalize, where the colonies of edited cells in microwells 6058 catch up in size and/or cell number with the cells in microwells 6059 that do not undergo editing (vii). Once the cell colonies are normalized, either pooling 6060 of all cells in the microwells can take place, in which case the cells are enriched for edited cells by eliminating the bias from non-editing cells and fitness effects from editing; alternatively, colony growth in the microwells is monitored after editing, and slow growing colonies (e.g., the cells in microwells 6058) are identified and selected 6061 (e.g., “cherry picked”) resulting in even greater enrichment of edited cells.

In growing the cells, the medium used will depend, of course, on the type of cells being edited—e.g., bacterial, yeast or mammalian. For example, medium for yeast cell growth includes LB, SOC, TPD, YPG, YPAD, MEM and DMEM.

FIG. 6B depicts a solid wall device 6050 and a workflow for substantially singulating cells in microwells in a solid wall device. At the top left of the figure (i), there is depicted solid wall device 350 with microwells 6052. A section 6054 of substrate 6050 is shown at (ii), also depicting microwells 6052. At (iii), a side cross-section of solid wall device 6050 is shown, and microwells 6052 have been loaded, where, in this embodiment, substantial Poisson loading has taken place; that is, some microwells 6057 have no cells, and some microwells 6076, 6078 have a few cells. In FIG. 6B, cells with active gRNAs are shown as solid circles, and cells with inactive gRNAs are shown as open circles. At (iv), workflow 6070 is illustrated where substrate 6050 having microwells 6052 shows three microwells 6076 with several cells all with active gRNAs, microwell 6057 with no cells, and two microwells 6078 with some cells having active gRNAs and some cells having inactive gRNAs. In step 6071, the cells in the microwells are allowed to double approximately 2-150 times to form clonal colonies (v), then editing takes place 6073.

After editing 6073, many cells in the colonies of cells that have been edited die as a result of the double-strand cuts caused by active editing and there is a lag in growth for the edited cells that do survive but must repair and recover following editing (microwells 6076), where cells that do not undergo editing thrive (microwells 6078) (vi). Thus, in microwells 6076 where only cells with active gRNAs reside (cells depicted by solid circles), most cells die off; however, in microwells 6078 containing cells with inactive gRNAs (cells depicted by open circles), cells continue to grow and are not impacted by active editing. The cells in each microwell (6076 and 6078) are allowed to grow to continue to establish colonies and normalize, where the colonies of edited cells in microwells 6076 catch up in size and/or cell number with the unedited cells in microwells 6078 that do not undergo editing (vii). Note that in this workflow 6070, the colonies of cells in the microwells are not clonal; that is, not all cells in a well arise from a single cell. Instead, the cell colonies in the well may be mixed colonies, arising in many wells from two to several different cells. Once the cell colonies are normalized, either pooling 6090 of all cells in the microwells can take place, in which case the cells are enriched for edited cells by eliminating the bias from non-editing cells and fitness effects from editing; alternatively, colony growth in the microwells is monitored after editing, and slow growing colonies (e.g., the cells in microwells 6076) are identified and selected 6091 (e.g., “cherry picked”) resulting in even greater enrichment of edited cells.

A module useful for performing the methods depicted in FIGS. 6A and 6B is a solid wall isolation, incubation, and normalization (SWIIN) module. FIG. 6C depicts an embodiment of a SWIIN module 650 from an exploded top perspective view. In SWIIN module 650 the retentate member is formed on the bottom of a top of a SWIIN module component and the permeate member is formed on the top of the bottom of a SWIIN module component.

The SWIIN module 650 in FIG. 6C comprises from the top down, a reservoir gasket or cover 658, a retentate member 604 (where a retentate flow channel cannot be seen in this FIG. 6C), a perforated member 601 swaged with a filter (filter not seen in FIG. 6C), a permeate member 608 comprising integrated reservoirs (permeate reservoirs 652 and retentate reservoirs 654), and two reservoir seals 662, which seal the bottom of permeate reservoirs 652 and retentate reservoirs 654. A permeate channel 660a can be seen disposed on the top of permeate member 608, defined by a raised portion 676 of serpentine channel 660a, and ultrasonic tabs 664 can be seen disposed on the top of permeate member 608 as well. The perforations that form the wells on perforated member 601 are not seen in this FIG. 6C; however, through-holes 666 to accommodate the ultrasonic tabs 664 are seen. In addition, supports 670 are disposed at either end of SWIIN module 650 to support SWIIN module 650 and to elevate permeate member 608 and retentate member 604 above reservoirs 652 and 654 to minimize bubbles or air entering the fluid path from the permeate reservoir to serpentine channel 660a or the fluid path from the retentate reservoir to serpentine channel 660b (neither fluid path is seen in this FIG. 6C).

In this FIG. 6C, it can be seen that the serpentine channel 660a that is disposed on the top of permeate member 608 traverses permeate member 608 for most of the length of permeate member 608 except for the portion of permeate member 608 that comprises permeate reservoirs 652 and retentate reservoirs 654 and for most of the width of permeate member 608. As used herein with respect to the distribution channels in the retentate member or permeate member, “most of the length” means about 95% of the length of the retentate member or permeate member, or about 90%, 85%, 80%, 75%, or 70% of the length of the retentate member or permeate member. As used herein with respect to the distribution channels in the retentate member or permeate member, “most of the width” means about 95% of the width of the retentate member or permeate member, or about 90%, 85%, 80%, 75%, or 70% of the width of the retentate member or permeate member.

In this embodiment of a SWIIN module, the perforated member includes through-holes to accommodate ultrasonic tabs disposed on the permeate member. Thus, in this embodiment the perforated member is fabricated from 316 stainless steel, and the perforations form the walls of microwells while a filter or membrane is used to form the bottom of the microwells. Typically, the perforations (microwells) are approximately 150 μm-200 μm in diameter, and the perforated member is approximately 125 μm deep, resulting in microwells having a volume of approximately 2.5 nl, with a total of approximately 200,000 microwells. The distance between the microwells is approximately 279 μm center-to-center. Though here the microwells have a volume of approximately 2.5 nl, the volume of the microwells may be from 1 to 25 nl, or preferably from 2 to 10 nl, and even more preferably from 2 to 4 nl. As for the filter or membrane, like the filter described previously, filters appropriate for use are solvent resistant, contamination free during filtration, and are able to retain the types and sizes of cells of interest. For example, in order to retain small cell types such as bacterial cells, pore sizes can be as low as 0.10 μm, however for other cell types (e.g., such as for mammalian cells), the pore sizes can be as high as 10.0 μm-20.0 μm or more. Indeed, the pore sizes useful in the cell concentration device/module include filters with sizes from 0.10 μm, 0.11 μm, 0.12 μm, 0.13 μm, 0.14 μm, 0.15 μm, 0.16 μm, 0.17 μm, 0.18 μm, 0.19 μm, 0.20 μm, 0.21 μm, 0.22 μm, 0.23 μm, 0.24 μm, 0.25 μm, 0.26 μm, 0.27 μm, 0.28 μm, 0.29 μm, 0.30 μm, 0.31 μm, 0.32 μm, 0.33 μm, 0.34 μm, 0.35 μm, 0.36 μm, 0.37 μm, 0.38 μm, 0.39 μm, 0.40 μm, 0.41 μm, 0.42 μm, 0.43 μm, 0.44 μm, 0.45 μm, 0.46 μm, 0.47 μm, 0.48 μm, 0.49 μm, 0.50 μm and larger. The filters may be fabricated from any suitable material including cellulose mixed ester (cellulose nitrate and acetate) (CME), polycarbonate (PC), polyvinylidene fluoride (PVDF), polyethersulfone (PES), polytetrafluoroethylene (PTFE), nylon, or glass fiber.

The cross-section configuration of the mated serpentine channel may be round, elliptical, oval, square, rectangular, trapezoidal, or irregular. If square, rectangular, or another shape with generally straight sides, the cross section may be from about 2 mm to 15 mm wide, or from 3 mm to 12 mm wide, or from 5 mm to 10 mm wide. If the cross section of the mated serpentine channel is generally round, oval or elliptical, the radius of the channel may be from about 3 mm to 20 mm in hydraulic radius, or from 5 mm to 15 mm in hydraulic radius, or from 8 mm to 12 mm in hydraulic radius.

Serpentine channels 660a and 660b can have approximately the same volume or a different volume. For example, each “side” or portion 660a, 660b of the serpentine channel may have a volume of, e.g., 2 mL, or serpentine channel 660a of permeate member 608 may have a volume of 2 mL, and the serpentine channel 660b of retentate member 604 may have a volume of, e.g., 3 mL. The volume of fluid in the serpentine channel may range from about 2 mL to about 80 mL, or about 4 mL to 60 mL, or from 5 mL to 40 mL, or from 6 mL to 20 mL (note these volumes apply to a SWIIN module comprising a, e.g., 50-500K perforation member). The volume of the reservoirs may range from 5 mL to 50 mL, or from 7 mL to 40 mL, or from 8 mL to 30 mL or from 10 mL to 20 mL, and the volumes of all reservoirs may be the same or the volumes of the reservoirs may differ (e.g., the volume of the permeate reservoirs is greater than that of the retentate reservoirs).

The serpentine channel portions 660a and 660b of the permeate member 608 and retentate member 604, respectively, are approximately 200 mm long, 130 mm wide, and 4 mm thick, though in other embodiments, the retentate and permeate members can be from 75 mm to 400 mm in length, or from 100 mm to 300 mm in length, or from 150 mm to 250 mm in length; from 50 mm to 250 mm in width, or from 75 mm to 200 mm in width, or from 100 mm to 150 mm in width; and from 2 mm to 15 mm in thickness, or from 4 mm to 10 mm in thickness, or from 5 mm to 8 mm in thickness. Embodiments the retentate (and permeate) members may be fabricated from PMMA (poly(methyl methacrylate) or other materials may be used, including polycarbonate, cyclic olefin co-polymer (COC), glass, polyvinyl chloride, polyethylene, polyamide, polypropylene, polysulfone, polyurethane, and co-polymers of these and other polymers. Preferably at least the retentate member is fabricated from a transparent material so that the cells can be visualized (see, e.g., FIG. 6F and the description thereof). For example, a video camera may be used to monitor cell growth by, e.g., density change measurements based on an image of an empty well, with phase contrast, or if, e.g., a chromogenic marker, such as a chromogenic protein, is used to add a distinguishable color to the cells. Chromogenic markers such as blitzen blue, dreidel teal, virginia violet, vixen purple, prancer purple, tinsel purple, maccabee purple, donner magenta, cupid pink, seraphina pink, scrooge orange, and leor orange (the Chromogenic Protein Paintbox, all available from ATUM (Newark, Calif.)) obviate the need to use fluorescence, although fluorescent cell markers, fluorescent proteins, and chemiluminescent cell markers may also be used.

Because the retentate member preferably is transparent, colony growth in the SWIIN module can be monitored by automated devices such as those sold by JoVE (ScanLag™ system, Cambridge, Mass.) (also see Levin-Reisman, et al., Nature Methods, 7:737-39 (2010)). Cell growth for, e.g., mammalian cells may be monitored by, e.g., the growth monitor sold by IncuCyte (Ann Arbor, Mich.) (see also, Choudhry, PLos One, 11(2):e0148469 (2016)). Further, automated colony pickers may be employed, such as those sold by, e.g., TECAN (Pickolo™ system, Mannedorf, Switzerland); Hudson Inc. (RapidPick™, Springfield, N.J.); Molecular Devices (QPix 400™ system, San Jose, Calif.); and Singer Instruments (PIXL™ system, Somerset, UK).

Due to the heating and cooling of the SWIIN module, condensation may accumulate on the retentate member which may interfere with accurate visualization of the growing cell colonies. Condensation of the SWIIN module 650 may be controlled by, e.g., moving heated air over the top of (e.g., retentate member) of the SWIIN module 650, or by applying a transparent heated lid over at least the serpentine channel portion 660b of the retentate member 604. See, e.g., FIG. 6F and the description thereof infra.

In SWIIN module 650 cells and medium—at a dilution appropriate for Poisson or substantial Poisson distribution of the cells in the microwells of the perforated member—are flowed into serpentine channel 660b from ports in retentate member 604, and the cells settle in the microwells while the medium passes through the filter into serpentine channel 660a in permeate member 608. The cells are retained in the microwells of perforated member 601 as the cells cannot travel through filter 603. Appropriate medium may be introduced into permeate member 608 through permeate ports 611. The medium flows upward through filter 603 to nourish the cells in the microwells (perforations) of perforated member 601. Additionally, buffer exchange can be effected by cycling medium through the retentate and permeate members. In operation, the cells are deposited into the microwells, are grown for an initial, e.g., 2-100 doublings, editing is induced by, e.g., raising the temperature of the SWIIN to 42° C. to induce a temperature inducible promoter or by removing growth medium from the permeate member and replacing the growth medium with a medium comprising a chemical component that induces an inducible promoter.

Once editing has taken place, the temperature of the SWIIN may be decreased, or the inducing medium may be removed and replaced with fresh medium lacking the chemical component thereby de-activating the inducible promoter. The cells then continue to grow in the SWIIN module 650 until the growth of the cell colonies in the microwells is normalized. For the normalization protocol, once the colonies are normalized, the colonies are flushed from the microwells by applying fluid or air pressure (or both) to the permeate member serpentine channel 660a and thus to filter 603 and pooled. Alternatively, if cherry picking is desired, the growth of the cell colonies in the microwells is monitored, and slow-growing colonies are directly selected; or, fast-growing colonies are eliminated.

FIG. 6D is a top perspective view of a SWIIN module with the retentate and perforated members in partial cross section. In this FIG. 6D, it can be seen that serpentine channel 660a is disposed on the top of permeate member 608 is defined by raised portions 676 and traverses permeate member 608 for most of the length and width of permeate member 608 except for the portion of permeate member 608 that comprises the permeate and retentate reservoirs (note only one retentate reservoir 652 can be seen). Moving from left to right, reservoir gasket 658 is disposed upon the integrated reservoir cover 678 (cover not seen in this FIG. 6D) of retentate member 604. Gasket 658 comprises reservoir access apertures 632a, 632b, 632c, and 632d, as well as pneumatic ports 633a, 633b, 633c and 633d. Also at the far left end is support 670. Disposed under permeate reservoir 652 can be seen one of two reservoir seals 662. In addition to the retentate member being in cross section, the perforated member 601 and filter 603 (filter 603 is not seen in this FIG. 6D) are in cross section. Note that there are a number of ultrasonic tabs 664 disposed at the right end of SWIIN module 650 and on raised portion 676 which defines the channel turns of serpentine channel 660a, including ultrasonic tabs 664 extending through through-holes 666 of perforated member 601. There is also a support 670 at the end distal reservoirs 652, 654 of permeate member 608.

FIG. 6E is a side perspective view of an assembled SWIIIN module 650, including, from right to left, reservoir gasket 658 disposed upon integrated reservoir cover 678 (not seen) of retentate member 604. Gasket 658 may be fabricated from rubber, silicone, nitrile rubber, polytetrafluoroethylene, a plastic polymer such as polychlorotrifluoroethylene, or other flexible, compressible material. Gasket 658 comprises reservoir access apertures 632a, 632b, 632c, and 632d, as well as pneumatic ports 633a, 633b, 633c and 633d. Also at the far-left end is support 670 of permeate member 608. In addition, permeate reservoir 652 can be seen, as well as one reservoir seal 662. At the far-right end is a second support 670.

Imaging of cell colonies growing in the wells of the SWIIN is desired in most implementations for, e.g., monitoring both cell growth and device performance and imaging is necessary for cherry-picking implementations. Real-time monitoring of cell growth in the SWIIN requires backlighting, retentate plate (top plate) condensation management and a system-level approach to temperature control, air flow, and thermal management. In some implementations, imaging employs a camera or CCD device with sufficient resolution to be able to image individual wells. For example, in some configurations a camera with a 9-pixel pitch is used (that is, there are 9 pixels center-to-center for each well). Processing the images may, in some implementations, utilize reading the images in grayscale, rating each pixel from low to high, where wells with no cells will be brightest (due to full or nearly-full light transmission from the backlight) and wells with cells will be dim (due to cells blocking light transmission from the backlight). After processing the images, thresholding is performed to determine which pixels will be called “bright” or “dim”, spot finding is performed to find bright pixels and arrange them into blocks, and then the spots are arranged on a hexagonal grid of pixels that correspond to the spots. Once arranged, the measure of intensity of each well is extracted, by, e.g., looking at one or more pixels in the middle of the spot, looking at several to many pixels at random or pre-set positions, or averaging X number of pixels in the spot. In addition, background intensity may be subtracted. Thresholding is again used to call each well positive (e.g., containing cells) or negative (e.g., no cells in the well). The imaging information may be used in several ways, including taking images at time points for monitoring cell growth. Monitoring cell growth can be used to, e.g., remove the “muffin tops” of fast-growing cells followed by removal of all cells or removal of cells in “rounds” as described above, or recover cells from specific wells (e.g., slow-growing cell colonies); alternatively, wells containing fast-growing cells can be identified and areas of UV light covering the fast-growing cell colonies can be projected (or rastered with shutters) onto the SWIIN to irradiate or inhibit growth of those cells. Imaging may also be used to assure proper fluid flow in the serpentine channel 660.

FIG. 6F depicts the embodiment of the SWIIN module in FIGS. 6A-6E further comprising a heat management system including a heater and a heated cover. The heater cover facilitates the condensation management that is required for imaging. Assembly 698 comprises a SWIIN module 650 seen lengthwise in cross section, where one permeate reservoir 652 is seen. Disposed immediately upon SWIIN module 650 is cover 694 and disposed immediately below SWIIN module 650 is backlight 680, which allows for imaging. Beneath and adjacent to the backlight and SWIIN module is insulation 682, which is disposed over a heatsink 684. In this FIG. 6F, the fins of the heatsink would be in-out of the page. In addition there is also axial fan 686 and heat sink 688, as well as two thermoelectric coolers 692, and a controller 690 to control the pneumatics, thermoelectric coolers, fan, solenoid valves, etc. The arrows denote cool air coming into the unit and hot air being removed from the unit. It should be noted that control of heating allows for growth of many different types of cells (prokaryotic and eukaryotic) as well as strains of cells that are, e.g., temperature sensitive, etc., and allows use of temperature-sensitive promoters. Temperature control allows for protocols to be adjusted to account for differences in transformation efficiency, cell growth and viability. For more details regarding solid wall isolation incubation and normalization devices see U.S. Ser. No. 16/399,988, file 30 Apr. 2019; Ser. No. 16/454,865, filed 26 Jun. 2019; and Ser. No. 16/540,606, filed 14 Aug. 2019. For alternative isolation, incubation and normalization modules, wee U.S. Ser. No. 16/536,049, filed 8 Aug. 2019.

### Use of the Automated Multi-Module Cell Processing Instrument

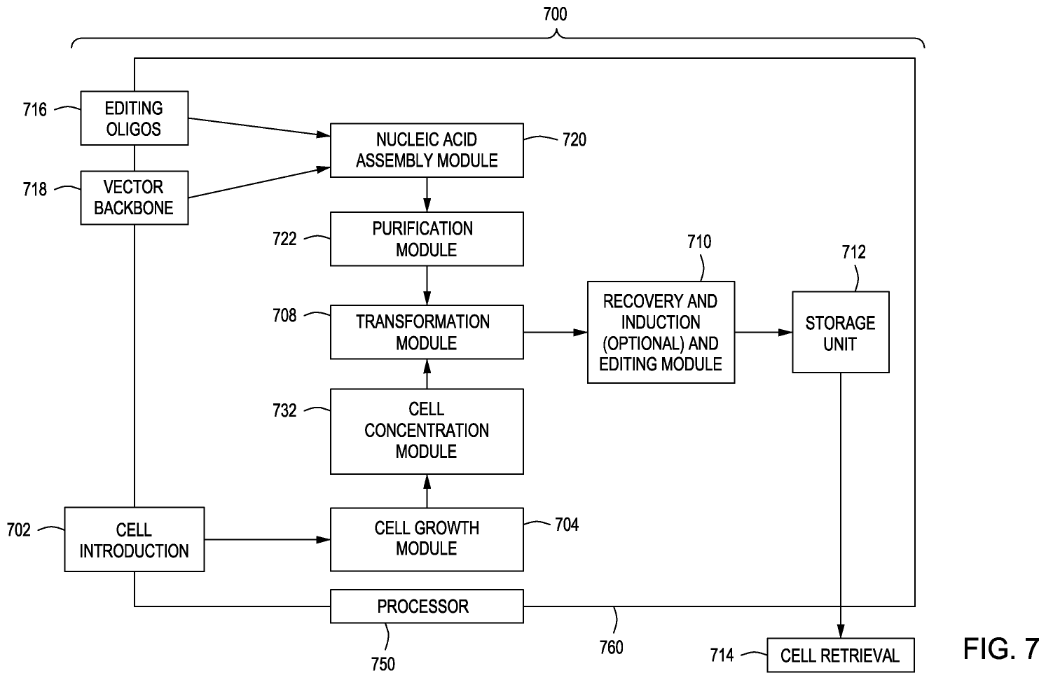

One embodiment of an automated multi-module cell processing instrument is shown in FIG. 7, where this embodiment is drawn to nucleic acid-guided nuclease editing. The cell processing instrument 700 may include a housing 760, a reservoir of cells in, e.g., the reagent cartridge to be transformed or transfected 702, and a cell growth module 704, separate from the cell concentration module (TFF) 724. The cells to be processed are transferred from, e.g., a reservoir in the reagent cartridge to the cell growth module 704 to be cultured until the cells hit a target OD. In this embodiment, the cells are grown in a, e.g., rotating growth vial in a cell growth module separate from the TFF. Once the cells hit the target OD, the cell growth module may cool or freeze the cells for later processing. After growth, the cells may be transferred to the TFF 732, in this instance, a separate module from the cell growth module 704, where buffer or medium exchange is performed, the cells are rendered competent, and the volume of the cells is reduced to a volume optimal for cell transformation in a TFF. Once concentrated, the cells are then transferred to the transformation module 708 in the reagent cartridge (e.g., an electroporation device).

In addition to the reservoir for storing the cells, the reagent cartridge may include a reservoir for storing editing cassettes 716 and a reservoir for storing a vector backbone 718. Both the editing cassettes and the vector backbone are transferred from the reagent cartridge to a nucleic acid assembly module 720, where the editing cassettes are inserted into the vector backbone. The assembled nucleic acids may be transferred into an optional purification module 722 for desalting and/or other purification procedures needed to prepare the assembled nucleic acids for transformation. Once the processes carried out by the assembly/purification module 722 are complete, the assembled nucleic acids are transferred to a transformation module 708, which already contains the cell culture grown to a target OD, rendered competent and concentrated. In the transformation module 708, the nucleic acids are introduced into the cells. Following transformation, the cells are transferred into a combined recovery and editing module 712. As described above, in some embodiments the automated multi-module cell processing instrument 700 is a system that performs gene editing such as an RNA-direct nuclease editing system. For examples of multi-module cell editing instruments, see U.S. Ser. No. 10,253,316, issued 9 Apr. 2019; U.S. Pat. No. 10,329,559, issued 25 Jun. 2019; and U.S. Pat. No. 10,323,242, issued 18 Jun. 2019; and U.S. Ser. No. 16/412,175, filed 14 May 2019; Ser. No. 16/412,195, filed 14 May 2019; and Ser. No. 16/423,289, filed 28 May 2019, all of which are herein incorporated by reference in their entirety. In the recovery and editing module 710, the cells are allowed to recover post-transformation, and the cells express the nuclease and editing oligonucleotides to effect editing in desired genes in the cells.

Following editing, the cells are transferred to a storage module 714, where the cells can be stored at, e.g., 4° C. until the cells are retrieved for further study. The multi-module cell processing instrument is controlled by a processor 750 configured to operate the instrument based on user input, as directed by one or more scripts, or as a combination of user input or a script. The processor 750 may control the timing, duration, temperature, and operations of the various modules of the instrument 700 and the dispensing of reagents from the reagent cartridge. The processor may be programmed with standard protocol parameters from which a user may select, a user may specify one or more parameters manually or one or more scripts associated with the reagent cartridge may specify one or more operations and/or reaction parameters. In addition, the processor may notify the user (e.g., via an application to a smart phone or other device) that the cells have reached a target OD, been rendered competent and concentrated, and/or update the user as to the progress of the cells in the various modules in the multi-module instrument.

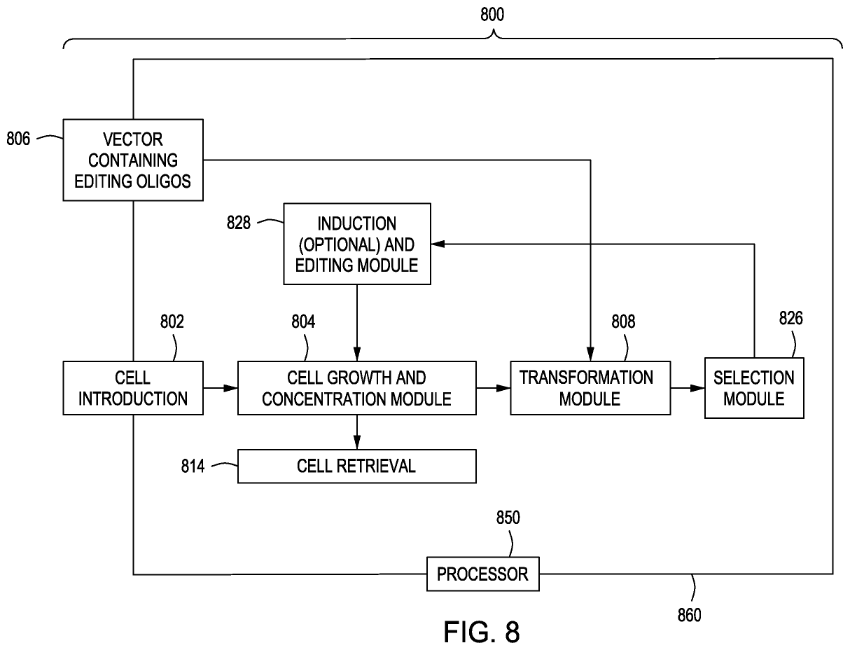

As described above, in one embodiment the automated multi-module cell processing instrument 700 is a nucleic acid-guided nuclease editing system. Multiple nuclease-based systems exist for providing edits into a cell and each can be used in either single editing systems as could be performed in the automated instrument 700 of FIG. 7; and/or sequential editing systems as could be performed in the automated instrument 800 of FIG. 8 described below, e.g., using different nucleic acid-guided nuclease systems sequentially to provide two or more genome edits in a cell; and/or recursive editing.

A second embodiment of a multi-module cell processing instrument is shown in FIG. 8. This embodiment depicts an exemplary system that performs recursive gene editing on a cell population. As with the embodiment shown in FIG. 7, the cell processing system 800 may include a housing 860, a reservoir in a reagent cartridge for storing cells to be transformed or transfected 802, and a TFF module 804. The cells to be transformed are transferred from a reservoir in the reagent cartridge to the TFF module 804 to be cultured until the cells hit a target OD. Once the cells hit the target OD, the TFF module (cell growth and concentration module) 804 renders the cells competent and reduces the volume of the cells. Once the cells have been concentrated to an appropriate volume, the cells are transferred to a transformation module 808. In addition, the assembled nucleic acids are transferred to the transformation module 808, which already contains the cell culture grown to a target OD. In the transformation module 808, the nucleic acids are introduced into the cells. Following transformation, the cells are transferred into a selection module 826.

After selection, the cells may be transferred to an editing module 828 where providing conditions for the cells to edit, e.g., if editing is driven by an inducible promoter. After editing, the cells are transferred back to a TFF module 804 where the edited cells are allowed to grow, and then buffer or medium exchange is performed once again and the cells are rendered competent once again in preparation for transfer to the transformation module 808. Note that in the case of a SWIIN, for example, selection, editing and growth all take place in the same module.

In transformation module 808, the cells are transformed by a second set of editing cassettes (or other type of cassette) and the cycle is repeated until the cells have been transformed and edited by a desired number of, e.g., editing cassettes. As discussed above in relation to FIG. 7, the exemplary multi-module cell processing instrument is controlled by a processor 850 configured to operate the instrument based on user input, or is controlled by one or more scripts, for example, one or more scripts associated with the reagent cartridge. The processor 850 may control the timing, duration, and temperature of various processes, the dispensing of reagents, and other operations of the various modules of the instrument 800. For example, a script or the processor may control the dispensing of cells, reagents, vectors, and editing cassettes; which editing cassettes are used for cell editing and in what order; the time, temperature and other conditions used in the recovery and expression module, the wavelength at which OD is read in the TFF module, the target OD to which the cells are grown, the target time at which the cells will reach the target OD, and/or the volume to which the cells are concentrated. In addition, the processor may be programmed to notify a user (e.g., via an application) as to the progress of the cells in the automated multi-module cell processing instrument.

It should be apparent to one of ordinary skill in the art given the present disclosure that the process described may be recursive and multiplexed; that is, cells may go through the workflow described in relation to FIG. 8, then the resulting edited culture may go through another (or several or many) rounds of additional transformation and editing (e.g., recursive editing) with different editing vectors or vector libraries. For example, the cells from round 1 of editing may be diluted and an aliquot of the edited cells containing vector A can be transformed with either vector B, vector C, vector D and so on for a second round of editing. After round two, an aliquot of each of the double-edited cells may be subjected to a third round of editing, where, e.g., aliquots of all pairwise edit combinations (AB-, AC-, AD-edited cells) are transformed with additional editing vectors, such as editing vectors X, Y, and Z to produce all 3 edit combinations (i.e. ABX, ABY, ABZ, ACX, ACY, ACZ, ADX, ADY, ADZ) and so on. In this process, many permutations and combinations of edits can be executed, leading to very diverse cell populations and cell libraries. In any recursive process, it is advantageous to “cure” the previous engine and editing vectors (or single engine+editing vector in a single vector system). “Curing” is a process in which one or more vectors used in the prior round of editing is eliminated from the transformed cells.

Curing can be accomplished by, e.g., cleaving the vector(s) using a curing plasmid thereby rendering the editing and/or engine vector (or single, combined engine/editing vector) nonfunctional; diluting the vector(s) in the cell population via cell growth (that is, the more growth cycles the cells go through, the fewer daughter cells will retain the editing or engine vector(s)), or by, e.g., utilizing a heat-sensitive origin of replication on the editing or engine vector (or combined engine+editing vector). The conditions for curing will depend on the mechanism used for curing; that is, in this example, how the curing plasmid cleaves the editing and/or engine vector.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: dPCR Readout

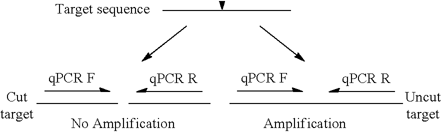

To measure cut activity of each split protein pair, 2 uL (quantity varies due to translation efficiency) from each in-vitro translation reaction are pooled together with 1 uL 300 nM gRNA and 4 uL with 1×NEB cut smart buffer (9 uL total). RNP is allowed to form for 20 minutes followed by addition of 1 uL of 100 nM target DNA (10 nM final). Digestion reactions are incubated at 37° C. for 2 hrs to allow complete digestion and then moved to −20° C. until qPCR reactions are ready. Following digestion, 1 μL of the digestion reaction is used as template for in a qPCR reaction (using SSO advanced kit from Bio Rad and following manufacturer's instructions) to determine the remaining un-cut or intact target DNA concentration. Split protein pairs that exhibit a lack of cleavage in the absence of donor DNA and >90% digestion of input material in the presence of the donor DNA are considered candidates for follow on characterization. Absolute quantification of the % digest completion is determined the fit from calibration curves of titrated undigested target. Controls of purified MAD7-gRNA RNP complex+30 nM gRNA and no-enzyme are used to confirm gRNA activity and establish baselines for further normalization of overall activity donor DNA dependence.

### Example II: Cy3 Cy5 Readout

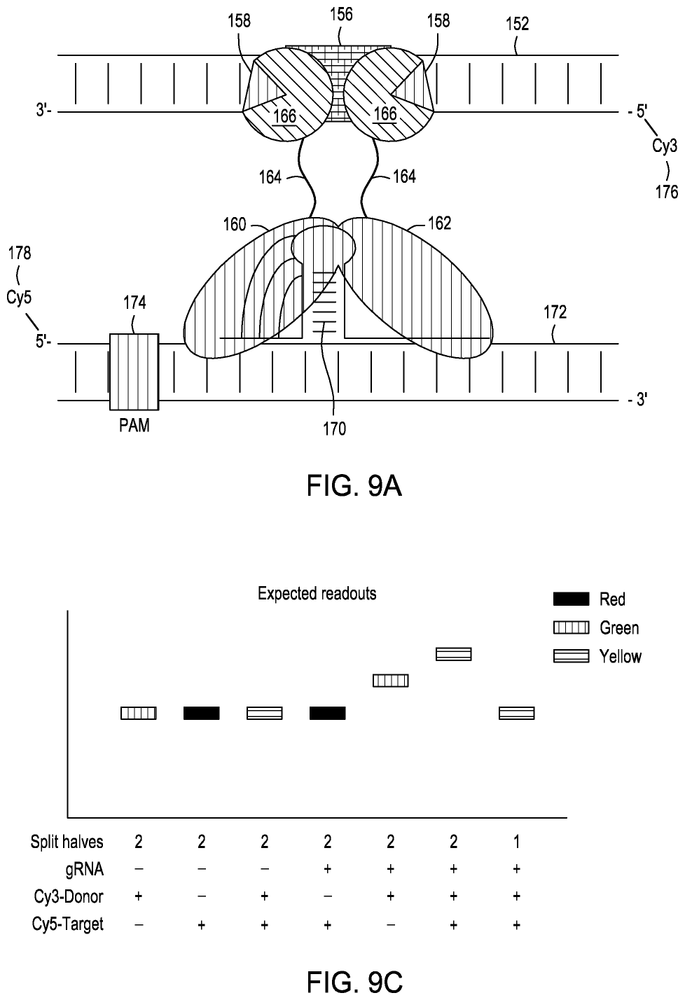

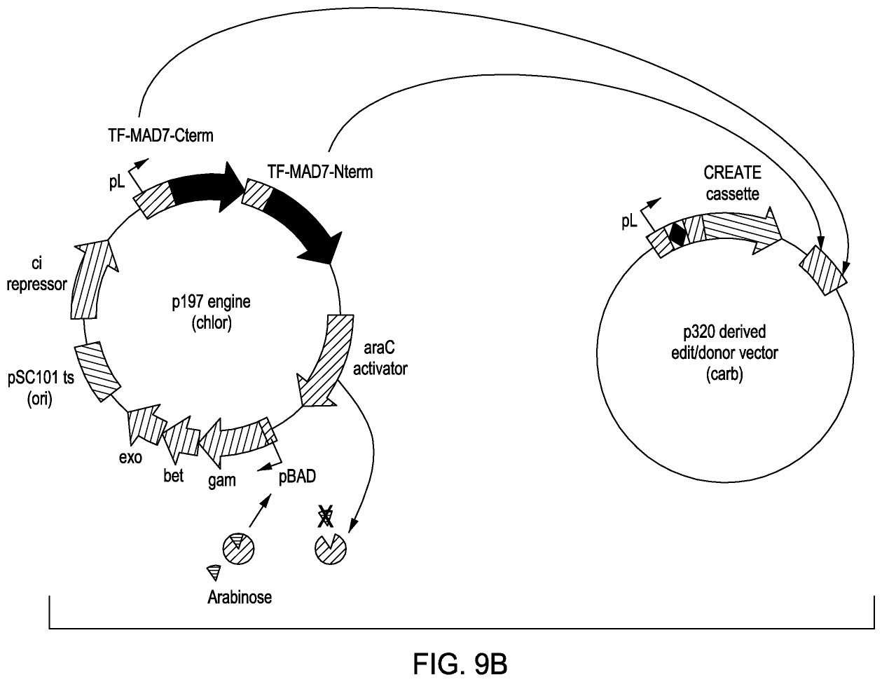

FIG. 9A at top shows the split nuclease tethering system 184 comprising two transcription factor molecules 166 bound to a transcription factor binding site 156 on editing vector 152 and the N-terminal 160 and C-terminal 162 portions or regions of a nuclease bound to gRNA/donor DNA transcript 170, which is bound to target site 172 in a genome comprising a PAM site 174.

FIG. 9B depicts two exemplary vectors—one engine vector and one editing vector—comprising the elements needed for editing in E. coli using a split nuclease tethering system. At left is an engine vector comprising from 11 o-clock continuing clockwise, an inducible pL promoter driving transcription of a MAD7 C-terminal transcription factor construct and a MAD7 N-terminal transcription factor construct (in this example, an Ara^{R} transcription factor is used for both transcription factor constructs); a coding sequence for an AraC activator (e.g., the ligand which causes dimerization the Ara^{R} transcription factors); an inducible pBAD promoter driving the Red recombineering system (the pBAD promoter is induced in the presence of arabinose); the pSC101 temperature sensitive origin of replication (which could be, e.g., used for curing of the engine vector, if desired); a chloramphenicol resistance gene (not shown) and the CI repressor; which is a heat sensitive repressor protein that binds to the pL promoter and silences transcription at low temperatures. At right is an editing vector comprising from 11 o'clock and going clockwise the inducible pL promoter driving transcription of a CREATE cassette comprising the gRNA and donor DNA sequences to be transcribed; the binding site for the dimerized transcription factor; and a coding sequence for the carbenicillin resistance gene.

Table 2 shows the N-terminal and C-terminal split points for MAD7 based on homology modeling. The splits decouple the PAM binding and nuclease binding domains. Again, MAD7 is “split” at a point where there is no spontaneous association of the N-terminal and C-terminal portions in the absence of the ligand, which here is an araC activator, and in the absence of transcription factor binding at the Ara^{R} transcription factor binding site on the editing vector but where there is association of the N-terminal and C-terminal portions of MAD7 and reconstitution of nuclease activity in the presence of the ligand and upon binding of the dimerized Ara^{R} transcription factor at the Ara^{R} transcription factor binding site.

In the experiments herein, the transcription factor Ara^{R} from Bacillus subtilis was used. Ara^{R} is known to bind to eight different operator sites in the bacterial genome and is the key regulatory protein of the L-arabinose metabolism in Bacillus subtilis. Ara^{R} is composed of two independent domains exhibiting different functions and belong to different family of proteins. The smaller N-terminus domain can bind DNA (Ara^{R}-DBD) even in the absence of C-terminus domain. The larger C-terminus domain binds L-arabinose and belongs to LacI/GalR family. Ara^{R} operators are palindromic DNA sequences and the consensus operator is 16 bp in length.

FIG. 9C provides the expected readouts for results in a gel shift application for seven different experimental variations. The MAD7 N-terminal and C-terminal splits shown in Table 2 are both linked to the Ara^{R} transcription factor. The N-terminal and C-terminal transcription factor construct pairs are incubated plus/minus gRNA with donor DNA. The editing vector 152 (donor DNA) is labeled with Cy3 176 and the target nucleic acid 172 is labeled with Cy5 178 (for 176 and 178 see FIG. 9A). The chart at the bottom of FIG. 1C shows the expected readouts (+/−) for the experimental conditions listed at left and the simplified graphic of a gel at top shows expected gel shift for the target site under various conditions. The gel shift indicates binding or association of the editing components and the color readout allows confirmation of the presence of the donor DNA and target in the mix.

In the left-most lane (lane 1), there is no expected shift (binding) in the absence of donor DNA or gRNA and the band is green (Cy3 from the donor DNA alone). In this experiment, the band for the donor can shift without the target since the donor is binding to the two Ara^{R} domains directly. The target DNA shifts only the presence of both the donor and the gRNA. In the second lane, there is no expected shift (binding) in the absence gRNA and donor DNA and the band is red (Cy5 from the target alone). In lane 3, there is no shift in the absence of gRNA but the band is yellow (Cy3 green from the donor DNA combined with Cy5 red from the target). In lane 4 there is no shift in the absence of donor DNA and the band is red (Cy5 from the target alone). In lane 5, there is a shift with the N-terminal and C-terminal constructs associating with the donor DNA and gRNA with the band being green (Cy3 from the donor but without Cy5 from the target). In lane 6 there is a shift showing the association of the N-terminal and C-terminal constructs associating with the donor DNA, gRNA, and target with the band being yellow (Cy3 green from the donor DNA combined with Cy5 red from the target). Finally, lane 7 shows that with only one of the two nuclease constructs present, there is no band shift but the band is yellow (Cy3 green from the donor DNA combined with Cy5 red from the target).

### Example VI: Fully-Automated Singleplex RGN-Directed Editing Run

Singleplex automated genomic editing using MAD7 nuclease was successfully performed with an automated multi-module instrument of the disclosure. See U.S. Pat. No. 9,982,279; and U.S. Ser. No. 16/024,831 filed 30 Jun. 2018; Ser. No. 16/024,816 filed 30 Jun. 2018; Ser. No. 16/147,353 filed 28 Sep. 2018; Ser. No. 16/147,865 filed 30 Sep. 2018; and Ser. No. 16/147,871 filed 30 Jun. 2018.

An ampR plasmid backbone and a lacZ_F172* editing cassette were assembled via Gibson Assembly® into an “editing vector” in an isothermal nucleic acid assembly module included in the automated instrument. lacZ_F172 functionally knocks out the lacZ gene. “lacZ_F172*” indicates that the edit happens at the 172nd residue in the lacZ amino acid sequence. Following assembly, the product was de-salted in the isothermal nucleic acid assembly module using AMPure beads, washed with 80% ethanol, and eluted in buffer. The assembled editing vector and recombineering-ready, electrocompetent E. Coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module), and allowed to recover in SOC medium containing chloramphenicol. Carbenicillin was added to the medium after 1 hour, and the cells were allowed to recover for another 2 hours. After recovery, the cells were held at 4° C. until recovered by the user.

After the automated process and recovery, an aliquot of cells was plated on MacConkey agar base supplemented with lactose (as the sugar substrate), chloramphenicol and carbenicillin and grown until colonies appeared. White colonies represented functionally edited cells, purple colonies represented un-edited cells. All liquid transfers were performed by the automated liquid handling device of the automated multi-module cell processing instrument.

The result of the automated processing was that approximately 1.0E⁻⁰³ total cells were transformed (comparable to conventional benchtop results), and the editing efficiency was 83.5%. The lacZ_172 edit in the white colonies was confirmed by sequencing of the edited region of the genome of the cells. Further, steps of the automated cell processing were observed remotely by webcam and text messages were sent to update the status of the automated processing procedure.

### Example VII: Fully-Automated Recursive Editing Run

Recursive editing was successfully achieved using the automated multi-module cell processing system. An ampR plasmid backbone and a lacZ_V10* editing cassette were assembled via Gibson Assembly® into an “editing vector” in an isothermal nucleic acid assembly module included in the automated system. Similar to the lacZ_F172 edit, the lacZ_V10 edit functionally knocks out the lacZ gene. “lacZ_V10” indicates that the edit happens at amino acid position 10 in the lacZ amino acid sequence. Following assembly, the product was de-salted in the isothermal nucleic acid assembly module using AMPure beads, washed with 80% ethanol, and eluted in buffer. The first assembled editing vector and the recombineering-ready electrocompetent E. Coli cells were transferred into a transformation module for electroporation. The cells and nucleic acids were combined and allowed to mix for 1 minute, and electroporation was performed for 30 seconds. The parameters for the poring pulse were: voltage, 2400 V; length, 5 ms; interval, 50 ms; number of pulses, 1; polarity, +. The parameters for the transfer pulses were: Voltage, 150 V; length, 50 ms; interval, 50 ms; number of pulses, 20; polarity, +/−. Following electroporation, the cells were transferred to a recovery module (another growth module) allowed to recover in SOC medium containing chloramphenicol. Carbenicillin was added to the medium after 1 hour, and the cells were grown for another 2 hours. The cells were then transferred to a centrifuge module and a media exchange was then performed. Cells were resuspended in TB containing chloramphenicol and carbenicillin where the cells were grown to OD600 of 2.7, then concentrated and rendered electrocompetent.

During cell growth, a second editing vector was prepared in an isothermal nucleic acid assembly module. The second editing vector comprised a kanamycin resistance gene, and the editing cassette comprised a galK Y145* edit. If successful, the galK Y145* edit confers on the cells the ability to uptake and metabolize galactose. The edit generated by the galK Y154* cassette introduces a stop codon at the 154th amino acid reside, changing the tyrosine amino acid to a stop codon. This edit makes the galK gene product non-functional and inhibits the cells from being able to metabolize galactose. Following assembly, the second editing vector product was de-salted in the isothermal nucleic acid assembly module using AMPure beads, washed with 80% ethanol, and eluted in buffer. The assembled second editing vector and the electrocompetent E. Coli cells (that were transformed with and selected for the first editing vector) were transferred into a transformation module for electroporation, using the same parameters as detailed above. Following electroporation, the cells were transferred to a recovery module (another growth module), allowed to recover in SOC medium containing carbenicillin. After recovery, the cells were held at 4° C. until retrieved, after which an aliquot of cells were plated on LB agar supplemented with chloramphenicol, and kanamycin. To quantify both lacZ and galK edits, replica patch plates were generated on two media types: 1) MacConkey agar base supplemented with lactose (as the sugar substrate), chloramphenicol, and kanamycin, and 2) MacConkey agar base supplemented with galactose (as the sugar substrate), chloramphenicol, and kanamycin. All liquid transfers were performed by the automated liquid handling device of the automated multi-module cell processing system.

In this recursive editing experiment, 41% of the colonies screened had both the lacZ and galK edits, the results of which were comparable to the double editing efficiencies obtained using a “benchtop” or manual approach.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

TABLE 2
N-terminal and C-terminal MAD7 splits

| SEQ | | | | | | |
|---|---|---|---|---|---|---|
| ID | | Nt | AA | Protein | Nt | AA |
| No. | Seq Name | position | position | Fragment | size | size |
| 4 | MAD7_1630_N | 1887 | 630 | MNNGTNNFQNFIGISSLQKTLRNALIPTETTQQFIVKNGIIKEDELRGE | 1908 | 629 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFD | | |
| 5 | MAD7_1630_C | 1887 | 630 | ITECHDLIDYEKNCIAIHPEWKNEGFDFSDTSTYEDISGFYREVELQGY | 1887 | 636 |
| | | | | KIDWTYISEKDIDLLQEKGQLYLFQIYNKDFSKKSTGNDNLHTMYLKNL | | |
| | | | | FSEENLKDIVLKLNGEAEIFFRKSSIKNPIIHKKGSILVNRTYEAEEKD | | |
| | | | | QFGNIQIVRKNIPENIYQELYKYENDKSDKELSDEAAKLKNVVGHHEAA | | |
| | | | | TNIVKDYRYTYDKYFLHMPITINFKANKTGFINDRILQYIAKEKDLHVI | | |
| | | | | GIDRGERNLIYVSVIDTCGNIVEQKSFNIVNGYDYQIKLKQQEGARQIA | | |
| | | | | RKEWKEIGKIKEIKEGYLSLVIHEISKMVIKYNAIIAMEDLSYGEKKGR | | |
| | | | | EKVERQVYQKFETMLINKLNYLVEKDISITENGGLLKGYQLTYIPDKLK | | |
| | | | | NVGHQCGCIFYVPAAYTSKIDPTTGEVNIFKFKDLTVDAKREFIKKEDS | | |
| | | | | IRYDSEKNLFCFTFDYNNFITQNTVMSKSSWSVYTYGVRIKRREVNGRE | | |
| | | | | SNESDTIDITKDMEKTLEMTDINWRDGHDLRQDIIDYEIVQHIFEIFRL | | |
| | | | | TVQMRNSLSELEDRDYDRLISPVLNENNIFYDSAKAGDALPKDADANGA | | |
| | | | | YCIALKGLYEIKQITENWKEDGKESRDKLKISNKDWFDFIQNKRYL** | | |
| 6 | MAD7_F632_N | 1893 | 632 | MNNGTNNFQNFIGISSLQKTLRNALIPTETTQQFIVKNGIIKEDELRGE | 1902 | 631 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDIT | | |
| 7 | MAD7_F632_C | 1893 | 632 | FCHDLIDYEKNCIAIHPEWKNEGFDFSDTSTYEDISGFYREVELQGYKI | 1893 | 634 |
| | | | | DWTYISEKDIDLLQEKGQLYLFQIYNKDFSKKSTGNDNLHTMYLKNLFS | | |
| | | | | EENLKDIVLKLNGEAEIFFRKSSIKNPIIHKKGSILVNRTYEAEEKDQF | | |
| | | | | GNIQIVRKNIPENIYQELYKYENDKSDKELSDEAAKLKNVVGHHEAATN | | |
| | | | | IVKDYRYTYDKYFLHMPITINFKANKTGFINDRILQYIAKEKDLHVIGI | | |
| | | | | DRGERNLIYVSVIDTCGNIVEQKSFNIVNGYDYQIKLKQQEGARQIARK | | |
| | | | | EWKEIGKIKEIKEGYLSLVIHEISKMVIKYNAIIAMEDLSYGEKKGREK | | |
| | | | | VERQVYQKFETMLINKLNYLVEKDISITENGGLLKGYQLTYIPDKLKNV | | |
| | | | | GHQCGCIFYVPAAYTSKIDPTTGEVNIFKFKDLTVDAKREFIKKEDSIR | | |
| | | | | YDSEKNLECFTEDYNNFITQNTVMSKSSWSVYTYGVRIKRREVNGRESN | | |
| | | | | ESDTIDITKDMEKTLEMTDINWRDGHDLRQDIIDYEIVQHIFEIFRLTV | | |
| | | | | QMRNSLSELEDRDYDRLISPVLNENNIFYDSAKAGDALPKDADANGAYC | | |
| | | | | IALKGLYEIKQITENWKEDGKESRDKLKISNKDWFDFIQNKRYL** | | |
| 8 | MAD7_H634_N | 1899 | 634 | MNNGTNNFQNFIGISSLQKTLRNALIPTETTQQFIVKNGIIKEDELRGE | 1896 | 633 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITFC | | |
| 9 | MAD7_H634_C | 1899 | 634 | HDLIDYEKNCIAIHPEWKNEGFDFSDTSTYEDISGFYREVELQGYKIDW | 1899 | 632 |
| | | | | TYISEKDIDLLQEKGQLYLFQIYNKDFSKKSTGNDNLHTMYLKNLFSEE | | |
| | | | | NLKDIVLKLNGEAEIFFRKSSIKNPIIHKKGSILVNRTYEAEEKDQFGN | | |
| | | | | IQIVRKNIPENIYQELYKYENDKSDKELSDEAAKLKNVVGHHEAATNIV | | |
| | | | | KDYRYTYDKYFLHMPITINFKANKTGFINDRILQYIAKEKDLHVIGIDR | | |
| | | | | GERNLIYVSVIDTCGNIVEQKSFNIVNGYDYQIKLKQQEGARQIARKEW | | |
| | | | | KEIGKIKEIKEGYLSLVIHEISKMVIKYNAIIAMEDLSYGEKKGREKVE | | |
| | | | | RQVYQKFETMLINKLNYLVEKDISITENGGLLKGYQLTYIPDKLKNVGH | | |
| | | | | QCGCIFYVPAAYTSKIDPTTGEVNIFKFKDLTVDAKREFIKKEDSIRYD | | |
| | | | | SEKNLFCFTFDYNNFITQNTVMSKSSWSVYTYGVRIKRREVNGRESNES | | |
| | | | | DTIDITKDMEKTLEMTDINWRDGHDLRQDIIDYEIVQHIFEIFRLTVQM | | |
| | | | | RNSLSELEDRDYDRLISPVLNENNIFYDSAKAGDALPKDADANGAYCIA | | |
| | | | | LKGLYEIKQITENWKEDGKESRDKLKISNKDWFDFIQNKRYL** | | |
| 10 | MAD7_L636_N | 1905 | 636 | MNNGTNNFQNFIGISSLQKTLRNALIPTETTQQFIVKNGIIKEDELRGE | 1890 | 635 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHD | | |
| 11 | MAD7_L636_C | 1905 | 636 | DYEKNCIAIHPEWKNEGFDFSDTSTYEDISGFYREVELQGYKIDWTYIS | 1905 | 630 |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNYLVEKDISITENGGLLKGYQLTYIPDKLKNVGHQCGC | | |
| | | | | IFYVPAAYTSKIDPTTGEVNIFKFKDLTVDAKREFIKKEDSIRYDSEKN | | |
| | | | | LFCFTFDYNNFITQNTVMSKSSWSVYTYGVRIKRREVNGRESNESDTID | | |
| | | | | ITKDMEKTLEMTDINWRDGHDLRQDIIDYEIVQHIFEIFRLTVQMRNSL | | |
| | | | | SELEDRDYDRLISPVLNENNIFYDSAKAGDALPKDADANGAYCIALKGL | | |
| | | | | YEIKQITENWKEDGKESRDKLKISNKDWFDFIQNKRYL** | | |
| 12 | MAD7_D638_N | 1911 | 638 | MNNGTNNFQNFIGISSLQKTLRNALIPTETTQQFIVKNGIIKEDELRGE | 1884 | 637 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DY | | |
| 13 | MAD7_D638_C | 1911 | 638 | FKNCIAIHPEWKNEGFDFSDTSTYEDISGFYREVELQGYKIDWTYISEK | 1911 | 628 |
| | | | | DIDLLQEKGQLYLFQIYNKDFSKKSTGNDNLHTMYLKNLFSEENLKDIV | | |
| | | | | LKLNGEAEIFFRKSSIKNPIIHKKGSILVNRTYEAEEKDQFGNIQIVRK | | |
| | | | | NIPENIYQELYKYENDKSDKELSDEAAKLKNVVGHHEAATNIVKDYRYT | | |
| | | | | YDKYFLHMPITINFKANKTGFINDRILQYIAKEKDLHVIGIDRGERNLI | | |
| | | | | YVSVIDTCGNIVEQKSFNIVNGYDYQIKLKQQEGARQIARKEWKEIGKI | | |
| | | | | KEIKEGYLSLVIHEISKMVIKYNAIIAMEDLSYGEKKGRFKVERQVYQK | | |
| | | | | FETMLINKLNYLVEKDISITENGGLLKGYQLTYIPDKLKNVGHQCGCIF | | |
| | | | | YVPAAYTSKIDPTTGEVNIFKFKDLTVDAKREFIKKEDSIRYDSEKNLF | | |
| | | | | CFTFDYNNFITQNTVMSKSSWSVYTYGVRIKRREVNGRESNESDTIDIT | | |
| | | | | KDMEKTLEMTDINWRDGHDLRQDIIDYEIVQHIFEIFRLTVQMRNSLSE | | |
| | | | | LEDRDYDRLISPVLNENNIFYDSAKAGDALPKDADANGAYCIALKGLYE | | |
| | | | | IKQITENWKEDGKESRDKLKISNKDWFDFIQNKRYL** | | |
| 14 | MAD7_F640_N | 1917 | 640 | MNNGTNNFQNFIGISSLQKTLRNALIPTETTQQFIVKNGIIKEDELRGE | 1878 | 639 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYFK | | |
| 15 | MAD7_F640_C | 1917 | 640 | NCIAIHPEWKNEGFDFSDTSTYEDISGFYREVELQGYKIDWTYISEKDI | 1917 | 626 |
| | | | | DLLQEKGQLYLFQIYNKDFSKKSTGNDNLHTMYLKNLFSEENLKDIVLK | | |
| | | | | LNGEAEIFFRKSSIKNPIIHKKGSILVNRTYEAEEKDQFGNIQIVRKNI | | |
| | | | | PENIYQELYKYENDKSDKELSDEAAKLKNVVGHHEAATNIVKDYRYTYD | | |
| | | | | KYFLHMPITINFKANKTGFINDRILQYIAKEKDLHVIGIDRGERNLIYV | | |
| | | | | SVIDTCGNIVEQKSFNIVNGYDYQIKLKQQEGARQIARKEWKEIGKIKE | | |
| | | | | IKEGYLSLVIHEISKMVIKYNAIIAMEDLSYGEKKGRFKVERQVYQKFE | | |
| | | | | TMLINKLNYLVFKDISITENGGLLKGYQLTYIPDKLKNVGHQCGCIFYV | | |
| | | | | PAAYTSKIDPTTGEVNIFKFKDLTVDAKREFIKKEDSIRYDSEKNLECF | | |
| | | | | TEDYNNFITQNTVMSKSSWSVYTYGVRIKRRFVNGRESNESDTIDITKD | | |
| | | | | MEKTLEMTDINWRDGHDLRQDIIDYEIVQHIFEIFRLTVQMRNSLSELE | | |
| | | | | DRDYDRLISPVLNENNIFYDSAKAGDALPKDADANGAYCIALKGLYEIK | | |
| | | | | QITENWKEDGKESRDKLKISNKDWFDFIQNKRYL** | | |
| 16 | MAD7_N642_N | 1923 | 642 | MNNGTNNFQNFIGISSLQKTLRNALIPTETTQQFIVKNGIIKEDELRGE | 1872 | 641 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNC | | |
| 17 | MAD7_N642_C | 1923 | 642 | IAIHPEWKNEGFDFSDTSTYEDISGFYREVELQGYKIDWTYISEKDIDL | 1923 | 624 |
| | | | | LQEKGQLYLFQIYNKDFSKKSTGNDNLHTMYLKNLFSEENLKDIVLKLN | | |
| | | | | GEAEIFFRKSSIKNPIIHKKGSILVNRTYEAEEKDQFGNIQIVRKNIPE | | |
| | | | | NIYQELYKYENDKSDKELSDEAAKLKNVVGHHEAATNIVKDYRYTYDKY | | |
| | | | | FLHMPITINFKANKTGFINDRILQYIAKEKDLHVIGIDRGERNLIYVSV | | |
| | | | | IDTCGNIVEQKSFNIVNGYDYQIKLKQQEGARQIARKEWKEIGKIKEIK | | |
| | | | | EGYLSLVIHEISKMVIKYNAIIAMEDLSYGEKKGRFKVERQVYQKFETM | | |
| | | | | LINKLNYLVEKDISITENGGLLKGYQLTYIPDKLKNVGHQCGCIFYVPA | | |
| | | | | AYTSKIDPTTGEVNIFKFKDLTVDAKREFIKKEDSIRYDSEKNLFCFTF | | |
| | | | | DYNNFITQNTVMSKSSWSVYTYGVRIKRREVNGRESNESDTIDITKDME | | |
| | | | | KTLEMTDINWRDGHDLRQDIIDYEIVQHIFEIFRLTVQMRNSLSELEDR | | |
| | | | | DYDRLISPVLNENNIFYDSAKAGDALPKDADANGAYCIALKGLYEIKQI | | |
| | | | | TENWKEDGKESRDKLKISNKDWFDFIQNKRYL** | | |
| 18 | MAD7_I644_N | 1929 | 644 | MNNGTNNFQNFIGISSLQKTLRNALIPTETTQQFIVKNGIIKEDELRGE | 1866 | 643 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIA | | |
| 19 | MAD7_I644_C | 1929 | 644 | IAIHPEWKNEGFDFSDTSTYEDISGFYREVELQGYKIDWTYISEKDIDL | 1929 | 622 |
| | | | | LQEKGQLYLFQIYNKDFSKKSTGNDNLHTMYLKNLFSEENLKDIVLKLN | | |
| | | | | GEAEIFFRKSSIKNPIIHKKGSILVNRTYEAEEKDQFGNIQIVRKNIPE | | |
| | | | | NIYQELYKYENDKSDKELSDEAAKLKNVVGHHEAATNIVKDYRYTYDKY | | |
| | | | | FLHMPITINFKANKTGFINDRILQYIAKEKDLHVIGIDRGERNLIYVSV | | |
| | | | | IDTCGNIVEQKSFNIVNGYDYQIKLKQQEGARQIARKEWKEIGKIKEIK | | |
| | | | | EGYLSLVIHEISKMVIKYNAIIAMEDLSYGEKKGRFKVERQVYQKFETM | | |
| | | | | LINKLNYLVEKDISITENGGLLKGYQLTYIPDKLKNVGHQCGCIFYVPA | | |
| | | | | AYTSKIDPTTGEVNIFKFKDLTVDAKREFIKKEDSIRYDSEKNLFCFTF | | |

DYNNFITQNTVMSKSSWSVYTYGVRIKRREVNGRESNESDTIDITKDME

| | | | | | | |
|---|---|---|---|---|---|---|
| | | | | KTLEMTDINWRDGHDLRQDIIDYEIVQHIFEIFRLTVQMRNSLSELEDR | | |
| | | | | DYDRLISPVLNENNIFYDSAKAGDALPKDADANGAYCIALKGLYEIKQI | | |
| | | | | TENWKEDGKESRDKLKISNKDWFDFIQNKRYL** | | |
| 20 | MAD7_I646_N | 1935 | 646 | MNNGTNNFQNFIGISSLQKTLRNALIPTETTQQFIVKNGIIKEDELRGE | 1860 | 645 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIA | | |
| 21 | MAD7_I646_C | 1935 | 646 | IHPEWKNEGFDFSDTSTYEDISGFYREVELQGYKIDWTYISEKDIDLLQ | 1935 | 620 |
| | | | | EKGQLYLFQIYNKDFSKKSTGNDNLHTMYLKNLFSEENLKDIVLKLNGE | | |
| | | | | AEIFFRKSSIKNPIIHKKGSILVNRTYEAEEKDQFGNIQIVRKNIPENI | | |
| | | | | YQELYKYENDKSDKELSDEAAKLKNVVGHHEAATNIVKDYRYTYDKYFL | | |
| | | | | HMPITINFKANKTGFINDRILQYIAKEKDLHVIGIDRGERNLIYVSVID | | |
| | | | | TCGNIVEQKSFNIVNGYDYQIKLKQQEGARQIARKEWKEIGKIKEIKEG | | |
| | | | | YLSLVIHEISKMVIKYNAIIAMEDLSYGEKKGRFKVERQVYQKFETMLI | | |
| | | | | NKLNYLVEKDISITENGGLLKGYQLTYIPDKLKNVGHQCGCIFYVPAAY | | |
| | | | | TSKIDPTTGEVNIFKFKDLTVDAKREFIKKEDSIRYDSEKNLFCFTFDY | | |
| | | | | NNFITQNTVMSKSSWSVYTYGVRIKRREVNGRFSNESDTIDITKDMEKT | | |
| | | | | LEMTDINWRDGHDLRQDIIDYEIVQHIFEIFRLTVQMRNSLSELEDRDY | | |
| | | | | DRLISPVLNENNIFYDSAKAGDALPKDADANGAYCIALKGLYEIKQITE | | |
| | | | | NWKEDGKESRDKLKISNKDWFDFIQNKRYL** | | |
| 22 | MAD7_P648_N | 1941 | 648 | MNNGTNNFQNFIGISSLQKTLRNALIPTETTQQFIVKNGIIKEDELRGE | 1854 | 647 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIH | | |
| 23 | MAD7_P648_C | 1941 | 648 | PEWKNEGFDFSDTSTYEDISGFYREVELQGYKIDWTYISEKDIDLLQEK | 1941 | 618 |
| | | | | GQLYLFQIYNKDFSKKSTGNDNLHTMYLKNLFSEENLKDIVLKLNGEAE | | |
| | | | | IFFRKSSIKNPIIHKKGSILVNRTYEAEEKDQFGNIQIVRKNIPENIYQ | | |
| | | | | ELYKYENDKSDKELSDEAAKLKNVVGHHEAATNIVKDYRYTYDKYFLHM | | |
| | | | | PITINFKANKTGFINDRILQYIAKEKDLHVIGIDRGERNLIYVSVIDTC | | |
| | | | | GNIVEQKSFNIVNGYDYQIKLKQQEGARQIARKEWKEIGKIKEIKEGYL | | |
| | | | | SLVIHEISKMVIKYNAIIAMEDLSYGEKKGRFKVERQVYQKFETMLINK | | |
| | | | | LNYLVEKDISITENGGLLKGYQLTYIPDKLKNVGHQCGCIFYVPAAYTS | | |
| | | | | KIDPTTGEVNIFKFKDLTVDAKREFIKKEDSIRYDSEKNLFCFTFDYNN | | |
| | | | | FITQNTVMSKSSWSVYTYGVRIKRREVNGRESNESDTIDITKDMEKTLE | | |
| | | | | MTDINWRDGHDLRQDIIDYEIVQHIFEIFRLTVQMRNSLSELEDRDYDR | | |
| | | | | LISPVLNENNIFYDSAKAGDALPKDADANGAYCIALKGLYEIKQITENW | | |
| | | | | KEDGKESRDKLKISNKDWFDFIQNKRYL** | | |
| 24 | MAD7_N848_N | 2541 | 848 | MNNGTNNFQNFIGISSLQKTLRNALIPTETTQQFIVKNGIIKEDELRGE | 1254 | 847 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITI | | |
| 25 | MAD7_N848_C | 2541 | 848 | NFKANKTGFINDRILQYIAKEKDLHVIGIDRGERNLIYVSVIDTCGNIV | 2541 | 418 |
| | | | | EQKSFNIVNGYDYQIKLKQQEGARQIARKEWKEIGKIKEIKEGYLSLVI | | |
| | | | | HEISKMVIKYNAIIAMEDLSYGEKKGRFKVERQVYQKFETMLINKLNYL | | |
| | | | | VEKDISITENGGLLKGYQLTYIPDKLKNVGHQCGCIFYVPAAYTSKIDP | | |
| | | | | TTGEVNIFKFKDLTVDAKREFIKKEDSIRYDSEKNLECETEDYNNFITQ | | |
| | | | | NTVMSKSSWSVYTYGVRIKRREVNGRESNESDTIDITKDMEKTLEMTDI | | |
| | | | | NWRDGHDLRQDIIDYEIVQHIFEIFRLTVQMRNSLSELEDRDYDRLISP | | |
| | | | | VLNENNIFYDSAKAGDALPKDADANGAYCIALKGLYEIKQITENWKEDG | | |
| | | | | KESRDKLKISNKDWFDFIQNKRYL** | | |
| 26 | MAD7_K850_N | 2547 | 850 | MNNGTNNFQNFIGISSLQKTLRNALIPTETTQQFIVKNGIIKEDELRGE | 1248 | 849 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINF | | |
| 27 | MAD7_K850_C | 2547 | 850 | KANKTGFINDRILQYIAKEKDLHVIGIDRGERNLIYVSVIDTCGNIVEQ | 2547 | 416 |
| | | | | KSFNIVNGYDYQIKLKQQEGARQIARKEWKEIGKIKEIKEGYLSLVIHE | | |
| | | | | ISKMVIKYNAIIAMEDLSYGFKKGRFKVERQVYQKFETMLINKLNYLVE | | |
| | | | | KDISITENGGLLKGYQLTYIPDKLKNVGHQCGCIFYVPAAYTSKIDPTT | | |
| | | | | GEVNIFKFKDLTVDAKREFIKKEDSIRYDSEKNLFCFTFDYNNFITQNT | | |
| | | | | VMSKSSWSVYTYGVRIKRREVNGRESNESDTIDITKDMEKTLEMTDINW | | |
| | | | | RDGHDLRQDIIDYEIVQHIFEIFRLTVQMRNSLSELEDRDYDRLISPVL | | |
| | | | | NENNIFYDSAKAGDALPKDADANGAYCIALKGLYEIKQITENWKEDGKE | | |
| | | | | SRDKLKISNKDWFDFIQNKRYL** | | |
| 28 | MAD7_N852_N | 2553 | 852 | MNNGTNNFQNFIGISSLQKTLRNALIPTETTQQFIVKNGIIKEDELRGE | 1242 | 851 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKA | | |
| 29 | MAD7_N852_C | 2553 | 852 | NKTGFINDRILQYIAKEKDLHVIGIDRGERNLIYVSVIDTCGNIVEQKS | 2553 | 414 |
| | | | | FNIVNGYDYQIKLKQQEGARQIARKEWKEIGKIKEIKEGYLSLVIHEIS | | |
| | | | | KMVIKYNAIIAMEDLSYGEKKGRFKVERQVYQKFETMLINKLNYLVEKD | | |
| | | | | ISITENGGLLKGYQLTYIPDKLKNVGHQCGCIFYVPAAYTSKIDPTTGE | | |
| | | | | VNIFKFKDLTVDAKREFIKKEDSIRYDSEKNLFCFTFDYNNFITQNTVM | | |
| | | | | SKSSWSVYTYGVRIKRREVNGRESNESDTIDITKDMEKTLEMTDINWRD | | |
| | | | | GHDLRQDIIDYEIVQHIFEIFRLTVQMRNSLSELEDRDYDRLISPVLNE | | |
| | | | | NNIFYDSAKAGDALPKDADANGAYCIALKGLYEIKQITENWKEDGKESR | | |
| | | | | DKLKISNKDWFDFIQNKRYL** | | |
| 30 | MAD7_T854_N | 2559 | 854 | MNNGTNNFQNFIGISSLQKTLRNALIPTETTQQFIVKNGIIKEDELRGE | 1236 | 853 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANK | | |
| 31 | MAD7_T854_C | 2559 | 854 | TGFINDRILQYIAKEKDLHVIGIDRGERNLIYVSVIDTCGNIVEQKSFN | 2559 | 412 |
| | | | | IVNGYDYQIKLKQQEGARQIARKEWKEIGKIKEIKEGYLSLVIHEISKM | | |
| | | | | VIKYNAIIAMEDLSYGEKKGRFKVERQVYQKFETMLINKLNYLVEKDIS | | |
| | | | | ITENGGLLKGYQLTYIPDKLKNVGHQCGCIFYVPAAYTSKIDPTTGEVN | | |
| | | | | IFKFKDLTVDAKREFIKKEDSIRYDSEKNLFCFTFDYNNFITQNTVMSK | | |
| | | | | SSWSVYTYGVRIKRREVNGRESNESDTIDITKDMEKTLEMTDINWRDGH | | |
| | | | | DLRQDIIDYEIVQHIFEIFRLTVQMRNSLSELEDRDYDRLISPVLNENN | | |
| | | | | IFYDSAKAGDALPKDADANGAYCIALKGLYEIKQITENWKEDGKESRDK | | |
| | | | | LKISNKDWFDFIQNKRYL** | | |
| 32 | MAD7_N992_N | 2973 | 992 | MNNGTNNFQNFIGISSLQKTLRNALIPTETTQQFIVKNGIIKEDELRGE | 822 | 991 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKL | | |
| 33 | MAD7_N992_C | 2973 | 992 | NYLVEKDISITENGGLLKGYQLTYIPDKLKNVGHQCGCIFYVPAAYTSK | 2973 | 274 |
| | | | | IDPTTGEVNIFKFKDLTVDAKREFIKKEDSIRYDSEKNLFCFTFDYNNF | | |
| | | | | ITQNTVMSKSSWSVYTYGVRIKRREVNGRESNESDTIDITKDMEKTLEM | | |
| | | | | TDINWRDGHDLRQDIIDYEIVQHIFEIFRLTVQMRNSLSELEDRDYDRL | | |
| | | | | ISPVLNENNIFYDSAKAGDALPKDADANGAYCIALKGLYEIKQITENWK | | |
| | | | | EDGKESRDKLKISNKDWFDFIQNKRYL** | | |
| 34 | MAD7_L994_N | 2979 | 994 | MNNGTNNFQNFIGISSLQKTLRNALIPTETTQQFIVKNGIIKEDELRGE | 816 | 993 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNY | | |
| 35 | MAD7_L994_C | 2979 | 994 | LVEKDISITENGGLLKGYQLTYIPDKLKNVGHQCGCIFYVPAAYTSKID | 2979 | 272 |
| | | | | PTTGEVNIFKFKDLTVDAKREFIKKEDSIRYDSEKNLFCFTFDYNNFIT | | |
| | | | | QNTVMSKSSWSVYTYGVRIKRREVNGRESNESDTIDITKDMEKTLEMTD | | |
| | | | | INWRDGHDLRQDIIDYEIVQHIFEIFRLTVQMRNSLSELEDRDYDRLIS | | |
| | | | | PVLNENNIFYDSAKAGDALPKDADANGAYCIALKGLYEIKQITENWKED | | |
| | | | | GKESRDKLKISNKDWFDFIQNKRYL** | | |
| 36 | MAD7_F996_N | 2985 | 996 | MNNGTNNFQNFIGISSLQKTLRNALIPTETTQQFIVKNGIIKEDELRGE | 810 | 995 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNYLV | | |
| 37 | MAD7_F996_C | 2985 | 996 | FKDISITENGGLLKGYQLTYIPDKLKNVGHQCGCIFYVPAAYTSKIDPT | 2985 | 270 |
| | | | | TGEVNIFKFKDLTVDAKREFIKKEDSIRYDSEKNLECFTEDYNNFITQN | | |
| | | | | TVMSKSSWSVYTYGVRIKRRFVNGRESNESDTIDITKDMEKTLEMTDIN | | |
| | | | | WRDGHDLRQDIIDYEIVQHIFEIFRLTVQMRNSLSELEDRDYDRLISPV | | |
| | | | | LNENNIFYDSAKAGDALPKDADANGAYCIALKGLYEIKQITENWKEDGK | | |
| | | | | ESRDKLKISNKDWFDFIQNKRYL** | | |
| 38 | MAD7_D998_N | 2991 | 998 | MNNGTNNFQNFIGISSLQKTLRNALIPTETTQQFIVKNGIIKEDELRGE | 804 | 997 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNYLVEK | | |
| 39 | MAD7_D998_C | 2991 | 998 | DISITENGGLLKGYQLTYIPDKLKNVGHQCGCIFYVPAAYTSKIDPTTG | 2991 | 268 |
| | | | | EVNIFKFKDLTVDAKREFIKKEDSIRYDSEKNLFCFTFDYNNFITQNTV | | |
| | | | | MSKSSWSVYTYGVRIKRREVNGRESNESDTIDITKDMEKTLEMTDINWR | | |
| | | | | DGHDLRQDIIDYEIVQHIFEIFRLTVQMRNSLSELEDRDYDRLISPVLN | | |
| | | | | ENNIFYDSAKAGDALPKDADANGAYCIALKGLYEIKQITENWKEDGKES | | |
| | | | | RDKLKISNKDWFDFIQNKRYL** | | |
| 40 | MAD7_S1000_N | 2997 | 1000 | MNNGTNNFQNFIGISSLQKTLRNALIPTETTQQFIVKNGIIKEDELRGE | 798 | 999 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNYLVEKDI | | |
| 41 | MAD7_S1000_C | 2997 | 1000 | SITENGGLLKGYQLTYIPDKLKNVGHQCGCIFYVPAAYTSKIDPTTGEV | 2997 | 266 |
| | | | | NIFKFKDLTVDAKREFIKKEDSIRYDSEKNLFCFTFDYNNFITQNTVMS | | |
| | | | | KSSWSVYTYGVRIKRREVNGRESNESDTIDITKDMEKTLEMTDINWRDG | | |
| | | | | HDLRQDIIDYEIVQHIFEIFRLTVQMRNSLSELEDRDYDRLISPVLNEN | | |
| | | | | NIFYDSAKAGDALPKDADANGAYCIALKGLYEIKQITENWKEDGKESRD | | |
| | | | | KLKISNKDWFDFIQNKRYL** | | |
| 42 | MAD7_T1002_N | 3003 | 1002 | MNNGTNNFQNFIGISSLQKTLRNALIPTETTQQFIVKNGIIKEDELRGE | 792 | 1001 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNYLVEKDISI | | |
| 43 | MAD7_T1002_C | 3003 | 1002 | TENGGLLKGYQLTYIPDKLKNVGHQCGCIFYVPAAYTSKIDPTTGEVNI | 3003 | 264 |
| | | | | FKFKDLTVDAKREFIKKEDSIRYDSEKNLFCFTFDYNNFITQNTVMSKS | | |
| | | | | SWSVYTYGVRIKRREVNGRFSNESDTIDITKDMEKTLEMTDINWRDGHD | | |
| | | | | LRQDIIDYEIVQHIFEIFRLTVQMRNSLSELEDRDYDRLISPVLNENNI | | |
| | | | | FYDSAKAGDALPKDADANGAYCIALKGLYEIKQITENWKEDGKESRDKL | | |
| | | | | KISNKDWFDFIQNKRYL** | | |
| 44 | MAD7_N1004_N | 3009 | 1004 | MNNGTNNFQNFIGISSLQKTLRNALIPTETTQQFIVKNGIIKEDELRGE | 786 | 1003 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNYLVEKDISITE | | |
| 45 | MAD7_N1004_C | 3009 | 1004 | NGGLLKGYQLTYIPDKLKNVGHQCGCIFYVPAAYTSKIDPTTGEVNIFK | 3009 | 262 |
| | | | | FKDLTVDAKREFIKKEDSIRYDSEKNLFCFTFDYNNFITQNTVMSKSSW | | |
| | | | | SVYTYGVRIKRREVNGRESNESDTIDITKDMEKTLEMTDINWRDGHDLR | | |
| | | | | QDIIDYEIVQHIFEIFRLTVQMRNSLSELEDRDYDRLISPVLNENNIFY | | |
| | | | | DSAKAGDALPKDADANGAYCIALKGLYEIKQITENWKEDGKESRDKLKI | | |
| | | | | SNKDWFDFIQNKRYL** | | |
| 46 | MAD7_G1006_N | 3015 | 1006 | MNNGTNNFQNFIGISSLQKTLRNALIPTETTQQFIVKNGIIKEDELRGE | 780 | 1005 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNYLVEKDISITENG | | |
| 47 | MAD7_G1006_C | 3015 | 1006 | GLLKGYQLTYIPDKLKNVGHQCGCIFYVPAAYTSKIDPTTGEVNIFKFK | 3015 | 260 |
| | | | | DLTVDAKREFIKKEDSIRYDSEKNLFCFTFDYNNFITQNTVMSKSSWSV | | |
| | | | | YTYGVRIKRREVNGRESNESDTIDITKDMEKTLEMTDINWRDGHDLRQD | | |
| | | | | IIDYEIVQHIFEIFRLTVQMRNSLSELEDRDYDRLISPVLNENNIFYDS | | |
| | | | | AKAGDALPKDADANGAYCIALKGLYEIKQITENWKEDGKESRDKLKISN | | |
| | | | | KDWFDFIQNKRYL** | | |
| 48 | MAD7_L1008_N | 3021 | 1008 | MNNGTNNFQNFIGISSLQKTLRNALIPTETTQQFIVKNGIIKEDELRGE | 774 | 1007 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNYLVEKDISITENGGL | | |
| 49 | MAD7_L1008_C | 3021 | 1008 | LKGYQLTYIPDKLKNVGHQCGCIFYVPAAYTSKIDPTTGEVNIFKFKDL | 3021 | 258 |
| | | | | TVDAKREFIKKEDSIRYDSEKNLFCFTFDYNNFITQNTVMSKSSWSVYT | | |
| | | | | YGVRIKRREVNGRESNESDTIDITKDMEKTLEMTDINWRDGHDLRQDII | | |
| | | | | DYEIVQHIFEIFRLTVQMRNSLSELEDRDYDRLISPVLNENNIFYDSAK | | |
| | | | | AGDALPKDADANGAYCIALKGLYEIKQITENWKEDGKFSRDKLKISNKD | | |
| | | | | WFDFIQNKRYL** | | |
| 50 | MAD7_G1010_N | 3027 | 1010 | MNNGTNNFQNFIGISSLQKTLRNALIPTETTQQFIVKNGIIKEDELRGE | 768 | 1009 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNYLVEKDISITENGGLLK | | |
| 51 | MAD7_G1010_C | 3027 | 1010 | GYQLTYIPDKLKNVGHQCGCIFYVPAAYTSKIDPTTGEVNIFKFKDLTV | 3027 | 256 |
| | | | | DAKREFIKKEDSIRYDSEKNLECFTEDYNNFITQNTVMSKSSWSVYTYG | | |
| | | | | VRIKRREVNGRESNESDTIDITKDMEKTLEMTDINWRDGHDLRQDIIDY | | |
| | | | | EIVQHIFEIFRLTVQMRNSLSELEDRDYDRLISPVLNENNIFYDSAKAG | | |
| | | | | DALPKDADANGAYCIALKGLYEIKQITENWKEDGKESRDKLKISNKDWF | | |
| | | | | DFIQNKRYL** | | |
| 52 | MAD7_01012_N | 3033 | 1012 | MNNGTNNFQNFIGISSLQKTLRNALIPTETTQQFIVKNGIIKEDELRGE | 762 | 1011 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNYLVEKDISITENGGLLKGY | | |
| 53 | MAD7_01012_C | 3033 | 1012 | QLTYIPDKLKNVGHQCGCIFYVPAAYTSKIDPTTGEVNIFKFKDLTVDA | 3033 | 254 |
| | | | | KREFIKKEDSIRYDSEKNLFCFTFDYNNFITQNTVMSKSSWSVYTYGVR | | |
| | | | | IKRREVNGRESNESDTIDITKDMEKTLEMTDINWRDGHDLRQDIIDYEI | | |
| | | | | VQHIFEIFRLTVQMRNSLSELEDRDYDRLISPVLNENNIFYDSAKAGDA | | |
| | | | | LPKDADANGAYCIALKGLYEIKQITENWKEDGKESRDKLKISNKDWFDF | | |
| | | | | IQNKRYL** | | |
| 54 | MAD7_T1014_N | 3039 | 1014 | MNNGTNNFQNFIGISSLQKTLRNALIPTETTQQFIVKNGIIKEDELRGE | 756 | 1013 |
| | | | | NRQILKDIMDDYYRGFISETLSSIDDIDWTSLFEKMEIQLKNGDNKDTL | | |
| | | | | IKEQTEYRKAIHKKFANDDRFKNMFSAKLISDILPEEVIHNNNYSASEK | | |
| | | | | EEKTQVIKLFSRFATSFKDYEKNRANCFSADDISSSSCHRIVNDNAEIF | | |
| | | | | FSNALVYRRIVKSLSNDDINKISGDMKDSLKEMSLEEIYSYEKYGEFIT | | |
| | | | | QEGISFYNDICGKVNSFMNLYCQKNKENKNLYKLQKLHKQILCIADTSY | | |
| | | | | EVPYKFESDEEVYQSVNGELDNISSKHIVERLRKIGDNYNGYNLDKIYI | | |
| | | | | VSKFYESVSQKTYRDWETINTALEIHYNNILPGNGKSKADKVKKAVKND | | |
| | | | | LQKSITEINELVSNYKLCSDDNIKAETYIHEISHILNNFEAQELKYNPE | | |
| | | | | IHLVESELKASELKNVLDVIMNAFHWCSVFMTEELVDKDNNEYAELEEI | | |
| | | | | YDEIYPVISLYNLVRNYVTQKPYSTKKIKLNEGIPTLADGWSKSKEYSN | | |
| | | | | NAIILMRDNLYYLGIFNAKNKPDKKIIEGNTSENKGDYKKMIYNLLPGP | | |
| | | | | NKMIPKVELSSKTGVETYKPSAYILEGYKQNKHIKSSKDFDITECHDLI | | |
| | | | | DYEKNCIAIHPEWKNEGFDFSDTSTYEDISGFYREVELQGYKIDWTYIS | | |
| | | | | EKDIDLLQEKGQLYLFQIYNKDFSKKSTGNDNLHTMYLKNLFSEENLKD | | |
| | | | | IVLKLNGEAEIFFRKSSIKNPIIHKKGSILVNRTYEAEEKDQFGNIQIV | | |
| | | | | RKNIPENIYQELYKYENDKSDKELSDEAAKLKNVVGHHEAATNIVKDYR | | |
| | | | | YTYDKYFLHMPITINFKANKTGFINDRILQYIAKEKDLHVIGIDRGERN | | |
| | | | | LIYVSVIDTCGNIVEQKSFNIVNGYDYQIKLKQQEGARQIARKEWKEIG | | |
| | | | | KIKEIKEGYLSLVIHEISKMVIKYNAIIAMEDLSYGEKKGRFKVERQVY | | |
| | | | | QKFETMLINKLNYLVEKDISITENGGLLKGYQL | | |

I claim:

1. A nucleic acid-guided nuclease editing system comprising:
an N-terminal nuclease domain/first transcription factor fusion construct present on a vector backbone;
a C-terminal nuclease domain/second transcription factor fusion construct present on a vector backbone, wherein the first transcription factor and the second transcription factor associate with one another in the presence of a ligand;
an editing cassette comprising a gRNA transcription sequence and a donor DNA transcription sequence present in an editing vector backbone; and
a binding site for the associated first and second transcription factors present on the editing vector backbone.

2. The nucleic acid-guided nuclease editing system of claim 1, wherein the N-terminal nuclease domain/first transcription factor fusion construct and the C-terminal nuclease domain/second transcription factor fusion construct are present on different vectors.

3. The nucleic acid-guided nuclease editing system of claim 1, wherein the N-terminal nuclease domain/first transcription factor fusion construct and the C-terminal nuclease domain/second transcription factor fusion construct are present on a first vector.

4. The nucleic acid-guided nuclease editing system of claim 3, wherein the N-terminal nuclease domain/first transcription factor fusion construct and the C-terminal nuclease domain/second transcription factor fusion construct are under the control of a single promoter.

5. The nucleic acid-guided nuclease editing system of claim 3, wherein the N-terminal nuclease domain/first transcription factor fusion construct and the C-terminal nuclease domain/second transcription factor fusion construct are under the control of separate promoters.

6. The nucleic acid-guided nuclease editing system of claim 1, wherein the first and second transcription factors are a same transcription factor that dimerize in the presence of the ligand.

7. The nucleic acid-guided nuclease editing system of claim 6, wherein the transcription factors are selected from AraR, XlrR, AP-1 (activator protein 1), C/EBP (CCAAT-enhancer binding protein, ATF/CREB activating transcription factor/cAMP response binding element, c-Myc, or NF-1 nuclear factor 1.

8. The nucleic acid-guided nuclease editing system of claim 1, wherein the N-terminal nuclease domain and the C-terminal nuclease domains are derived from MAD7 nuclease.

9. The nucleic acid-guided nuclease editing system of claim 8, wherein the MAD7 nuclease is split in a position as enumerated in Table 2.

10. The nucleic acid-guided nuclease editing system of claim 1, further comprising a coding sequence for the ligand.

11. The nucleic acid-guided nuclease editing system of claim 10, wherein the coding sequence for the ligand is under the control of an inducible promoter.

12. The nucleic acid-guided nuclease editing system of claim 10, wherein the coding sequence for the ligand is on the editing vector.

13. A nucleic acid-guided nuclease editing system comprising:
an N-terminal nuclease domain/first transcription factor fusion construct present on a vector backbone;
a C-terminal nuclease domain/second transcription factor fusion construct present on a vector backbone, wherein the first transcription factor and the second transcription factor associate with one another in the presence of a ligand;
a coding sequence for the ligand;
an editing cassette comprising a gRNA transcription sequence and a donor DNA transcription sequence present in an editing vector backbone; and
a binding site for the associated first and second transcription factors present on the editing vector backbone.

14. The nucleic acid-guided nuclease editing system of claim 13, wherein the N-terminal nuclease domain/first transcription factor fusion construct and the C-terminal nuclease domain/second transcription factor fusion construct are present on a first vector.

15. The nucleic acid-guided nuclease editing system of claim 14, wherein the N-terminal nuclease domain/first transcription factor fusion construct and the C-terminal nuclease domain/second transcription factor fusion construct are under the control of a single promoter.

16. The nucleic acid-guided nuclease editing system of claim 15, wherein the transcription factors are selected from AraR, XlrR, AP-1 (activator protein 1), C/EBP (CCAAT-enhancer binding protein, ATF/CREB activating transcription factor/cAMP response binding element, c-Myc, or NF-1 nuclear factor 1.

17. The nucleic acid-guided nuclease editing system of claim 13, wherein the N-terminal nuclease domain and the C-terminal nuclease domains are derived from MAD7 nuclease.

18. The nucleic acid-guided nuclease editing system of claim 17, wherein the MAD7 nuclease is split in a position as enumerated in Table 2.

19. The nucleic acid-guided nuclease editing system of claim 13, wherein the first and second transcription factors are a same transcription factor that dimerize in the presence of the ligand.

20. The nucleic acid-guided nuclease editing system of claim 13, wherein the first and second transcription factors are different transcription factors.


SEQUENCE LISTING (54 sequences)

SEQ ID NO: 1 - DNA, 20 bp, Artificial Sequence
  TRANSCRIPTION FACTOR FRAGMENT
taatattgtacgaacaattt

SEQ ID NO: 2 - DNA, 10 bp, Artificial Sequence
  TRANSCRIPTION FACTOR FRAGMENT
attgcgcaat

SEQ ID NO: 3 - DNA, 15 bp, Artificial Sequence
  TRANSCRIPTION FACTOR FRAGMENT
  misc_feature (6)..(10): n = ANY NUCLEOTIDE
ttggcnnnnngccaa

SEQ ID NO: 4 - protein, 629 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFD

SEQ ID NO: 5 - protein, 634 aa, Eubacterium rectale
ITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 6 - protein, 631 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDIT

SEQ ID NO: 7 - protein, 632 aa, Eubacterium rectale
FCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 8 - protein, 633 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFC

SEQ ID NO: 9 - protein, 630 aa, Eubacterium rectale
HDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 10 - protein, 635 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHD

SEQ ID NO: 11 - protein, 626 aa, Eubacterium rectale
DYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 12 - protein, 639 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDY

SEQ ID NO: 13 - protein, 624 aa, Eubacterium rectale
FKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 14 - protein, 641 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFK

SEQ ID NO: 15 - protein, 622 aa, Eubacterium rectale
NCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 16 - protein, 643 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNC

SEQ ID NO: 17 - protein, 620 aa, Eubacterium rectale
IAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 18 - protein, 645 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIA

SEQ ID NO: 19 - protein, 620 aa, Eubacterium rectale; identical to SEQ ID NO: 17

SEQ ID NO: 20 - protein, 645 aa, Eubacterium rectale; identical to SEQ ID NO: 18

SEQ ID NO: 21 - protein, 618 aa, Eubacterium rectale
IHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 22 - protein, 647 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIH

SEQ ID NO: 23 - protein, 616 aa, Eubacterium rectale
PEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 24 - protein, 847 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITI

SEQ ID NO: 25 - protein, 416 aa, Eubacterium rectale
NFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 26 - protein, 849 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINF

SEQ ID NO: 27 - protein, 414 aa, Eubacterium rectale
KANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 28 - protein, 851 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKA

SEQ ID NO: 29 - protein, 412 aa, Eubacterium rectale
NKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 30 - protein, 853 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANK

SEQ ID NO: 31 - protein, 410 aa, Eubacterium rectale
TGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 32 - protein, 991 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKL

SEQ ID NO: 33 - protein, 272 aa, Eubacterium rectale
NYLVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 34 - protein, 993 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNY

SEQ ID NO: 35 - protein, 270 aa, Eubacterium rectale
LVFKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 36 - protein, 995 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLV

SEQ ID NO: 37 - protein, 268 aa, Eubacterium rectale
FKDISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 38 - protein, 997 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFK

SEQ ID NO: 39 - protein, 266 aa, Eubacterium rectale
DISITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 40 - protein, 999 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDI

SEQ ID NO: 41 - protein, 264 aa, Eubacterium rectale
SITENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 42 - protein, 1001 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISI

SEQ ID NO: 43 - protein, 262 aa, Eubacterium rectale
TENGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 44 - protein, 1003 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITE

SEQ ID NO: 45 - protein, 260 aa, Eubacterium rectale
NGGLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 46 - protein, 1005 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENG

SEQ ID NO: 47 - protein, 258 aa, Eubacterium rectale
GLLKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 48 - protein, 1007 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGL

SEQ ID NO: 49 - protein, 256 aa, Eubacterium rectale
LKGYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 50 - protein, 1009 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLK

SEQ ID NO: 51 - protein, 254 aa, Eubacterium rectale
GYQLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 52 - protein, 1011 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGY

SEQ ID NO: 53 - protein, 252 aa, Eubacterium rectale
QLTYIPDKLKNVGHQCGCIFYVPAAYTSKIDPTTGFVNIFKFKDLTVDAKREFIKKFDSIRYDSEKNLFCFTFDYNNFITQNTVMSKSSWSVYTYGVRIKRRFVNGRFSNESDTIDITKDMEKTLEMTDINWRDGHDLRQDIIDYEIVQHIFEIFRLTVQMRNSLSELEDRDYDRLISPVLNENNIFYDSAKAGDALPKDADANGAYCIALKGLYEIKQITENWKEDGKFSRDKLKISNKDWFDFIQNKRYL

SEQ ID NO: 54 - protein, 1013 aa, Eubacterium rectale
MNNGTNNFQNFIGISSLQKTLRNALIPTETTQQFIVKNGIIKEDELRGENRQILKDIMDDYYRGFISETLSSIDDIDWTSLFEKMEIQLKNGDNKDTLIKEQTEYRKAIHKKFANDDRFKNMFSAKLISDILPEFVIHNNNYSASEKEEKTQVIKLFSRFATSFKDYFKNRANCFSADDISSSSCHRIVNDNAEIFFSNALVYRRIVKSLSNDDINKISGDMKDSLKEMSLEEIYSYEKYGEFITQEGISFYNDICGKVNSFMNLYCQKNKENKNLYKLQKLHKQILCIADTSYEVPYKFESDEEVYQSVNGFLDNISSKHIVERLRKIGDNYNGYNLDKIYIVSKFYESVSQKTYRDWETINTALEIHYNNILPGNGKSKADKVKKAVKNDLQKSITEINELVSNYKLCSDDNIKAETYIHEISHILNNFEAQELKYNPEIHLVESELKASELKNVLDVIMNAFHWCSVFMTEELVDKDNNFYAELEEIYDEIYPVISLYNLVRNYVTQKPYSTKKIKLNFGIPTLADGWSKSKEYSNNAIILMRDNLYYLGIFNAKNKPDKKIIEGNTSENKGDYKKMIYNLLPGPNKMIPKVFLSSKTGVETYKPSAYILEGYKQNKHIKSSKDFDITFCHDLIDYFKNCIAIHPEWKNFGFDFSDTSTYEDISGFYREVELQGYKIDWTYISEKDIDLLQEKGQLYLFQIYNKDFSKKSTGNDNLHTMYLKNLFSEENLKDIVLKLNGEAEIFFRKSSIKNPIIHKKGSILVNRTYEAEEKDQFGNIQIVRKNIPENIYQELYKYFNDKSDKELSDEAAKLKNVVGHHEAATNIVKDYRYTYDKYFLHMPITINFKANKTGFINDRILQYIAKEKDLHVIGIDRGERNLIYVSVIDTCGNIVEQKSFNIVNGYDYQIKLKQQEGARQIARKEWKEIGKIKEIKEGYLSLVIHEISKMVIKYNAIIAMEDLSYGFKKGRFKVERQVYQKFETMLINKLNYLVFKDISITENGGLLKGYQL